(19) United States
(12) Patent Application Publication
(10) Pub. No.: US 2010/0028302 A1
(43) Pub. Date: Feb. 4, 2010

(54) Use of IGFBP-2 in Senescence Diseases and for the Maintenance of Organ Functions

(75) Inventors: Andreas Hõflich, Gulzow/Wilhelmienhof (DE); Eckhard Wolf, Vierkirchen (DE); Ingrid Renner-Müller, Vierkirchen (DE); Petra Renner, Munchen (DE); Daniela Diehl, Freising (DE); Max Bielohuby, Muichen (DE); Esther Hessel, Basel (CH)

Correspondence Address: Pearl Cohen Zedek Latzer, LLP, 1500 Broadway, 12th Floor, New York, NY, 10036, US

(73) Assignee: LUDWIG-MAXIMILIANS- UNIVERSITÄT MÜNCHEN, Munich (DE)

(21) Appl. No.: 11/663,784

(22) PCT Filed: Sep. 26, 2005
(86) PCT No.: PCT/EP05/10389

§ 371 Date: Jul. 29, 2009

(30) Foreign Application Priority Data
Sep. 27, 2004 (EP) 04022973.4

(51) Int. Cl. A61K 35/12; A61K 38/16; A61K 31/7088

(52) U.S. Cl. 424/93.2; 514/12; 514/44 R

(57) ABSTRACT

The present invention relates to the use of an IGFBP-2 (insulin-like growth factor binding protein-2) molecule for the production of a pharmaceutical composition for the regulation of senescence processes in cells, tissues and/or organs for the maintenance and control of tissue and/or organ functions and/or for the treatment or alleviation of senescence symptoms or early senescence, wherein the IGFBP-2 molecule is selected from the group of an IGFBP-2 polypeptide or of a functional fragment thereof and of a nucleic acid encoding an IGFBP-2 polypeptide or a functional fragment or derivative thereof. Moreover, corresponding methods of treatment are provided. The use of IGFBP-2 in the preparation of a pharmaceutical composition in the medical intervention of cachexia is described.

The present invention relates to the use of an IGFBP-2 (insulin-like growth factor binding protein-2) molecule for the production of a pharmaceutical composition for the regulation of senescence processes in cells, tissues and/or organs for the maintenance of tissue and/or organ functions and/or for the treatment or alleviation of senescence symptoms or early senescence, wherein the IGFBP-2 molecule is selected from the group of an IGFBP-2 polypeptide or of a functional fragment thereof and of a nucleic acid encoding an IGFBP-2 polypeptide or a functional fragment or derivative thereof. Also provided is the use of IGFBP-2 in the medical intervention of proliferative and/or cancerous disease and the use of IGFBP-2 in the augmentation of body fat (and/or body mass) in patients, in particular patients with cachexic phenotype (cachexia). Moreover, corresponding methods of treatment are provided.

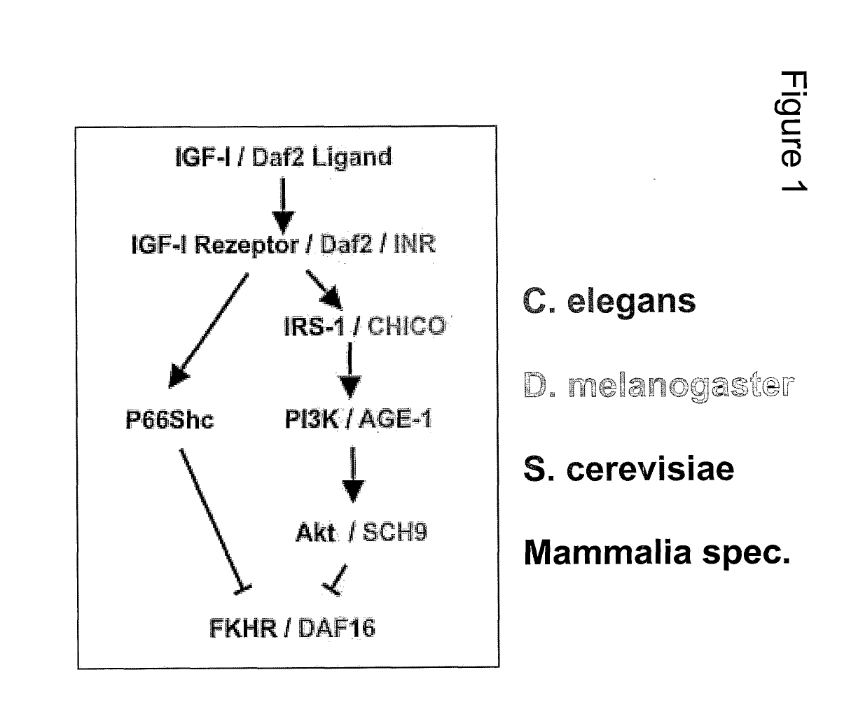

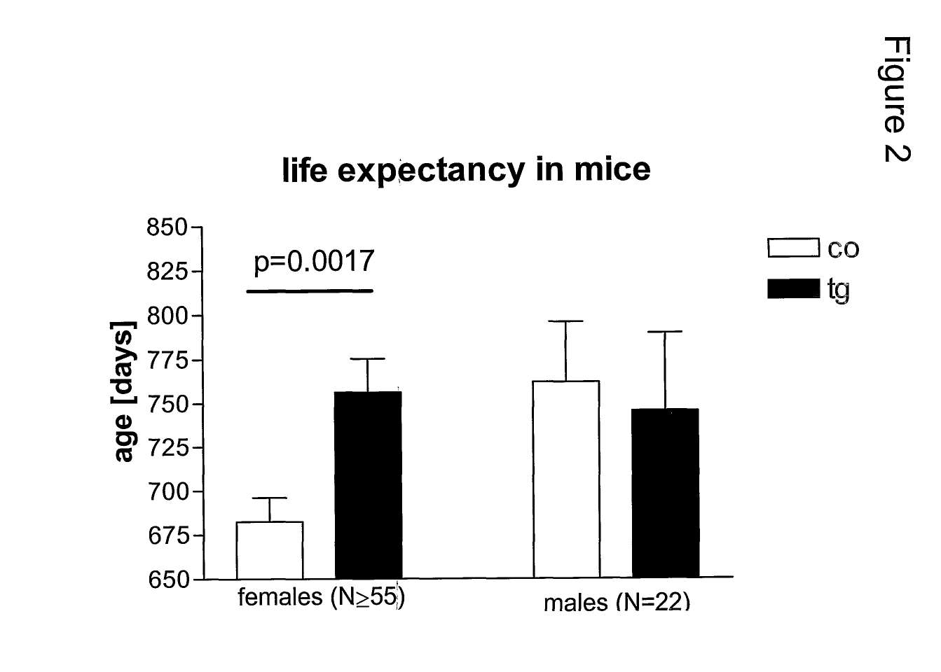

Senescence processes are extremely complex and until today no uniform definition of the term “senescence” exists. The reason is surely that the mechanism of senescence and the responsible genes have until now only been detected very insufficiently (Hamat & Tremblay 2003). To date almost exclusively the PI3-Kinase pathway has received intense research with respect to its role for controlling life expectancy. Therefore, among the few genes which have been identified as “senescence genes” there are numerous representatives of the IGF system or IGF-dependent signal cascades. This aspect of the IGF-mediated biological effects is highly conserved from an evolutionary point of view. In C. elegans, D. melanogaster, S. cerevisiae but also in the mouse individual orthologous proteins have been identified which are involved in the control of life expectancy ((Barbieri 2003); FIG. 1). In C. elegans specific effects on life expectancy were detected for DAF-2 (orthologous to the insulin-/IGF-I receptor), AGE-1 (orthologous to a subunit of pI3K) and DAF-16 (orthologous to the FOXO gene family of the Forkhead transcription factors). In D. melanogaster, the orthologous genes for the regulation of life expectancy are INR (insulin receptor) and CHICO (IRS-1). In yeasts, it has been shown that SCH9 controls life expectancy. SCH9 has similarities to AKT1/AKT2 which, in turn, are regulated by IGF-I.

Only recently, an active role of IGF-IR in senescence was detected in mice. As the absolute lack of IGF-IR is lethal, mice were examined in which only one single allele of IGF-IR was deleted. These mice, which were characterised by a reduced IGF-IR concentration lived considerably longer than their control brothers and sisters (Holzenberger 2003). Thus, it can be assumed that certain IGF-I-dependent signal cascades play an important role in the control of life expectancy.

Additionally, the heterozygous IGF-IR knockout mice also had an increased resistance to oxidative stress. Presumably, p66Shc, a component of intracellular signal cascades plays an important role in this connection. Mice, whose p66Shc genes were inactivated, exhibited an increased life expectancy and an increased resistance to oxidative stress (Napoli 2003). As p66Shc can, via its PTB domain, also bind to other tyrosine kinase receptors (e.g. EGF receptor) apart from IGF-IR, this adaptor protein possibly plays a key role in the intracellular processing of extracellular signals. It is interesting to note that the activation of p66Shc does not result in an activation of the MAPK pathway, as would be the case after binding of p46Shc and p52Shc, but eventually leads to an inactivation of FKHR transcription factors (Purdom & Chen 2003). The influence of p66She on life expectancy and oxidative stress is mediated by FKHR, which in an active (unphosphorylated) state reduces the lifespan of cells and the oxidative stress.

However, research covering control of ageing and the knowledge on the respective genes is at an extremely preliminary level. Novel high throughput technologies permanently identify new candidate genes for the control of lifespan. As an example one study published 23 novel candidate genes for the control of life expectancy and most of them had nothing to do with the IGF-system. These data are clearly contradictory to the former assumption of a dominant role of the IGF-system during the control of ageing (Hansen 2005). One of the main conclusions drawn from their results was that particularly integrin-signalling might hold a central and evolutionary conserved position for the control of life expectancy.

It is furthermore known that life span also depends on dietary control (Weindruch 1986). The only thing known on the mechanism behind dietary control of life span control is that the insulin/IGF-system is not involved (Lakowski and Hekimi 1985; Houthooft 2003). Very recently, a novel mechanism has been found for the control of forkhead transcription factors and thus life-span (Essers 2005). It was found that β-catenin, via binding to FOXO, has an effect on the activity of redox-relevant enzymes (SOD) and thereby affects life span. Thus β-catenin, which is known to stimulate cell proliferation through the LEF/TCF-pathway (Reya and Clevers 2005), now also is capable to block progression of the cell cycle and to modulated life expectancy. Classically, β-catenin is dependent on Wnt-signalling (Logan and Nusse 2004). Degradation of β-catenin is initiated by glycogen synthase kinase 3β (GSK-3β) dependent phosphorylation (Ali 2001). Thus, GSK-3β has a central function for the control of the Wnt-pathway. The activity of the GSK-3β underlies complex control through G-proteins (PKA), tyrosine kinases (PKB), the calcineurin/NFAT pathway (PKCs) as well through integrins (Dorn and Force 2005).

Apparently, the Wnt-signalling pathway is important for the interactions between cells and environment (cell/cell- or cell/extracellular matrix contact; (Schambony 2004)) and during ontogeny of the individuum (Han 2005). Particularly proteoglycans and integrins are of critical importance to the Wnt-signalling pathway (Alexander 2000; Novak 1998; Song 2005). Very recent publications also demonstrate functional relevance of proteoglycans for the malignant potential of the Wnt-signalling pathway (Capurro 2005).

Coshigano (2003, Endocrinology, 144, 3799-3810) describes a mutation study in the insulin-dependent signal system. In the study, homozygous, transgenic knockout mice were produced which exhibited no growth hormone receptor (GHR). These mice exhibited low growth. In a sober state, they exhibited a low insulin level and also a lower glucose level. These mice exhibited reduced IGFBP-1 and -4 levels, however, the IGFBP-2 values were increased. Independent of their sex, the homozygous mice exhibited a higher life expectancy. A correlation between life expectancy and IGFBP-2 serum values was neither shown nor detected.

The insulin-like growth factor (IGF) system consists of the peptide hormones IGF-I and IGF-II, six highly affinitive IGF-binding proteins (IGFBP-1 to -6) and two IGF receptors (IGF-I receptor: IGF-IR and IGF-II/mannose-6-phosphate receptor: IGF-IIR; FIG. 1). The IGF-IR consists of two extracellular and two membrane-bound subunits. It is said to play a fundamental role in the IGF-dependent induction of mitogenic and antiapoptotic signal cascades (De Meyts & Whittaker 2002; LeRoith 1996). The IGF-IIR is a multifunctional receptor, which amongst others also participates in the degradation of IGF-II and is assumed to have a connection to the induction of cell differentiation (Ghosh 2003). The IGFBPs are expressed tissue-specifically and are supposed to be important modulators of the local effects of the IGFs (Firth & Baxter 2002). Moreover, the IGFBPs also induce IGF-independent effects.

The IGFBPs can bind to cell surfaces in different ways. In this connection, it is assumed that certain heparin-binding domains of IGFBP-2, -3, -5 and -6 or the RGD domains of IGFBP-1 and -2 participate (Parker 1998; Fowlkes 1997; Brewer 1988). IGFBP-1 interacts, via its RGD domain, with α5β1 integrins and can, thus, influence the cell proliferation and cell adhesion (Irving & Lala 1995; Irwin & Giudice 1998). Above all, IGFBP-2 is often markedly increased in tumour cells and an active participation in malignant growth was shown in different cell systems. Interestingly, IGFBP-2 induces the expression of genes the products of which promote the invasion of tumour cells (Wang 2003). One of these genes codes for a matrix metalloprotease (MMP-2) whose proteolytic activity is necessary for the degradation of the basal membrane. Recently, a new IGFBP-2 binding protein was identified and was named invasion-inhibitory protein IIp45 according to its invasion-inhibiting property (Song 2003). Possibly, this protein prevents that IGFBP-2 attaches to integrins and interacts with the cellular signalling. IGFBP-2, however, also binds to proteoglycans (Russo 1997 and 2005) and α5β1 integrins (Pereira 2004). Both receptors are relevant for the adhesive properties of the corresponding cell, however, the consequence of this interaction in viva remains unknown. IGFBP-2 can translocate under oxidative stress and it is assumed that IGFBP-2 exerts stress-adaptive effects in the nucleus (Besnard 2001) It was possible to detect an effect of IGFBP-2 on the expression of redox-relevant enzymes in vitro (Hoeflich 2003). It was speculated, that the effects of IGFBP-2 on redox-relevant enzymes in Y1 tumour cells are causative for the increased malignant potential of IGFBP-2 overexpressing tumour cells.

Interestingly, the concentration of serum IGFBP-2 increases as humans age (van den Beld 2003). Moreover, high serum levels were correlated with a poorer general state of health, while lower serum concentrations of IGFBP-2 induced a better constitution. The results of this study, thus, led to the conclusion that IGFBP-2 itself is actively responsible for the poor general state of health.

Insulin-like growth factors (IGF-I and IGF-II) are effective mitogens in numerous normal and malignant cells. Growing evidence leads to the assumption that IGFs play an important role in the pathophysiology of prostate diseases and breast tumours (Boudon (1996), J. Clin. Endocrin. Metap. 81: 612-617; Angeloz-Nicoud (1995), Endocrinology 136: 5485-5492; Nickerson (1998), Endocrinology 139: 807-810; Figueroa (1998), J. Urol. 159: 1379-1383). In IGF-responsive cells IGFBP-2 had growth inhibitory potential, whereas in tumour cells IGFBP-2 is believed to enhance malignant growth independent or dependent of the IGFs (Hoeflich Cancer Res 2001).

As indicated above, IGFBPs (insulin-like growth factor binding proteins 1-6) are proteins with a length of 216 to 300 (optionally 305 and also more) amino acid residues, wherein the mature IGFBP-5 consists of e.g. 252 amino acid residues (Wetterau 1 (1999), Mol. Gen. Metap. 68: 161-181). Inherently, all IGFBPs have a similar organisation of their protein domains. The strongest conservation can be found in the N-(amino acid residues 1 to approx. 100) and C-(starting at amino acid residue 170) terminal cystein-rich region. 12 preserved cystein residues can be found in the N-terminal domain and 6 in the C-terminal protein domain. The central, poorly preserved part (L-protein domain) contains most cleavage sites of specific proteases (Chemausek (1995), J. Biol. Chem., 270, 11377-11382). Until today, a lot of different fragments of IGFBPs have been described and biochemically characterised (Mazerburg (1999), Endocrinology, 140, 4175-4184; Mark (2005), Biochemistry, 44, 3644-3652). Mutagenesis studies lead to the assumption that the high-affinity IGF binding site is localised in the N-terminal domain (Wetterau (1999), loc.cit.; Cheranausek (1995), loc.cit.) and that at least IGFBP-3 and IGFBP-2 have 2 binding sites, one in the N— and another one in the C-terminal protein domain (Wetterau (1999), loc. cit.). Recently, IGFBP-related proteins (IGFBP-rPs) were described which bind with a lower affinity than IGFBPs (Hwa “The ETF-binding protein superfamily”, (1999), Humana Press Totowa, 315-327). IGFBPs and IGFBP-rPs both have the highly preserved and cystein-rich N-terminal protein domain which seems to be necessary for a numerous biological processes, including the binding to the IGFs and the high-affinity binding to insulin (Hwa (1999), loc. cit.). The N-terminal fragments of IGFBP-3 which are produced e.g. by enzymatic cleavage, also bind insulin and are, thus, probably physiologically relevant for the insulin metabolism. After the N-terminal domain, the sequence similarity between the IGFBPs and the IGFBP-rPs ends.

Due to the linking of the insulin-like growth factor (IGF) with neoplasia, it is apparent that the inhibition of the IGF signal pathway in tumours might possibly be a successful strategy in cancer therapy. Such a modulation was proposed by an exogenous administration of recombinant inhibitory IGFBPs and physiologically-effective fragments thereof. Additionally, it was proposed to influence the IGFBP production, inhibition or degradation in tumour cells by active agents such as Tamoxifen and ICE182 780 (Khandwalla (2000), Endocr. Ref., 21, 215-244).

In vitro, IGFBPs exhibit a significant inhibition of the proliferation of tumour cells, whereas only very high doses are in vivo effective to inhibit tumour growth (Van den Berg (1997), Eur. J. Cancer, 33, 1108-1113). To this avail, Van den Berg coupled IGFBP-1 to polyethylene glycol, via a covalent binding, which led to an increase of the half-life in serum. Nevertheless, the inhibitory effect of the polyethylene glycosylated IGFBP-1 is still not sufficient for a therapeutic application in humans as only a partial response could be detected, even when polyethylene glycosylated IGFBP-1 was administered to mice in doses of 1 mg/dose. This corresponds to a dose of 50 mg/kg per day, which, according to established methods, cannot be administered to humans and could not be produced economically.

Increased concentrations of IGFBP-2 were detected in human tumour tissue including adrenocortical carcinoma. In order to elucidate the functional effects of an IGFBP-2 overexpression, the cDNA of murine IGFBP-2 was stably transfected into murine adrenocortical tumour cells (Y-1). A long-time overexpression of IGFBP-2 was connected with significant morphological changes, an increased cell proliferation and an increased efficiency in cloning compared to the mock-transfected control cells. The increased proliferation of IGFBP-2-secreting clones was independent of exogenous insulin-like growth factors (IGFs). These results lead to the assumption that an increased IGFBP-2-level possibly contributes to the highly malignant phenotype of adrenocortical cancer by a mechanism, which is IGF-independent and unknown until today; cf. Hoeflich (2000), Cancer Res., 60, 834-838. It was then stated that IGFBP-2 is protective in normal cells and malignant in tumour cells (Hoeflich (2001) loc. cit.; Moore (2003), Int J. Cancer 105, 14-19).

IGFBP-2 mRNA is already expressed in pre-implanted embryos (Prelle (2001), Endocrinology, 142, 1309-1316) and the expression continues on a high level in many tissues during embryogenesis and the fetal development (Schuller (1993), Endocrinology, 132, 2544-2550; von Kleffens (1998), Mol. Ser. Endocrinol. 140, 129-135). Post-natal, IGFBP-2 is the second most common IGFBP in the circulation and is present in different other biological fluids and tissues in a lot of species of vertebrates (Blum (1993), Growth Regul., 3, 100-104; Hwa (1999), Endocr. Rev., 20, 761-787).

As mentioned above, the IGFBP-2 serum concentration is increased in a lot of acute or chronic non-physiologic situations such as shock, hunger, hypoxemia or after traumata, which leads to the assumption that the IGFBP-2 expression has a complex regulation. Moreover, increased IGFBP-2 concentrations in the serum are associated with a reduced growth in body height in mice, which were selected for a low body weight (Hoeflich (1998), Growth Horm. IGF Res., 8, 113-123).

The targeted inactivation of the IGFBP-2 gene in mice only led to subtle consequences for the phenotype, possibly by the functional compensation by other IGFBPs for which, in this model, an up-regulation was detected (Toth (1993), Growth Regul., 3, 5-8; Pintar (1995), Prog. Growth Factor Res., 6, 437-445; Wood (2000), Mol. Endocrinol., 14, 1472-1482). Contrary to transgenic mice which exhibited an overexpression of the IGFBP-2 gene due to a CMV promoter, they showed a significantly reduced body weight, which leads to the assumption that IGFBP-2 is a negative regulator of normal somatic growth, probably by the excretion of IGFs by their receptors (Hoeflich (1999), Endocrinology, 140, 5488-5496; Schneider (2000), FASEB J., 14, 629-640; Wolf (2000), Pediatr. Nephrol., 14, 572-578). The inhibitory effect of IGFBP-2 was even stressed when the CMV-IGFBP-2 transgenic mice were interbred into a transgenic mouse model with increased GH and IGF-I levels, whereby the concept that IGFBP-2 is an IGF-dependent growth inhibitor in vivo was supported (Hoeflich (2001), Endocrinology, 142, 1889-1898).

In WO 03/062421, bispecific antisense oligonucleotides are described which inhibit IGFBP-2. These bispecific antisense oligonucleotides are in particular to be used in the treatment of endocrine-regulated tumours (such as e.g. breast, prostate, ovarian and colon cancer).

WO 02/098914 describes specific mutants of IGF binding proteins and in particular describes methods for the production of corresponding antagonists. In particular, crystalline structures for X-ray diffractions are provided which provide a complex of insulin-like growth factor 1 or 2 (IGF-I or IGF-II) and a polypeptide which particularly comprises amino acids 55 to 107 of IGFBP-2.

WO 2004/033481 provides peptides or small molecules derived from IGFBP. The molecules described therein are particularly derived from IGFBP-3 and are to be used in the treatment of various diseases such as cancer, autoimmune diseases, cardiovascular indications, arthritis, asthma, allergies, indications of the reproduction tract, in proliferative diseases of the retina, in bone diseases, in inflammations, in inflammatory gastroenteropathies and in fibrotic diseases.

In US 2003/0087806 a pharmaceutical composition consisting of a complex insulin-like growth factor (IGF) and insulin-like growth factor binding protein (IGFBP) is described. This formulation is in particular stabilised without additional osmolytic salts. The administration of IGF in combination with the complex builder IGFBP is proposed, in particular, in order to avoid or suppress side effects of the IGF administration in the medicinal context (e.g. in the treatment of diabetes or amyotrophic lateral sclerosis) described.

In WO 00/96454 the suppression of endogenous IGFBP-2 for the inhibition of cancer diseases is proposed. In particular, modulators are provided which are to be used for the treatment of cancer in any tissues, in particular in prostate tissue. The modulators are in particular inhibiting IGFBP-2 molecules.

In US 2004072776 antisense oligonucleotides are provided which inhibit IGFBP-2 and are in particular to be used in the prostate tumour therapy and other endocrine tumour therapies.

In U.S. Pat. No. 6,025,332 methods of treatment are proposed for the treatment of physiological-psychological diseases, metabolic diseases, chronic stress diseases, sleep disorders and medicinal conditions, which are linked to sexual senescence conditions or senescence. These methods of treatment in particular comprise the administration of IGF or mutant IGF forms, which are to be administered either alone or in combination with IGFBP-3.

Similarly, in U.S. Pat. No. 5,093,317, U.S. Pat. No. 5,420,11, U.S. Pat. No. 5,068,224, WO 93/02695, WO 93/08826 and WO 95/13823, the use of IGF or IGF/IGFBP-3 complexes for the treatment of diseases of the nervous system are described.

The technical problem underlying the present invention is the provision of methods, which can slow down the senescence processes in biological systems and in particular in mammal and/or which lead to cells, tissues and organs remaining longer in a positive physiological condition in vivo.

The solution to this problem is provided by the present invention and is in particular characterised in the claims and in the embodiments.

The present invention relates to the use of an IGFBP-2 (insulin-like growth factor binding protein-2) molecule for the production of a pharmaceutical composition for the regulation of senescence processes in cells, tissues and/or organs for the maintenance of tissue and/or organ functions and/or for the treatment or alleviation of senescence symptoms or early senescence,

wherein the IGFBP-2 molecule is selected from the group
of an IGFBP-2 polypeptide or of a functional fragment thereof and
of a nucleic acid encoding an IGFBP-2 polypeptide or a functional fragment or derivative thereof.

The appended data and the present invention show that IGFBP-2 is a novel anti-ageing agent and function in the maintenance of a non-pathological tissue and/or organ function. Accordingly, it is proposed in context of this invention that IGFBP-2 be used in the medical intervention of senescence, in particular early senescence as well as in the prevention, treatment and/or alleviation of proliferative disorders, like cancer and in particular of colon cancer and/or the treatment of cachexia.

As proved in the experimental part and hereinafter, it was surprisingly found that IGFBP-2 is a molecule which can be used for slowing down senescence processes and for the prevention of a tumour incidence, particularly in the liver. This is in clear contrast to the opinion previously published, namely that IGFBP-2 leads to poor physiological conditions, in particular in old age. Furthermore it is also illustrated that IGFBP-2 is capable of positively influencing the maintenance of tissue and/or organ function, for example the maintenance of a non-tumorous phenotype of said tissue and/or organ. As documented herein, IGFBP-2 is in this respect protective and prevents from proliferative disorders. This, again, is in clear contrast to previously published data, wherein IGFBP-2 was considered a causative agent for cancer and/or proliferative disorders. Several studies led to the conclusion that IGFBP-2 blocks proliferation of non-malignant cells and has the potential to stimulate growth of tumour cells (reviewed in Hoeflich (2001) loc. cit.; Moore (2003), loc. cit.). Consequently, in tumours blockade of IGFBP-2 expression was suggested in order to stop malignant growth. This common view in the scientific community is documented by various publications and patent applications which target IGFBP-2 and try to inhibit the expression or function of this protein. In light of the tumour protective properties of IGFBP-2 documented herein, the results provided herein direct to an opposite role of IGFBP-2 in tumour growth in vivo.

This is a surprising finding since IGFBP-2 was believed to represent a bifunctional protein: in an IGF-dependent mechanism it has been shown to exert negative growth effects, while in a malignant context (e.g. in tumor cells) malignant potential was attributed to IGFBP-2. As documented in the appended examples, a contrary protective effects both in highly senescent mice and during chemically induced carcinogenesis could be demonstrated. This shows that IGFBP-2 is a robust anticancer agent which can in fact be used to prevent cancer, surprisingly even if a cell is prone to cancer. Data presented in the appended examples suggest that treatment of tumours by using IGFBP-2 antisense molecules (as proposed, inter alia, in WO 03/062421) may be contra-indicated. Against the broad understanding it could surprisingly be shown that IGFBP-2 exerts protective effects against tumour growth in vivo in different approaches (senescence-associated tumour growth and tumour growth in cells prone to cancer due to e.g. chemical carcinogenesis).

As used herein, the term “IGFBP-2” means an insulin-like growth factor binding protein 2. As mentioned above, IGFBP-2 is a member of the insulin-like growth factor binding protein family. The IGFBP-2 used herein can be derived from any species, preferably from mammals. Human IGFBP-2 is particularly preferred. The term “IGFBP-2” comprises naturally occurring sequences and variants, in particular naturally occurring allelic variants. Human IGFBP-2 is e.g. accessible in pertinent data banks, e.g. under “Swiss Prot Accession Number P18065). In preferred embodiments, IGFBP-2 is the human IGFBP-2 as defined in SEQ ID NO:1 and 2 by the encoding nucleic acid or by the corresponding amino acid sequence. Preferably, IGFBP-2 molecules which are at least 70% homologous to the sequence described in SEQ ID NO:2, can be used. Particularly preferred are sequences which are at least 80%, more preferred at least 85%, more preferred at least 90%, more preferred at least 95% and particularly preferred at least 97% identical to the amino acid sequence shown in SEQ ID NO:2. Particular variants of the IGFBP-2 molecules which can be used according to the invention, also comprise variants, in particular recombinant variants. These recombinant variants can in particular be produced to achieve an improved degradation resistance and/or to specifically manipulate the interaction with particularly integrins and/or proteoglycans. The IGFBP-2 molecules which are described herein and which are to be used can comprise native, wild-type and mutated IGFBP-2 molecules and can be isolated from natural sources or can be produced by methods which are well-known to the person skilled in the field of molecular biology. In particular, expression vectors can be used which can express the IGFBP-2 molecules. In particular, such expression vectors comprise suitable transcriptional and/or translatory control signals. The corresponding methods comprise both in vitro DNA recombination methods and other synthetic methods. Corresponding methods can be taken from e.g. Maniates (1989), Molecular Cloning: “A Laboratory Manual”; Cold Spring Harbour Laboratories. According to this invention, in particular molecules can be used whose amino acid sequence comprises a sequence which corresponds to the amino acids 215 to 316 (C-terminal fragment according to Swiss Prot Accession Number P18065) of SEQ ID NO:2 or which comprises these amino acids. The homology region of individual species of this C-terminal fragment is very high and, e.g. between humans and mice amounts to 97%. Accordingly, IGFBP-2 molecules, which are to be used according to the invention, in particular also comprise molecules which comprise a C-terminal part which is at least 90%, preferably at least 95% identical with the C-terminal part as in the amino acids 215 to 316 of the human IGFBP-2, as is known from Swiss Prot Accession Number PI8065 or as shown in SEQ ID NO:2. On the level of the amino acids, human IGFBP-2 is on its entire length 88% homologous to murine IGFBP-2. As mentioned above, the invention comprises the use of IGFBP-2 molecules which, in their amino acid sequence, are at least 80% homologous to the amino acid sequence as shown in SEQ ID NO:2.

According to the invention, the term “IGFBP-2” can also comprise further substances which can have the effect of IGFBP-2. These substances can, e.g. be low-molecular substances.

Due to the experimental teaching, the skilled person can test IGFBP-2 molecules (i.e. e.g. peptides, proteins, variants, derivatives) as described herein and also such low-molecular substances for their respective effectiveness. E.g. in cellular or somatic systems, the enzymatic activity of redox-relevant enzymes and/or the activity of FKHR can be determined in order to test whether the corresponding substances are to be used as IGFBP-2 molecules.

The term “nucleic acid” comprises polynucleotides which are in particular present in the form of a DNA, RNA, cDNA. The term also comprises synthetically produced polynucleotides and recombinant nucleic acid molecules. Corresponding further, but not concluding explanations are provided herein.

The term “pharmaceutical composition” as used herein comprises formulations of the IGFBP-2 molecules described herein which can be administered in particular for the systemic and for the topical administration to patients or to persons in need of a treatment described herein. The pharmaceutical formulations/pharmaceutical compositions comprise a therapeutically effective amount of the IGFBP-2 molecule, normally with a pharmaceutically active carrier or excipient.

The formulation should be suitable for the kind of administration and lies within the ability of the field. The invention moreover relates to pharmaceutical packages and kits comprising one or more containers, filled with one or several of the components of the IGFBP-2 compositions mentioned above.

The IGFBP-2 molecules can be administered alone or in combination with other compounds such as therapeutic compounds.

Preferred forms of systemic administration of IGFBP-2 pharmaceutical compositions comprise an injection, typically an intravenous injection. Other ways of injections such as subcutaneous, intramuscular or intraperitoneal can be used. Alternative possibilities for a systemic administration include intramucosal and transdermal administration by using permeation means such as bile acids or fusidinic acids or other detergents. Moreover, oral administration can also be possible and desirable. The desired dosage range depends on the choice of the IGFBP-2 to be administered, the route of administration, the nature of the formulation, the nature of the condition of the person and the evaluation by the physician in charge. Suitable dosages are in the range of 0.1 to 500 μg/kg body weight. Other proposals for the administration are indicated below. In view of the different effectiveness of the different routes of administration, however, it is expected that there are considerable variations with respect to the dosage required. For example, it would be expected that in case of oral administration, higher doses are required than in case of an administration by intravenous injection. Variations in these dosage amounts can be adapted using empiric standard routines for optimisation, as is well-known in the field. IGFBP-2 used in treatments can also be produced endogenously in the person, in treatment modalities which are often referred to as “gene therapy” as described above. Thus, for example, cells of a person can be modified with a polynucleotide, such as DNA or RNA in order to encode an IGFBP-2 ex vivo, for example by using a retroviral plasmid vector. The cells or even the vector itself are then introduced into the person.

The pharmaceutical composition according to the invention which is to be used can moreover comprise a pharmaceutically acceptable carrier. Examples of pharmaceutically acceptable carriers are well-known from the field and comprise phosphate-buffered saline solutions, water, emulsions such as oil/water emulsions, different kinds of dampening agents, sterile solutions, etc. Compounds comprising such carriers can be formulated with well-known standard methods. These pharmaceutical compositions can be administered to the patient in a suitable dose. The administration of the suitable compounds can be carried out by different routes, e.g. by intravenous, intraperitoneal, subcutaneous, intramuscular, topical or intradermal administration. The dosage scheme is determined by the physician who is present and by clinical factors. As is well-known in medicine, the dose for each patient depends on numerous factors, including the height or the weight of the patient, the body surface, the age, the particular compound to be administered, the sex, the period and the route of administration, the general state of health and other pharmaceutical compositions which are administered at that time. In general, the scheme as regular administration of pharmaceutical compositions should be in the range of 1 μg to 10 mg units per day. If the therapeutic scheme contains a continual infusion, it should also be in the range of in each case 1 μg to 10 mg units per kilogramme body weight per minute. However, a more preferred dosage for the continued infusion could be in the range of 0.01 μg to 10 mg units per kilogram body weight per hour. Dosages which are particularly preferred are indicated below. The progress can be monitored by a periodic evaluation. The dosages vary, but a preferred dosage for intravenous administration of DNA is approximately 10⁶ to 10¹² copies of the DNA molecule. The compounds of the invention can be administered locally or systemically. The administration is in general carried out parenterally, e.g. intravenously; the DNA can also be administered by directing it to the target site, e.g. by biolistic transfer to an internal or external target site or by a catheter to a site in an artery. The preparation for the parenteral dose comprise sterile aqueous or non-aqueous solutions, suspensions and emulsions. Examples of non-aqueous solvents are propylene glycol, polyethylene glycol, vegetable oils such as olive oil and injectable organic esters such as ethyloleate. Aqueous carriers include water, alcoholic/aqueous solutions, emulsions or suspensions, including saline solution and buffered media. Parenteral vehicles include sodiumchloride solution, Ringer's dextrose solution, dextrose and sodiumchloride, Ringer's lactate solution and solid oils. Intravenous vehicles include liquid supplementary agents and nutrient supplements, electrolyte supplementary solutions (such as those which are based on Ringer's dextrose solution) and the like. Preservatives and other additives such as for example antimicrobial solutions, antioxidants, chelating agents and inert gasses and the like can also be present. Additionally, the pharmaceutical composition of the present invention could comprise protein carriers such as e.g. serum albumin or immunoglobulin, preferably of human origin. Moreover, it is conceivable that the pharmaceutical composition according to the invention could comprise further biologically active agents depending on the intended purpose of the pharmaceutical composition. As already described above, for the treatment of senescence signs, e.g. of the skin, preferably topical forms of administration such as creams, lotions or ointments are selected.

Due to the present invention, it is conceivable that the different IGFBP-2 polynucleotides and IGFBP-2 vectors are administered either alone or in any combination using standard vectors and/or gene transfer systems and optionally in combination with a pharmaceutically acceptable carrier or excipient. After administration, the polynucleotides or vectors can be stably integrated into the patient's genome.

On the other hand, viral vectors can be used which are specific for certain cells or tissues and which persist in the cells. Suitable pharmaceutical carriers and excipients are well-known from the field. The pharmaceutical compositions produced according to the invention can be used for the prevention or treatment or slowing down of different (senescence) diseases which are in particular related to age-induced malignancies.

It is, moreover, possible to use a pharmaceutical composition which is to be administered according to the invention which comprises the IGFBP-2 polynucleotide or the IGFBP-2 vector in gene therapy. Suitable gene transfer systems can inter alia comprise liposomes, receptor-mediated transfer systems, nude DNA and viral vectors such as herpes viruses, retroviruses, adenoviruses and adeno-associated viruses. The transfer of nucleic acids to a specific site in the body for gene therapy can also be effected by using a biolystic transfer system such as the one described by Williams (Proc. Natl. Acad. Sci. USA 88 (1991), 2726-2729. Other methods for the transfer of nucleic acids comprise particle-mediated gene transfer such as e.g. described in Verma, Gene Ther. 15 (1998), 692-699. The prerequisite should be that the introduced polynucleotides and vectors express the gene product after introduction into the cess and preferably remain in this status during the life-span of the cell. For example, cell lines which express the polynucleotide under the control of suitable regulatory sequences can be produced by means of gene technology according to the methods which are known to the skilled person. Host cells can either be transformed on the same plasmid or on separated plasmids with the polypeptide of the invention and a selection marker rather than expression vectors being used which contain viral replication origins. After the introduction of foreign DNA, cells which have been produced by means of gene technology can be left to grow 1 to 2 days in an enriched medium and then a change is made to a selective marker. The selection marker in the recombinant plasmid transfers the selection resistance and allows the selection of cells which have stably integrated the plasmid in their chromosomes and which are left to grow so that they form centers which, in turn, can be cloned and can be dispersed into cell lines.

In a particularly preferred embodiment of the present invention, the pharmaceutical composition is a pharmaceutical composition which is to be administered topically, e.g. as cream, ointment or lotion. The topical administration in form of a cream, lotion or ointment or the like described herein is in particular to be used in the treatment or alleviation of senescence processes of the skin.

The term “for maintenance of tissue and/or organ function” as employed herein relates in particular to the maintenance of a non-diseased state or healthy state of a given organ/a given tissue. This term also comprises the maintenance of said tissue and/or organ in a non-cancerous state or non-tumorous state.

Accordingly, as disclosed herein, IGFBP-2 can medically be used in the prevention, the amelioration and/or the treatment of a proliferative disorder and/or a cancerous disease, in particular cancer. Without being limited, said cancer may be lung cancer, cancer of the reproductive tract, prostate cancer, bone cancer, kidney cancer, cancer of the intestinal tract, stomach cancer or cancer of the supporting or connective tissue. Most preferably, the intestinal tract cancer to be treated or prevented is colon cancer. Corresponding data are also provided in the appended examples.

In a further embodiment, IGFBP-2 can be employed in the maintenance of organ and/or tissue function is the maintenance and/or restoration of body mass and/or body fat. This is in particular desired in the prevention and/or amelioration of cachexia and/or cachexic phenotypes.

Accordingly, IGFBP-2 may also be employed in context of this invention in the medical and/or pharmaceutical invention in patients suffering from cachexia who are cancer patients, AIDS patients, patients suffering from a metabolic disease or patients suffering from an eating disorder, patients suffering from infectious diseases, from psychological disorders as well as from intoxications. Accordingly, also patients suffering from medical and non-medical treatments and being cachexic (e.g. surgical events, therapeutic or accidental irradiation, chemotherapy) may be treated with IGFBP-2 molecules as defined herein.

The term “regulation of senescence processes in cells, tissues and/or organs” comprises in particular the slowing down of senescence processes of the corresponding cells, tissues and/or organs. The pharmaceutical composition described herein which comprises IGFBP-2 molecules is in particular used before or after the first indications of senescence signs in the cells, tissues and/or organs. These indications or signs comprise, without being restricted to those, structural degenerative alterations e.g. of connective tissues or skin, functional losses neuronal tissues including cognitive functions. These indications further include complete loss of regenerative potential leading to impaired tissue- or cell- and tissue-regeneration of e.g. bone mass or stress adaption. Also modification alterations in the DNA sequences or RNA sequences or expressed proteins (e.g. mutations on the nucleic acid sequence level or the protein level) may be an indicative sign of senescence. These sign may, inter alia, be detected by known recombinant or gene detection technologies, like PCR-techniques or protein detection methods like MALDI-TOFF or immunon-detection methods.

In accordance with this invention, in particular human patients should be treated in accordance with the methods and uses provided herein.

A particularly preferred group of patients for the treatment with IGFBP-2 preparations are female patients or female subjects. The uses and therapeutic methods described herein can be used on any subject in need of a corresponding therapy in particular a slowing down of senescence processes. The corresponding therapeutic measures are preferably applied to mammals like dogs, cats, cows, horses, rabbits, apes and most preferably to humans. In order to prevent potential side-effects (e.g. a malignant mechanism), the IGFBP-2 molecule to be administered can be changed in such a way that the malignant potential of IGFBP-2 is eliminated e.g. by a modulation of the cell surface binding of IGFBP-2 or by modulation of the interaction with other components (e.g. IIp45) or other compartments (e.g. cell nucleus). This can be achieved in particular by genetically modified IGFBP-2 variants and/or by supplementation with additional compounds (e.g. small molecules). Alternatively, modulation of IGFBP-2 dependent effects can be achieved by specific manipulation of IGFBP-2 dependent pathways. As an example it might be necessary to have activated the input of IGFBP-2 on the Wnt-signaling pathway, whereas the effect of IGFBP-2 on integrin-signaling (e.g. via FAK-MAP of FAK-PI3-K) is unwanted. In this example the modulation is achieved by use of specific inhibitors (mTOR), rapamycin, MEK1/2, PD98059) or by the activation of specific phosphatases. Such an IGFBP-2 variant in particular comprises the so-called RGE variants where a reduction of the integrin binding was shown. Corresponding examples inter alia comprise the IGFBP-2 molecule encoded by SEQ ID NO:3 and which is shown in SEQ ID NO:4 in the form of its amino acid sequence. Other variants comprise variants with modified proteoglycan interactions. Corresponding variants inter alia comprise the IGFBP-2 molecule encoded by SEQ ID NO:5 whose amino acid sequence is also shown in SEQ ID NO:6. Corresponding other variants are known among the skilled persons (e.g. Jones (1993), PNAS, 90: 10553-10557; Lee (2000), J. Virol., 74: 8867-8875).

In an embodiment of the use according to the invention, the IGFBP-2 polypeptide is selected from the group of
(a) a polypeptide comprising an amino acid sequence as shown in SEQ ID NO:2, 4 or 6;
(b) a polypeptide which is encoded by a nucleic acid as shown in SEQ ID NO:1, 3 or 5; and
(c) a polypeptide fragment or polypeptide derivative of the polypeptides (a) or (b) wherein in this polypeptide derivative one or more amino acid residue(s) is/are conservatively exchanged and wherein this fragment or derivative can effect the same regulation of senescence processes in cells, tissues and/or organs as the polypeptide (a) or (b) and/or wherein this fragment or derivative leads to the same maintenance of tissue and/or organ functions as the polypeptide (a) or (b).

The sequences shown in the appendix provided herein are in particular sequences of the human wild-type IGFBP-2 (SEQ ID NO:1 as encoding sequence with corresponding allelic variants and SEQ ID NO:2 as wild-type amino acid sequence). The sequences are also variants of an IGFBP-2 molecule which can be used according to the invention. These variants are encoded by SEQ ID NO: 3 and 5 and corresponding amino acid sequences are shown in SEQ ID NOs: 4 and 6.

The conservative substitution of one or more amino acid residues in a polypeptide, polypeptide fragment is well-known to the skilled person and comprises inter alia also variants of the IGFBP-2 molecules shown herein, e.g. allelic variants.

The invention moreover comprises the use of IGFBP-2 polypeptides which do not exhibit a large but a sufficient similarity in order to exert one or more functions of the IGFBP-2 described in this invention. According to the invention, similarity is achieved by a conservative substitution of amino acids. Such substitutions comprise the substitution of a certain amino acid in a polypeptide by another amino acid with a comparable characteristic (e.g. chemical properties).

According to Cunningham ((1989), Science, 244, 1081-1085), the conservative amino acid substitutions do not have a phenotypical effect. More in-depth instructions as to which amino acid substitutions have no phenotypical effect can be taken from the literature (e.g.: Bowie (1990), Science, 247, 1306-1310).

Tolerated conservative amino acid substitutions of this invention comprise the substitution of aliphatic or hydrophobic amino acids: Ala, Val, Leu and Ile; moreover, the substitution of the hydroxyl residues of Ser and Thr; the exchange of the acidic groups of Asp and Glu; the substitution of the amide residues of Asn and Gln; the substitution of the basic residues of Lys, Arg and H is; the substitution of the aromatic side chains of Phe, Tyr and Trp and the substitution of small amino acids Ala, Ser, Thr, Met and Gly.

Moreover, the term “conservative amino acid substitution” according to the invention inter alia comprises the amino acid substitutions shown in the table below:

| For Amino Acid | Code | Substitution by one of the group: |
|---|---|---|
| Alanine | A | D-Ala, Gly, beta-Ala, L-Cys, D-Cys |
| Arginine | R | D-Arg, Lys, D-Lys, homo-Arg, D-homo-Arg, |
| | | Met, Ile, D-Met, D-Ile, Orn, D-Orn |
| Aspartamic acid | N | D-Asn, Asp, D-Asp, Glu, D-Glu, Gln, D-Gln |
| Asparaginic Acid | D | D-Asp, D-Asn, Asn, Glu, D-Glu, Gln, D-Gln |
| Cystein | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycin | G | Ala, D-Ala, Pro, D-Pro, β-Ala, Acp |
| Isoleucine | I | D-Ile, Val, D-Val, Leu, D-Leu, Met, D-Met |
| Leucine | L | D-Leu, Val, D-Val, Leu, D-Leu, Met, D-Met |
| Lysine | K | D-Lys, Arg, D-Arg, homo-Arg, D-homo-Arg, |
| | | Met, D-Met, Ile, D-Ile, Orn, D-Orn |
| Methionine | M | D-Met, S-Me-Cys, Ile, D-Ile, Leu, D-Leu, Val, |
| | | D-Val |
| Phenylalanine | F | D-Phe, Tyr, D-Thr, L-Dopa, His, D-His, Trp, |
| | | D-Trp, Trans-3,4, or 5-phenylproline, |
| | | cis-3,4, or 5-phenylproline |
| Proline | P | D-Pro, L-1-thioazolidine-4-carboxylic acid, |
| | | D-or L-1-oxazolidine-4-carboxylic acid |
| Serine | S | D-Ser, Thr, D-Thr, allo-Thr, Met, D-Met, |
| | | Met(O), D-Met(O), L-Cys, D-Cys |
| Threonine | T | D-Thr, Ser, D-Ser, allo-Thr, Met, D-Met, |
| | | Met(O), D-Met(O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met |

Apart from the above-indicated use, such amino acid substitutions possibly increase the stability of the protein or peptide. The invention comprises the use of IGFBP-2 molecules where e.g. one or more peptide bonds in the protein or peptide sequences has/have been substituted by one or more non-peptide bonds. The invention also encompasses substitutions comprising other amino acid residues than the naturally occurring L-amino acids, e.g.: D-amino acids or amino acids, e.g. β- or γ-amino acids which do not occur naturally or which are synthetic.

The identity of such polypeptides with IGFBP-2 molecules as described herein can be calculated by means of the following references: Computational Molecular Biology, Lesk, A. M., ed., Oxford University Press, New York, 1988; Biocomputing: Infoliuaties and Genome Projects, Smith, D M., ed., Academic Press, New York, 1993; Informafies Computer Analysis of Sequence Data, Part 1, Griffin, A.M., and Griffin, H. G., eds., Humana Press, New Jersey, 1994; Sequence Analysis in Molecular Biology, von Heinje, G., Academie Press, 1987; and Sequence Analysis Primer, Gribskov, M. and Devereux, eds., M Stockton Press, New York, 1991.

In connection with this invention, IGFBP-2 fragments or IGFBP-2 derivatives of functional molecules which can also regulate the senescence processes in cells, tissues and/or organs and which in particular can slow down the senescence process or senescence. Corresponding test systems comprise e.g. the production of non-human, transgenic animals expressing these variants, fragments or derivatives. By means of these animals (or their cells, tissues, organs), an increase in the life-span can be measured. Other test systems are described in the experimental part. Functional fragments of IGFBP-2 may, e.g. comprise but are not limited to amino acid sequences as shown in amino acids 28 to 140, 28 to 60, 60 to 80, 80 to 140, 60 to 140, 28 to 80, or 175 to 328 of the amino acid sequence as shown in SEQ ID NO: 2. Again, also these “functional fragments” may comprise additional amino acid sequences (e.g. a fragment defined as 28 to 60 may also comprise 26 to 60, 27 to 60 or 27 to 61 or 26 to 62 and the like). Also comprised in the definition of functional fragments of the IGFBP-2 are nucleic acid molecules encoding the same. These nucleic acid molecules may be comprised in corresponding expression vectors known in the art and described below.

In a further embodiment of the use as described above, nucleic acids are used which encode an IGFBP-2 polypeptide or a functional fragment or derivative thereof. These can in particular be selected from the group
(a) a nucleic acid encoding a polypeptide with the amino acid sequence as shown in SEQ ID NO:2, 4 or 6;
(b) a nucleic acid with the coding sequence as shown in SEQ ID NO:1, 3 or 5;
(c) a nucleic acid encoding a fragment or derivative of a polypeptide which is encoded by a nucleic acid according to (a) or (b), wherein in the derivative one or more amino acid residue(s) is/are conservatively exchanged and wherein the fragment or derivative can effect the same regulation of senescence processes in cells, tissues and/or organs as the polypeptide encoded by (a) or (b) and/or wherein the fragment or derivative leads to the same maintenance of tissue and/or organ functions as the polypeptide encoded by (a) or (b).
(d) a nucleic acid which is at least 80% identical to a polynucleotide as defined in (a) to (c) and which encodes a polypeptide or fragment or derivative thereof which can effect the same regulation of senescence processes in cells, tissues and/or organs as an IGFBP-2 polypeptide and/or which leads to the same maintenance of tissue and/or organ functions; and
(e) a nucleic acid whose complementary strand hybridises to a nucleic acid as defined under (a) to (d) under stringent conditions and encodes a polypeptide or a fragment or derivative thereof which can effect the same regulation of senescence processes in cells, organs and/or tissues as an IGFBP-2 polypeptide and/or which leads to the same maintenance of organ and/or tissue functions.

In a still further embodiment, the present invention thus relates to the use of nucleic acids/polynucleotides which upon expression encode the above-described IGFBP-2 molecules. Concrete, encoding nucleic acid sequences (polynucleotides) are shown in SEQ ID NOs:1, 3 and 5. However, the appendix also provides further encoding nucleic acid sequences by indicating “theoretic nucleic acid sequences”. The use of nucleic acid sequences which are mostly 80%, preferably at least 90% and more preferred at least 95% identical to the sequences indicated in SEQ ID NOs:1, 3 and 5 (or to the sequences shown in the appendix) is also conceivable and envisaged in connection with this invention.

The nucleic acid molecules with at least 80% identity with the sequences shown in SEQ ID NOs:1, 3 and 5 are nucleic acid molecules encoding IGFBP-2 molecules and whose translation product (or transcription product in connection with RNA) leads to a molecule which can exert the function of IGFBP-2 described herein. A corresponding function test are, in particular, transgenic, non-human animals which carry the corresponding nucleic acid molecule as transgene (e.g. knock-in mice). Corresponding examples are given to transgenic mice in the experimental part.

As described above, “functional fragments” of IGFBP-2 may be fragments, like the amino acid stretch from amino acid 28 to 60 or 80 to 140 or 175 to 328 of the sequence as shown in SEQ ID NO: 2. However, these functional fragments may comprise tl-1 amino acid, tl-2 amino acid, tl-3 amino acids, tl-5 amino acids and the like. Also amino acid exchanges within these stretches are envisaged.

The nucleic acids/polynucleotides can be fused with suitable expression control sequences known from the field in order to ensure a suitable transcription and translation of the IGFBP-2 molecule.

The polynucleotide/nucleic acid can e.g. be DNA, cDNA, RNA or synthetically produced DNA or RNA or a recombinantly produced chimeric nucleic acid molecule which comprises each of the polynucleotides either alone or in combination. Preferably, the polynucleotide is part of a vector. Such vectors can also be used in the uses and methods of the invention. Such vectors can comprise further genes such as marker genes which allow the selection of the vector in a suitable host cell and under suitable conditions. Preferably, the polynucleotide of the invention is functionally linked to the expression control sequences which allow the expression in prokaryotic or eukaryotic cells. The expression of the polynucleotides comprises the transcription of the polynucleotide into a translatable mRNA. Regulatory elements which ensure the expression in eukaryotic cells, preferably mammalian cells, are well-known to the skilled person. Commonly, they comprise regulatory sequences which ensure the initiation of the transcription and, optionally, poly-A signals which ensure the termination of the transcription and the stabilisation of the transcript. Additional regulatory elements can comprise transcription and translation enhancers and/or naturally associated or heterologous promotor regions. Possible regulatory elements which allow the expression in prokaryotic host cells comprise e.g. the PI. lac, trp or tac promoter in E. coli and examples of regulatory elements which allow the expression in eukaryotic host cells are the AOX1 or GALL promoter in yeast or the CMV SV40, RSV promoter (Rous Sarcoma Virus) CMV enhancer, SV40 enhancer or a globin intron in mammalian or other animal cells. Apart from elements which are responsible for the initiation of the transcription, such regulatory elements can also comprise transcription termination signals such as the SV40-poly-A site or the tk-poly-A site downstream of the polynucleotide. Moreover, depending on the expression system used, leader sequences which e.g. can secrete the IGFBP-2 into the medium can be added to the coding sequence of the polypeptide which is to be used according to the invention and are known from the field. The leader sequence(s) is/are added to the translation, initiation and termination sequences in the suitable phase and is preferably a leader sequence which can lead the secretion of the translated protein or of a part thereof in the periplasmatic space or the extracellular medium. Optionally, the heterologous sequence can encode a fusion protein, including an N-terminal identification peptide containing the desired features, e.g. the stabilisation or simplified purification of the expressed recombinant IGFBP-2 product; cf. loc. cit. In this context, vectors known from the field such as the Okayama-Berg cDNA expression vector pcDV1 (Pharmacia), pCDM8, pRC/CMV, pcDNA1, pcDNA3 (In-vitrogene) or pSPORT1 (GIBCO BRL) are suitable.

Preferably, the expression control sequences are eukaryotic promoter systems in vectors which can transform transfecting eukaryotic host cells, but control sequences for prokaryotic hosts can also be used. Once the vector was introduced in a suitable host, the host is kept under conditions which are highly suitable for the expression of the nucleotide sequences and the collection and purification of the IGFBP-2 polypeptide which is to be used according to the invention can be carried out as desired.

As described above, the IGFBP-2 molecule (or a functional fragment or derivative or a variant of the molecule) can be used alone or as part of a vector in order to express the IGFBP-2 molecule in cells e.g. for the therapy of senescence diseases or to slow down senescence processes and related diseases. The polynucleotides or vectors containing the DNA sequence(s) which encode one of the polypeptides described above are introduced into the cells which in turn produce the polypeptide of interest. The gene therapy which is based on the introduction of therapeutic genes in cells by ex-vivo or in-vivo methods is one of the most important applications of the gene transfer. Suitable vectors, methods or gene transfer systems for in-vitro or in-vivo gene therapy are described in the literature and are known to the skilled person; cf. e.g. Giordano, Nature Medicine 2 (1996), 534-539; Schaper, Circ. Res. 79 (1996), 911-919; Anderson, Science 256 (1992), 808-813; Verma, Nature 389 (1994), 239; Isner, Lancet 348 (1996), 370-374; Muhlhauser, Circ. Res. 77 (1995), 1077-1086, Onodera, Blood 91 (1998), 30-36; Verma, Gene Ther. 5 (1998), 692-699; Nabel, Ann. N.Y. Acad. Sci. 811 (1997), 289-292; Verzeletti, Hum. Gene Ther. 9 (1998), 2243-51); Wang, Nature Medicine 2 (1996), 714-716; WO 94/29469; WO 97/00957, U.S. Pat. No. 5,580,859; U.S. Pat. No. 5,589,466 or Schaper, Current Opinion in Biotechnology 7 (1996), 635-640 and the documents cited herein. The IGFBP-2 molecules in form or their nucleic acid and vectors can be constructed for the direct introduction or the introduction via liposomes or viral vectors (e.g. adenovirus, retrovirus) into the cell. According to the above, the present invention relates to the use of vectors commonly used in gene technology, in particular plasmids, cosmids, viruses and bacteriophages comprising a polynucleotide which encodes an IGFBP-2 molecule according to the invention. Preferably, the vector is an expression vector and/or a gene transfer or targeting vector. Expression vectors which are derived from viruses such as retroviruses, vaccinia virus, adeno-associated viruses, herpes viruses or bovine papilloma viruses can be used for the transfer of the polynucleotides or vectors of the invention in targeted cell populations. Methods which are well-known to the skilled person can be used for the construction of recombinant vectors; cf. for example the methods described in Sambrook, Molecular Cloning: A Laboratory Manual, Cold Spring Harbor Laboratory (1989) N.Y. and Ausubel, Current Protocols in Molecular Biology, Green Publishing Associates and Wiley Interscience, N.Y. (1989). Alternatively, IGFBP-2-encoding nucleic acids or vectors can be reconstituted in liposomes for transfer in order to target cells. The vectors containing the IGFBP-2 polynucleotides can be transferred into the host cell by well-known methods which vary depending on the kind of cellular host. For example, the calcium chloride transfection is commonly used for prokaryotic cells, while the calcium phosphate treatment or electroporation can be used for other cellular hosts; cf. Sambrook, loc. cit. Once expressed, the polypeptides of the present invention can be purified according to the standard regulations of the field, including ammoniumsulfate precipitation, affinity columns, column chromatography, gel electrophoresis and the like; cf. Scopes, “Protein Purification”, Springer-Verlag, N.Y. (1982). For pharmaceutical purposes, essentially pure polypeptides with a homogeneity of at least approximately 90 to 95% are preferred and 98 to 99% or more homogeneity are most preferred. Once they were purified, in parts or until homogeneity, whatever may be desired, the IGFBP-2 polypeptides can then be therapeutically used (including in an extracorporeal manner).

The invention also relates to the use of a vector containing an IGFBP-2 polynucleotide or an IGFBP-2 nucleic acid, as defined above, for the production of a pharmaceutical composition for the regulation of senescence processes in cells, organs and/or tissues, for the maintenance of organ and/or tissue functions and/or for the treatment or alleviation of senescence symptoms or early senescence. Corresponding vectors are known to the skilled person and have been described above. Particularly preferred vectors are vectors with ubiquitous expression in viruses in prokaryotic and eukaryotic organisms (M13, pSL, pEx, pUC, pBC, pCMV, pBC, pBK, pMSC, PDNR, pLP, pLX, pPROT, pHAT, pRSF, PET, pBA and many others).

Similarly, according to the invention also a host cell can be used which has been genetically modified with a polynucleotide or a nucleic acid, as defined above, or which contains a vector as defined in claim 4 for the production of a pharmaceutical composition for the regulation of senescence processes in cells, tissues and/or organs for the maintenance of tissue and/or organ functions and/or for the treatment or alleviation of senescence symptoms or early senescence.

Examples for corresponding host cells have been mentioned above. However, host cells also comprise E. coli strains, yeasts such as e.g. S. cerevisiae forms or insect cells, mammalian cells and human cells.

The uses as described above, in particular comprise the regulation of senescence processes in cells, organs and/or tissues wherein the regulation is the slowing down of a senescence process in the cells, organs and/or tissues. As a particularly preferred embodiment, the senescence process of inner organs (as described below) and the skin should be slowed down by administration of IGFBP-2 molecules.

The cells, tissues and/or organs whose senescence process is to be slowed down or which are to be maintained in particular in old age are particular derived from the following organs: liver, hear, kidney, lung, brain, peripheral nervous systems (peripheral nerve cells), eyes, ears, stomach, intestine, connective and supportive tissue, bones and skin. Accordingly, the present invention is in particular suitable for the treatment of diseases of these organs.

In a particularly preferred use according to the invention, the cells are skin cells and the organ is the skin. However, in a preferred manner, the present invention also refers to the administration of IGFBP-2 molecules for the regulation of senescence processes and/or the maintenance of the function of the heart and the kidneys. In particular, it will be possible to treat kidney or heart insufficiencies by the administration of IGFBP-2 molecules.

The use of IGFBP-2 molecules as defined herein in the treatment of diseases of nerve cells, the brain or the spinal marrow is also preferred.

According to the present invention, the regulation of senescence processes in cells, organs and/or tissues can lead to a higher resistance against oxidative stress. Accordingly, IGFBP-2 is also used for the prevention, alleviation and/or therapy of diseases due to oxidative stress.

In an embodiment, the invention relates to the use of the above-mentioned IGFBP-2 molecules, vectors or IGFBP-2 expressing host cells for the maintenance of the heart function, the kidney function or the function of the central and/or peripheral nervous system.

Accordingly, the IGFBP-2 molecules, IGFBP-2 vectors or the IGFBP-2 nucleic acids are preferably also used for the maintenance of the organ and/or tissue function in the heart and in particular for the treatment, prevention and/or therapy of a heart disease. The heart diseases can e.g. be a heart insufficiency or a heart attack.

The use of IGFBP-2 molecules, IGFBP-2 vectors or IGFBP-2 nucleic acids also relates to the treatment or alleviation of senescence symptoms or early senescence, the treatment of skin diseases and senescence of the skin, the treatment of a kidney disease, the treatment of a heart disease, the treatment of a disease of the central and/or peripheral nervous system and/or the treatment of a bone disease. The corresponding kidney disease preferably is a kidney insufficiency and the corresponding heart disease preferably is a heart insufficiency. The bone disease also comprises osteoporosis. The disease of the central and/or peripheral nervous system can, inter alia, be a case of Alzheimer's disease, a Parkinson's disease, a dementia, an AIDS dementia, a motor neuron disease, an amyotrophic lateral sclerosis or a neurofibromatosis (Recklinghausen's disease).

The invention also relates to methods of treatment (a) for the treatment and/or alleviation of senescence symptoms, (b) for the treatment of early senescence of cells, tissues and/or organs and/or organisms and/or (c) for the maintenance of tissue and/or organ functions, wherein the method of treatment comprises the administration of a therapeutic amount of an IGFBP-2 molecule as defined in claims 1 to 3, of a vector as defined in claim 4, or a host cell as defined in claim 5 to a patient to be treated. The patient is preferably a mammal and particularly preferred human.

The dosage ranges of an administration of the IGFBP-2 polypeptides, IGFBP-2 polynucleotides and IGFBP-2 vectors are those which are large enough to have the desired effect, where the symptoms of age-induced diseases are improved or where the maintenance of tissue and/or organ functions is achieved In the methods and uses described herein, a mode of action of the IGFBP-2 molecules described herein is independent of the cellular mode of action, i.e. independent of whether the influence on the cells occurs on their surface or even in/at the nuclear compartment. The dosage should, however, not be so high that it causes substantial side-effects such as undesired cross-reactions, anaphylactic reactions and the like. In general, the dose varies according to the age, condition, sex and the extent of the disease in the patient and can be determined by a skilled person. In case of any contraindication, the dosage can be adjusted by the individual physician. It is conceivable that the range of the dose is adjusted to e.g. 0.01 μg to 10 mg of the IGFBP-2 polypeptide. A particular preferred dose is 0.1 μg to 1 mg, still more preferred is 1 μg to 500 μg and most preferred is a dose of 30 μg to 100 μg.

### APPENDIX

In this document, the following sequences are related to SEQ ID NOs:1 to 6 representing IGFBP-2 molecules according to the invention. SEQ ID NOs:1 and 2 relate to human wild-type IGFBP-2; SEQ ID NOs:3 and 4 show an “RGD” mutant variant which does not comprise an integrin binding and which can also preferably be used in the uses and methods of the invention; SEQ ID NOs: 5 and 6 show the also preferred human IGFBP-2 variant which has a mutation at the proteoglycane interaction site.

| SEQ ID NO: 1 | |
|---|---|
| LOCUS | NM_000597 1433 bp mRNA linear PRI 31-OCT.-2000 |
| DEFINITION | Homo sapiens insulin-like growth factor binding protein 2 |
| | (36 kD) (IGFBP2), mRNA. |
| ACCESSION | NM_000597 |
| VERSION | NM_000597.1 GI: 10835156 |
| KEYWORDS | • |
| SOURCE | Homo sapiens (human) |
| ORIGIN | |

| [SEQ ID NO: 1] | |
|---|---|
| 1 | attcggggcg agggaggagg aagaagcgga ggaggcggct cccgctcgca gggccgtgca |
| 61 | cctgcccgcc cgcccgctcg ctcgctcgcc cgccgcgccg cgctgccgac cgccagcatg |
| 121 | ctgccgagag tgggctgccc cgcgctgccg ctgccgccgc cgccgctgct gccgctgctg |
| 181 | ccgctgctgc tgctgctact gggcgcgagt ggcggcggcg gcggggcgcg cgcggaggtg |
| 241 | ctgttccgct gcccgccctg cacacccgag cgcctggccg cctgcgggcc cccgccggtt |
| 301 | gcgccgcccg ccgcggtggc cgcagtggcc ggaggcgccc gcatgccatg cgcggagctc |
| 361 | gtccgggagc cgggctgcgg ctgctgctcg gtgtgcgccc ggctggaggg cgaggcgtgc |
| 421 | ggcgtctaca ccccgcgctg cggccagggg ctgcgctgct atccccaccc gggctccgag |
| 481 | ctgcccctgc aggcgctggt catgggcgag ggcacttgtg agaagcgccg ggacgccgag |
| 541 | tatggcgcca gcccggagca ggttgcagac aatggcgatg accactcaga aggaggcctg |
| 601 | gtggagaacc acgtggacag caccatgaac atgttgggcg ggggaggcag tgctggccgg |
| 661 | aagcccctca agtcgggtat gaaggagctg gccgtgttcc gggagaaggt cactgagcag |
| 721 | caccggcaga tgggcaaggg tggcaagcat caccttggcc tggaggagcc caagaagctg |
| 781 | cgaccacccc ctgccaggac tccctgccaa caggaactgg accaggtcct ggagcggatc |
| 841 | tccaccatgc gccttccgga tgagcggggc cctctggagc acctctactc cctgcacatc |
| 901 | cccaactgtg acaagcatgg cctgtacaac ctcaaacagt gcaagatgtc tctgaacggg |
| 961 | cagcgtgggg agtgctggtg tgtgaacccc aacaccggga agctgatcca gggagccccc |
| 1021 | accatccggg gggaccccga gtgtcatctc ttctacaatg agcagcagga ggcttgcggg |
| 1081 | gtgcacaccc agcggatgca gtagaccgca gccagccggt gcctggcgcc cctgcccccc |
| 1141 | gcccctctcc aaacaccggc agaaaacgga gagtgcttgg gtggtgggtg ctggaggatt |
| 1201 | ttccagttct gacacacgta tttatatttg gaaagagacc agcaccgagc tcggcacctc |
| 1261 | cccggcctct ctcttcccag ctgcagatgc cacacctgct ccttcttgct ttccccgggg |
| 1321 | gaggaagggg gttgtggtcg gggagctggg gtacaggttt ggggaggggg aagagaaatt |
| 1381 | tttatttttg aacccctgtg tcccttttgc ataagattaa aggaaggaaa agt |

IGFBP-2 [Homo sapiens].

Further coding sequences may be deduced with the following table

1-7

| Met | Leu | Pro | Arg | Val | Gly | Cys |
|---|---|---|---|---|---|---|
| AUG 1.00 | CUG 0.83 | CCG 0.77 | CGU 0.74 | GUU 0.51 | GGU 0.59 | UGC 0.51 |
| | CUC 0.07 | CCA 0.15 | CGC 0.25 | GUA 0.26 | GGC 0.38 | UGU 0.49 |
| | CUU 0.04 | CCU 0.08 | CGA 0.01 | GUG 0.16 | GGG 0.02 | |
| | UUG 0.03 | CCC 0.00 | AGG 0.00 | GUC 0.07 | GGA 0.00 | |
| | UUA 0.02 | | AGA 0.00 | | | |
| | CUA 0.00 | | CGG 0.00 | | | |

8-14

| Pro | Ala | Leu | Pro | Leu | Pro | Pro |
|---|---|---|---|---|---|---|
| CCG 0.77 | GCU 0.35 | CUG 0.83 | CCG 0.77 | CUG 0.83 | CCG 0.77 | CCG 0.77 |
| CCA 0.15 | GCA 0.28 | CUC 0.07 | CCA 0.15 | CUC 0.07 | CCA 0.15 | CCA 0.15 |
| CCU 0.08 | GCG 0.26 | CUU 0.04 | CCU 0.08 | CUU 0.04 | CCU 0.08 | CCU 0.08 |
| CCC 0.00 | GCC 0.10 | UUG 0.03 | CCC 0.00 | UUG 0.03 | CCC 0.00 | CCC 0.00 |
| | | UUA 0.02 | | UUA 0.02 | | |
| | | CUA 0.00 | | CUA 0.00 | | |

15-21

| Pro | Pro | Leu | Leu | Pro | Leu | Leu |
|---|---|---|---|---|---|---|
| CCG 0.77 | CCG 0.77 | CUG 0.83 | CUG 0.83 | CCG 0.77 | CUG 0.83 | CUG 0.83 |
| CCA 0.15 | CCA 0.15 | CUC 0.07 | CUC 0.07 | CCA 0.15 | CUC 0.07 | CUC 0.07 |
| CCU 0.08 | CCU 0.08 | CUU 0.04 | CUU 0.04 | CCU 0.08 | CUU 0.04 | CUU 0.04 |
| CCC 0.00 | CCC 0.00 | UUG 0.03 | UUG 0.03 | CCC 0.00 | UUG 0.03 | UUG 0.03 |
| | | UUA 0.02 | UUA 0.02 | | UUA 0.02 | UGA 0.02 |
| | | CUA 0.00 | CUA 0.00 | | CUA 0.00 | CUA 0.00 |

22-28

| Pro | Leu | Leu | Leu | Leu | Leu | Leu |
|---|---|---|---|---|---|---|
| CCG 0.77 | CUG 0.83 | CUG 0.83 | CUG 0.83 | CUG 0.83 | CUG 0.83 | CUG 0.83 |
| CCA 0.15 | CUC 0.07 | CUC 0.07 | CUC 0.07 | CUC 0.07 | CUC 0.07 | CUC 0.07 |
| CCU 0.08 | CUU 0.04 | CUU 0.04 | CUU 0.04 | CUU 0.04 | CUU 0.04 | CUU 0.04 |
| CCC 0.00 | UUG 0.03 | UUG 0.03 | UUG 0.03 | UUG 0.03 | UUG 0.03 | UUG 0.03 |
| | UUA 0.02 | UUA 0.02 | UUA 0.02 | UUA 0.02 | UUA 0.02 | UUA 0.02 |
| | CUA 0.00 | CUA 0.00 | CUA 0.00 | CUA 0.00 | CUA 0.00 | CUA 0.00 |

29-35

| Gly | Ala | Ser | Gly | Gly | Gly | Gly |
|---|---|---|---|---|---|---|
| GGU 0.59 | GCU 0.35 | UCC 0.37 | GGU 0.59 | GGU 0.59 | GGU 0.59 | GGU 0.59 |
| GGC 0.38 | GCA 0.28 | UCU 0.34 | GGC 0.38 | GGC 0.38 | GGC 0.38 | GGC 0.38 |
| GGG 0.02 | GCG 0.26 | AGC 0.20 | GGG 0.02 | GGG 0.02 | GGG 0.02 | GGG 0.02 |
| GGA 0.00 | GCC 0.10 | UCG 0.04 | GGA 0.00 | GGA 0.00 | GGA 0.00 | GGA 0.00 |
| | | AGU 0.03 | | | | |
| | | UCA 0.02 | | | | |

36-42

| Gly | Ala | Arg | Ala | Glu | Val | Leu |
|---|---|---|---|---|---|---|
| GGU 0.59 | GCU 0.35 | CGU 0.74 | GCU 0.35 | GAA 0.78 | GUU 0.51 | CUG 0.83 |
| GGC 0.38 | GCA 0.28 | CGC 0.25 | GCA 0.28 | GAG 0.22 | GUA 0.26 | CUC 0.07 |
| GGG 0.02 | GCG 0.26 | CGA 0.01 | GCG 0.26 | | GUG 0.16 | CUU 0.04 |
| GGA 0.00 | GCC 0.10 | AGG 0.00 | GCC 0.10 | | GUC 0.07 | UUG 0.03 |
| | | AGA 0.00 | | | | UUA 0.02 |
| | | CGG 0.00 | | | | CUA 0.00 |

43-49

| Phe | Arg | Cys | Pro | Pro | Cys | Thr |
|---|---|---|---|---|---|---|
| UUC 0.76 | CGU 0.74 | UGC 0.51 | CCG 0.77 | CCG 0.77 | UGC 0.51 | ACC 0.55 |
| UUU 0.24 | CGC 0.25 | UGU 0.49 | CCA 0.15 | CCA 0.15 | UGU 0.49 | ACU 0.35 |
| | CGA 0.01 | | CCU 0.08 | CCU 0.08 | | ACG 0.07 |
| | AGG 0.00 | | CCC 0.00 | CCC 0.00 | | ACA 0.04 |
| | AGA 0.00 | | | | | |
| | CGG 0.00 | | | | | |

50-56

| Pro | Glu | Arg | Leu | Ala | Ala | Cys |
|---|---|---|---|---|---|---|
| CCG 0.77 | GAA 0.78 | CGU 0.74 | CUG 0.83 | GCU 0.35 | GCU 0.35 | UGC 0.51 |
| CCA 0.15 | GAG 0.22 | CGC 0.25 | CUC 0.07 | GCA 0.28 | GCA 0.28 | UGU 0.49 |
| CCU 0.08 | | CGA 0.01 | CUU 0.04 | GCG 0.26 | GCG 0.26 | |
| CCC 0.00 | | AGG 0.00 | UUG 0.03 | GCC 0.10 | GCC 0.10 | |
| | | AGA 0.00 | UUA 0.02 | | | |
| | | CGG 0.00 | CUA 0.00 | | | |

57-63

| Gly | Pro | Pro | Pro | Val | Ala | Pro |
|---|---|---|---|---|---|---|
| GGU 0.59 | CCG 0.77 | CCG 0.77 | CCG 0.77 | GUU 0.51 | GCU 0.35 | CCG 0.77 |
| GGC 0.38 | CCA 0.15 | CCA 0.15 | CCA 0.15 | GUA 0.26 | GCA 0.28 | CCA 0.15 |
| GGG 0.02 | CCU 0.08 | CCU 0.08 | CCU 0.08 | GUG 0.16 | GCG 0.26 | CCU 0.08 |
| GGA 0.00 | CCC 0.00 | CCC 0.00 | CCC 0.00 | GUC 0.07 | GCC 0.10 | CCC 0.00 |

64-70

| Pro | Ala | Ala | Val | Ala | Ala | Val |
|---|---|---|---|---|---|---|
| CCG 0.77 | GCU 0.35 | GCU 0.35 | GUU 0.51 | GCU 0.35 | GCU 0.35 | GUU 0.51 |
| CCA 0.15 | GCA 0.28 | GCA 0.28 | GUA 0.26 | GCA 0.28 | GCA 0.28 | GUA 0.26 |
| CCU 0.08 | GCG 0.26 | GCG 0.26 | GUG 0.16 | GCG 0.26 | GCG 0.26 | GUG 0.16 |
| CCC 0.00 | GCC 0.10 | GCC 0.10 | GUC 0.07 | GCC 0.10 | GCC 0.10 | GUC 0.07 |

71-77

| Ala | Gly | Gly | Ala | Arg | Met | Pro |
|---|---|---|---|---|---|---|
| GCU 0.35 | GGU 0.59 | GGU 0.59 | GCU 0.35 | CGU 0.74 | AUG 1.00 | CCG 0.77 |
| GCA 0.28 | GGC 0.38 | GGC 0.38 | GCA 0.28 | CGC 0.25 | | CCA 0.15 |
| GCG 0.26 | GGG 0.02 | GGG 0.02 | GCG 0.26 | CGA 0.01 | | CCU 0.08 |
| GCC 0.10 | GGA 0.00 | GGA 0.00 | GCC 0.10 | AGG 0.00 | | CCC 0.00 |
| | | | | AGA 0.00 | | |
| | | | | CGG 0.00 | | |

78-84

| Cys | Ala | Glu | Leu | Val | Arg | Glu |
|---|---|---|---|---|---|---|
| UGC 0.51 | GCU 0.35 | GAA 0.78 | CUG 0.83 | GUU 0.51 | CGU 0.74 | GAA 0.78 |
| UGU 0.49 | GCA 0.28 | GAG 0.22 | CUC 0.07 | GUA 0.26 | CGC 0.25 | GAG 0.22 |
| | GCG 0.26 | | CUU 0.04 | GUG 0.16 | CGA 0.01 | |
| | GCC 0.10 | | UUG 0.03 | GUC 0.07 | AGG 0.00 | |
| | | | UUA 0.02 | | AGA 0.00 | |
| | | | CUA 0.00 | | CGG 0.00 | |

85-91

| Pro | Gly | Cys | Gly | Cys | Cys | Ser |
|---|---|---|---|---|---|---|
| CCG 0.77 | GGU 0.59 | UGC 0.51 | GGU 0.59 | UGC 0.51 | UGC 0.51 | UCC 0.37 |
| CCA 0.15 | GGC 0.38 | UGU 0.49 | GGC 0.38 | UGU 0.49 | UGU 0.49 | UCU 0.34 |
| CCU 0.08 | GGG 0.02 | | GGG 0.02 | | | AGC 0.20 |
| CCC 0.00 | GGA 0.00 | | GGA 0.00 | | | UCG 0.04 |
| | | | | | | AGU 0.03 |
| | | | | | | UCA 0.02 |

92-98

| Val | Cys | Ala | Arg | Leu | Glu | Gly |
|---|---|---|---|---|---|---|
| GUU 0.51 | UGC 0.51 | GCU 0.35 | CGU 0.74 | CUG 0.83 | GAA 0.78 | GGU 0.59 |
| GUA 0.26 | UGU 0.49 | GCA 0.28 | CGC 0.25 | CUC 0.07 | GAG 0.22 | GGC 0.38 |
| GUG 0.16 | | GCG 0.26 | CGA 0.01 | CUU 0.04 | | GGG 0.02 |
| GUC 0.07 | | GCC 0.10 | AGG 0.00 | UUG 0.03 | | GGA 0.00 |
| | | | AGA 0.00 | UUA 0.02 | | |
| | | | CGG 0.00 | CUA 0.00 | | |

99-105

| Glu | Ala | Cys | Gly | Val | Tyr | Thr |
|---|---|---|---|---|---|---|
| GAA 0.78 | GCU 0.35 | UGC 0.51 | GGU 0.59 | GUU 0.51 | UAC 0.75 | ACC 0.55 |
| GAG 0.22 | GCA 0.28 | UGU 0.49 | GGC 0.38 | GUA 0.26 | UAU 0.25 | ACU 0.35 |
| | GCG 0.26 | | GGG 0.02 | GUG 0.16 | | ACG 0.07 |
| | GCC 0.10 | | GGA 0.00 | GUC 0.07 | | ACA 0.04 |

106-112

| Pro | Arg | Cys | Gly | Gln | Gly | Leu |
|---|---|---|---|---|---|---|
| CCG 0.77 | CGU 0.74 | UGC 0.51 | GGU 0.59 | CAG 0.86 | GGU 0.59 | CUG 0.83 |
| CCA 0.15 | CGC 0.25 | UGU 0.49 | GGC 0.38 | CAA 0.14 | GGC 0.38 | CUC 0.07 |
| CCU 0.08 | CGA 0.01 | | GGG 0.02 | | GGG 0.02 | CUU 0.04 |
| CCC 0.00 | AGG 0.00 | | GGA 0.00 | | GGA 0.00 | UUG 0.03 |
| | AGA 0.00 | | | | | UUA 0.02 |
| | CGG 0.00 | | | | | CUA 0.00 |

113-119

| Arg | Cys | Tyr | Pro | His | Pro | Gly |
|---|---|---|---|---|---|---|
| CGU 0.74 | UGC 0.51 | UAC 0.75 | CCG 0.77 | CAC 0.83 | CCG 0.77 | GGU 0.59 |
| CGC 0.25 | UGU 0.49 | UAU 0.25 | CCA 0.15 | CAU 0.17 | CCA 0.15 | GGC 0.38 |
| CGA 0.01 | | | CCU 0.08 | | CCU 0.08 | GGG 0.02 |
| AGG 0.00 | | | CCC 0.00 | | CCC 0.00 | GGA 0.00 |
| AGA 0.00 | | | | | | |
| CGG 0.00 | | | | | | |

120-126

| Ser | Glu | Leu | Pro | Leu | Gln | Ala |
|---|---|---|---|---|---|---|
| UCC 0.37 | GAA 0.78 | CUG 0.83 | CCG 0.77 | CUG 0.83 | CAG 0.86 | GCU 0.35 |
| UCU 0.34 | GAG 0.22 | CUC 0.07 | CCA 0.15 | CUC 0.07 | CAA 0.14 | GCA 0.28 |
| AGC 0.20 | | CUU 0.04 | CCU 0.08 | CUU 0.04 | | GCG 0.26 |
| UCG 0.04 | | UUG 0.03 | CCC 0.00 | UUG 0.03 | | GCC 0.10 |
| AGU 0.03 | | UUA 0.02 | | UUA 0.02 | | |
| UCA 0.02 | | CUA 0.00 | | CUA 0.00 | | |

127-133

| Leu | Val | Met | Gly | Glu | Gly | Thr |
|---|---|---|---|---|---|---|
| CUG 0.83 | GUU 0.51 | AUG 1.00 | GGU 0.59 | GAA 0.78 | GGU 0.59 | ACC 0.55 |
| CUC 0.07 | GUA 0.26 | | GGC 0.38 | GAG 0.22 | GGC 0.38 | ACU 0.35 |
| CUU 0.04 | GUG 0.16 | | GGG 0.02 | | GGG 0.02 | ACG 0.07 |
| UUG 0.03 | GUC 0.07 | | GGA 0.00 | | GGA 0.00 | ACA 0.04 |
| UUA 0.02 | | | | | | |
| CUA 0.00 | | | | | | |

134-140

| Cys | Glu | Lys | Arg | Arg | Asp | Ala |
|---|---|---|---|---|---|---|
| UGC 0.51 | GAA 0.78 | AAA 0.74 | CGU 0.74 | CGU 0.74 | GAC 0.67 | GCU 0.35 |
| UGU 0.49 | GAG 0.22 | AAG 0.26 | CGC 0.25 | CGC 0.25 | GAU 0.33 | GCA 0.28 |
| | | | CGA 0.01 | CGA 0.01 | | GCG 0.26 |
| | | | AGG 0.00 | AGG 0.00 | | GCC 0.10 |
| | | | AGA 0.00 | AGA 0.00 | | |
| | | | CGG 0.00 | CGG 0.00 | | |
| 218 | 316 | 272 | 128 | 135 | 137 | 121 |

141-147

| Glu | Tyr | Gly | Ala | Ser | Pro | Glu |
|---|---|---|---|---|---|---|
| GAA 0.78 | UAC 0.75 | GGU 0.59 | GCU 0.35 | UCC 0.37 | CCG 0.77 | GAA 0.78 |
| GAG 0.22 | UAU 0.25 | GGC 0.38 | GCA 0.28 | UCU 0.34 | CCA 0.15 | GAG 0.22 |
| | | GGG 0.02 | GCG 0.26 | AGC 0.20 | CCU 0.08 | |
| | | GGA 0.00 | GCC 0.10 | UCG 0.04 | CCC 0.00 | |
| | | | | AGU 0.03 | | |
| | | | | UCA 0.02 | | |

148-154

| Gln | Val | Ala | Asp | Asn | Gly | Asp |
|---|---|---|---|---|---|---|
| CAG 0.86 | GUU 0.51 | GCU 0.35 | GAC 0.67 | AAC 0.94 | GGU 0.59 | GAC 0.67 |
| CAA 0.14 | GUA 0.26 | GCA 0.28 | GAU 0.33 | AAU 0.06 | GGC 0.38 | GAG 0.33 |
| | GUG 0.16 | GCG 0.26 | | | GGG 0.02 | |
| | GUC 0.07 | GCC 0.10 | | | GGA 0.00 | |

155-161

| Asp | His | Ser | Glu | Gly | Gly | Leu |
|---|---|---|---|---|---|---|
| GAC 0.67 | CAC 0.83 | UCC 0.37 | GAA 0.78 | GGU 0.59 | GGU 0.59 | CUG 0.83 |
| GAU 0.33 | CAU 0.17 | UCU 0.34 | GAG 0.22 | GGC 0.38 | GGC 0.38 | CUC 0.07 |
| | | AGC 0.20 | | GGG 0.02 | GGG 0.02 | CUU 0.04 |
| | | UCG 0.04 | | GGA 0.00 | GGA 0.00 | UUG 0.03 |
| | | AGU 0.03 | | | UUA 0.02 | |
| | | UCA 0.02 | | | CUA 0.00 | |

162-168

| Val | Glu | Asn | His | Val | Asp | Ser |
|---|---|---|---|---|---|---|
| GUU 0.51 | GAA 0.78 | AAC 0.94 | CAC 0.83 | GUU 0.51 | GAC 0.67 | UCC 0.37 |
| GUA 0.26 | GAG 0.22 | AAU 0.06 | CAU 0.17 | GUA 0.26 | GAU 0.33 | UCU 0.34 |
| GUG 0.16 | | | | GUG 0.16 | | AGC 0.20 |
| GUC 0.07 | | | | GUC 0.07 | | UCG 0.04 |
| | | | | | | AGU 0.03 |
| | | | | | | UCA 0.02 |

169-175

| Thr | Met | Asn | Met | Leu | Gly | Gly |
|---|---|---|---|---|---|---|
| ACC 0.55 | AUG 1.00 | AAC 0.94 | AUG 1.00 | CUG 0.83 | GGU 0.59 | GGU 0.59 |
| ACU 0.35 | | AAU 0.06 | | CUC 0.07 | GGC 0.38 | GGC 0.38 |
| ACG 0.07 | | | | CUU 0.04 | GGG 0.02 | GGG 0.02 |
| ACA 0.04 | | | | UUG 0.03 | GGA 0.00 | GGA 0.00 |
| | | | | UUA 0.02 | | |
| | | | | CUA 0.00 | | |

176-182

| Gly | Gly | Ser | Ala | Gly | Arg | Lys |
|---|---|---|---|---|---|---|
| GGU 0.59 | GGU 0.59 | UCC 0.37 | GCU 0.35 | GGU 0.59 | CGU 0.74 | AAA 0.74 |
| GGC 0.38 | GGC 0.38 | UCU 0.34 | GCA 0.28 | GGC 0.38 | CGC 0.25 | AAG 0.26 |
| GGG 0.02 | GGG 0.02 | AGC 0.20 | GCG 0.26 | GGG 0.02 | CGA 0.01 | |
| GGA 0.00 | GGA 0.00 | UCG 0.04 | GCC 0.10 | GGA 0.00 | AGG 0.00 | |
| | | AGU 0.03 | | | AGA 0.00 | |
| | | UCA 0.02 | | | CGG 0.00 | |

183-189

| Pro | Leu | Lys | Ser | Gly | Met | Lys |
|---|---|---|---|---|---|---|
| CCG 0.77 | CUG 0.83 | AAA 0.74 | UCC 0.37 | GGU 0.59 | AUG 1.00 | AAA 0.74 |
| CCA 0.15 | CUC 0.07 | AAG 0.26 | UCU 0.34 | GGC 0.38 | | AAG 0.26 |
| CCU 0.08 | CUU 0.04 | | AGC 0.20 | GGG 0.02 | | |
| CCC 0.00 | UUG 0.03 | | UCG 0.04 | GGA 0.00 | | |
| | UUA 0.02 | | AGU 0.03 | | | |
| | CUA 0.00 | | UCA 0.02 | | | |

190-196

| Glu | Leu | Ala | Val | Phe | Arg | Glu |
|---|---|---|---|---|---|---|
| GAA 0.78 | CUG 0.83 | GCU 0.35 | GUU 0.51 | UUC 0.76 | CGU 0.74 | GAA 0.78 |
| GAG 0.22 | CUC 0.07 | GCA 0.28 | GUA 0.26 | UUU 0.24 | CGC 0.25 | GAG 0.22 |
| | CUU 0.04 | GCG 0.26 | GUG 0.16 | | CGA 0.01 | |
| | UUG 0.03 | GCC 0.10 | GUC 0.07 | | AGG 0.00 | |
| | UUA 0.02 | | | | AGA 0.00 | |
| | CUA 0.00 | | | | CGG 0.00 | |

197-203

| Lys | Val | Thr | Glu | Gln | His | Arg |
|---|---|---|---|---|---|---|
| AAA 0.74 | GUU 0.51 | ACC 0.55 | GAA 0.78 | CAG 0.86 | CAC 0.83 | CGU 0.74 |
| AAG 0.26 | GUA 0.26 | ACU 0.35 | GAG 0.22 | CAA 0.14 | CAU 0.17 | CGC 0.25 |
| | GUG 0.16 | ACG 0.07 | | | | CGA 0.01 |
| | GUC 0.07 | ACA 0.04 | | | | AGG 0.00 |
| | | | | | | AGA 0.00 |
| | | | | | | CGG 0.00 |

204-210

| Gln | Met | Gly | Lys | Gly | Gly | Lys |
|---|---|---|---|---|---|---|
| CAG 0.86 | AUG 1.00 | GGU 0.59 | AAA 0.74 | GGU 0.59 | GGU 0.59 | AAA 0.74 |
| CAA 0.14 | | GGC 0.38 | AAG 0.26 | GGC 0.38 | GGC 0.38 | AAG 0.26 |
| | | GGG 0.02 | | GGG 0.02 | GGG 0.02 | |
| | | GGA 0.00 | | GGA 0.00 | GGA 0.00 | |

211-217

| His | His | Leu | Gly | Leu | Glu | Glu |
|---|---|---|---|---|---|---|
| CAC 0.83 | CAC 0.83 | CUG 0.83 | GGU 0.59 | CUG 0.83 | GAA 0.78 | GAA 0.78 |
| CAU 0.17 | CAU 0.17 | CUC 0.07 | GGC 0.38 | CUC 0.07 | GAG 0.22 | GAG 0.22 |
| | | CUU 0.04 | GGG 0.02 | CUU 0.04 | | |
| | | UUG 0.03 | GGA 0.00 | UUG 0.03 | | |
| | | UUA 0.02 | | UUA 0.02 | | |
| | | CUA 0.00 | | CUA 0.00 | | |

218-224

| Pro | Lys | Lys | Leu | Arg | Pro | Pro |
|---|---|---|---|---|---|---|
| CCG 0.77 | AAA 0.74 | AAA 0.74 | CUG 0.83 | CGU 0.74 | CCG 0.77 | CCG 0.77 |
| CCA 0.15 | AAG 0.26 | AAG 0.26 | CUC 0.07 | CGC 0.25 | CCA 0.15 | CCA 0.15 |
| CCU 0.08 | | | CUU 0.04 | CGA 0.01 | CCU 0.08 | CCU 0.08 |
| CCC 0.00 | | | UUG 0.03 | AGG 0.00 | CCC 0.00 | CCC 0.00 |
| | | | UUA 0.02 | AGA 0.00 | | |
| | | | CUA 0.00 | CGG 0.00 | | |

225-231

| Pro | Ala | Arg | Thr | Pro | Cys | Gln |
|---|---|---|---|---|---|---|
| CCG 0.77 | GCU 0.35 | CGU 0.74 | ACC 0.55 | CCG 0.77 | UGC 0.51 | CAG 0.86 |
| CCA 0.15 | GCA 0.28 | CGC 0.25 | ACU 0.35 | CCA 0.15 | UGU 0.49 | CAA 0.14 |
| CCU 0.08 | GCG 0.26 | CGA 0.01 | ACG 0.07 | CCU 0.08 | | |
| CCC 0.00 | GCC 0.10 | AGG 0.00 | ACA 0.04 | CCC 0.00 | | |
| | | AGA 0.00 | | | | |
| | | CGG 0.00 | | | | |

232-238

| Gln | Glu | Leu | Asp | Gln | Val | Leu |
|---|---|---|---|---|---|---|
| CAG 0.86 | GAA 0.78 | CUG 0.83 | GAC 0.67 | CAG 0.86 | GUU 0.51 | CUG 0.83 |
| CAA 0.14 | GAG 0.22 | CUC 0.07 | GAU 0.33 | CAA 0.14 | GUA 0.26 | CUC 0.07 |
| | | CUU 0.04 | | | GUG 0.16 | CUU 0.04 |
| | | UUG 0.03 | | | GUC 0.07 | UUG 0.03 |
| | | UUA 0.02 | | | | UUA 0.02 |
| | | CUA 0.00 | | | | CUA 0.00 |

239-245

| Glu | Arg | Ile | Ser | Thr | Met | Arg |
|---|---|---|---|---|---|---|
| GAA 0.78 | CGU 0.74 | AUC 0.83 | UCC 0.37 | ACC 0.55 | AUG 1.00 | CGU 0.74 |
| GAG 0.22 | CGC 0.25 | AUU 0.17 | UCU 0.34 | ACU 0.35 | | CGC 0.25 |
| | CGA 0.01 | AUA 0.00 | AGC 0.20 | ACG 0.07 | | CGA 0.01 |
| | AGG 0.00 | | UCG 0.04 | ACA 0.04 | | AGG 0.00 |
| | AGA 0.00 | | AGU 0.03 | | | AGA 0.00 |
| | CGG 0.00 | | UCA 0.02 | | | CGG 0.00 |

246-252

| Leu | Pro | Asp | Glu | Arg | Gly | Pro |
|---|---|---|---|---|---|---|
| CUG 0.83 | CCG 0.77 | GAC 0.67 | GAA 0.78 | CGU 0.74 | GGU 0.59 | CCG 0.77 |
| CUC 0.07 | CCA 0.15 | GAU 0.33 | GAG 0.22 | CGC 0.25 | GGC 0.38 | CCA 0.15 |
| CUU 0.04 | CCU 0.08 | | | CGA 0.01 | GGG 0.02 | CCU 0.08 |
| UUG 0.03 | CCC 0.00 | | | AGG 0.00 | GGA 0.00 | CCC 0.00 |
| UUA 0.02 | | | | AGA 0.00 | | |
| CUA 0.00 | | | | CGG 0.00 | | |

253-259

| Leu | Glu | His | Leu | Tyr | Ser | Leu |
|---|---|---|---|---|---|---|
| CUG 0.83 | GAA 0.78 | CAC 0.83 | CUG 0.83 | UAC 0.75 | UCC 0.37 | CUG 0.83 |
| CUC 0.07 | GAG 0.22 | CAU 0.17 | CUC 0.07 | UAU 0.25 | UCU 0.34 | CUC 0.07 |
| CUU 0.04 | | | CUU 0.04 | | AGC 0.20 | CUU 0.04 |
| UUG 0.03 | | | UUG 0.03 | | UCG 0.04 | UUG 0.03 |
| UUA 0.02 | | | UUA 0.02 | | AGU 0.03 | UUA 0.02 |
| CUA 0.00 | | | CUA 0.00 | | UCA 0.02 | CUA 0.00 |

260-266

| His | Ile | Pro | Asn | Cys | Asp | Lys |
|---|---|---|---|---|---|---|
| CAC 0.83 | AUC 0.83 | CCG 0.77 | AAC 0.94 | UGC 0.51 | GAC 0.67 | AAA 0.74 |
| CAU 0.17 | AUU 0.17 | CCA 0.15 | AAU 0.06 | UGU 0.49 | GAU 0.33 | AAG 0.26 |
| | AUA 0.00 | CCU 0.08 | | | | |
| | | CCC 0.00 | | | | |

267-273

| His | Gly | Leu | Tyr | Asn | Leu | Lys |
|---|---|---|---|---|---|---|
| CAC 0.83 | GGU 0.59 | CUG 0.83 | UAC 0.75 | AAC 0.94 | CUG 0.83 | AAA 0.74 |
| CAU 0.17 | GGC 0.38 | CUC 0.07 | UAU 0.25 | AAU 0.06 | CUC 0.07 | AAG 0.26 |
| | GGG 0.02 | CUU 0.04 | | | CUU 0.04 | |
| | GGA 0.00 | UUG 0.03 | | | UUG 0.03 | |
| | | UUA 0.02 | | | UUA 0.02 | |
| | | CUA 0.00 | | | CUA 0.00 | |

274-280

| Gln | Cys | Lys | Met | Ser | Leu | Asn |
|---|---|---|---|---|---|---|
| CAG 0.86 | UGC 0.51 | AAA 0.74 | AUG 1.00 | UCC 0.37 | CUG 0.83 | AAC 0.94 |
| CAA 0.14 | UGU 0.49 | AAG 0.26 | | UCU 0.34 | CUC 0.07 | AAU 0.06 |
| | | | | AGC 0.20 | CUU 0.04 | |
| | | | | UCG 0.04 | UUG 0.03 | |
| | | | | AGU 0.03 | UUA 0.02 | |
| | | | | UCA 0.02 | CUA 0.00 | |

281-287

| Gly | Gln | Arg | Gly | Glu | Cys | Trp |
|---|---|---|---|---|---|---|
| GGU 0.59 | CAG 0.86 | CGU 0.74 | GGU 0.59 | GAA 0.78 | UGC 0.51 | UGG 1.00 |
| GGC 0.38 | CAA 0.14 | CGC 0.25 | GGC 0.38 | GAG 0.22 | UGU 0.49 | |
| GGG 0.02 | | CGA 0.01 | GGG 0.02 | | | |
| GGA 0.00 | | AGG 0.00 | GGA 0.00 | | | |
| | | AGA 0.00 | | | | |
| | | CGG 0.00 | | | | |

288-294

| Cys | Val | Asn | Pro | Asn | Thr | Gly |
|---|---|---|---|---|---|---|
| UGC 0.51 | GUU 0.51 | AAC 0.94 | CCG 0.77 | AAC 0.94 | ACC 0.55 | GGU 0.59 |
| UGU 0.49 | GUA 0.26 | AAU 0.06 | CCA 0.15 | AAU 0.06 | ACU 0.35 | GGC 0.38 |
| | GUG 0.16 | | CCU 0.08 | | ACG 0.07 | GGG 0.02 |
| | GUC 0.07 | | CCC 0.00 | | ACA 0.04 | GGA 0.00 |

295-301

| Lys | Leu | Ile | Gln | Gly | Ala | Pro |
|---|---|---|---|---|---|---|
| AAA 0.74 | CUG 0.83 | AUC 0.83 | CAG 0.86 | GGU 0.59 | GCU 0.35 | CCG 0.77 |
| AAG 0.26 | CUC 0.07 | AUU 0.17 | CAA 0.14 | GGC 0.38 | GCA 0.28 | CCA 0.15 |
| | CUU 0.04 | AUA 0.00 | | GGG 0.02 | GCG 0.26 | CCU 0.08 |
| | UUG 0.03 | | | GGA 0.00 | GCC 0.10 | CCC 0.00 |
| | UUA 0.02 | | | | | |
| | CUA 0.00 | | | | | |

302-308

| Thr | Ile | Arg | Gly | Asp | Pro | Glu |
|---|---|---|---|---|---|---|
| ACC 0.55 | AUC 0.83 | CGU 0.74 | GGU 0.59 | GAC 0.67 | CCG 0.77 | GAA 0.78 |
| ACU 0.35 | AUU 0.17 | CGC 0.25 | GGC 0.38 | GAU 0.33 | CCA 0.15 | GAG 0.22 |
| ACG 0.07 | AUA 0.00 | CGA 0.01 | GGG 0.02 | | CCU 0.08 | |
| ACA 0.04 | | AGG 0.00 | GGA 0.00 | | CCC 0.00 | |
| | | AGA 0.00 | | | | |
| | | CGG 0.00 | | | | |

309-315

| Cys | His | Leu | Phe | Tyr | Asn | Glu |
|---|---|---|---|---|---|---|
| UGC 0.51 | CAC 0.83 | CUG 0.83 | UUC 0.76 | UAC 0.75 | AAC 0.94 | GAA 0.78 |
| UGU 0.49 | CAU 0.17 | CUC 0.07 | UUU 0.24 | UAU 0.25 | AAU 0.06 | GAG 0.22 |
| | | CUU 0.04 | | | | |
| | | UUG 0.03 | | | | |
| | | UUA 0.02 | | | | |
| | | CUA 0.00 | | | | |

316-322

| Gln | Gln | Glu | Ala | Cys | Gly | Val |
|---|---|---|---|---|---|---|
| CAG 0.86 | CAG 0.86 | GAA 0.78 | GCU 0.35 | UGC 0.51 | GGU 0.59 | GUU 0.51 |
| CAA 0.14 | CAA 0.14 | GAG 0.22 | GCA 0.28 | UGU 0.49 | GGC 0.38 | GUA 0.26 |
| | | | GCG 0.26 | | GGG 0.02 | GUG 0.16 |
| | | | GCC 0.10 | | GGA 0.00 | GUC 0.07 |

323-328

| | | | | | |
|---|---|---|---|---|---|
| His | Thr | Gln | Arg | Met | Gln |
| CAC 0.83 | ACC 0.55 | CAG 0.86 | CGU 0.74 | AUG 1.00 | CAG 0.86 |
| CAU 0.17 | ACU 0.35 | CAA 0.14 | CGC 0.25 | | CAA 0.14 |
| | ACG 0.07 | | CGA 0.01 | | |
| | ACA 0.04 | | AGG 0.00 | | |
| | | | AGA 0.00 | | |
| | | | CGG 0.00 | | |

| SEQ ID NO: 2 | |
|---|---|
| Amino acid sequence of human IGFBP-2 | |
| IGFBP-2 [Homo sapiens]. | |
| ACCESSION NP_000588 | |
| 1 | mlprvgcpal plppppllpl lpllllllga sgggggarae vlfrcppctp |
| 51 | erlaacgppp vappaavaav aggarmpcae lvrepgcgcc svcarlegea |
| 101 | cgvytprcgq glrcyphpgs elplqalvmg egtcekrrda eygaspeqva |
| 151 | dngddhsegg lvenhvdstm nmlggggsag rkplksgmke lavfrekvte |
| 201 | qhrqmgkggk hhlgleepkk lrpppartpc qqeldqvler istmrlpder |
| 251 | gplehlyslh ipncdkhgly nlkqckmsln gqrgecwcvn pntgkliqga |
| 301 | ptirgdpech lfyneqqeac gvhtqrmq |

| SEQ ID NO: 3 | |
|---|---|
| RGE-IGFBP-2 [Homo Sapiens] | |
| modified from | |
| LOCUS | NM_000597 1433 bp mRNA linear PRI 31-OCT.-2000 |
| DEFINITION | Homo sapiens insulin-like growth factor binding protein 2 |
| | (36 kD) (IGFBP2), mRNA. |
| ACCESSION | NM_000597 |
| VERSION | NM_000597.1 GI: 10835156 |
| KEYWORDS | • |
| SOURCE | Homo sapiens (human) |
| ORIGIN | |

| [SEQ ID NO: 3] | |
|---|---|
| 1 | attcggggcg agggaggagg aagaagcgga ggaggcggct cccgctcgca gggccgtgca |
| 61 | cctgcccgcc cgcccgctcg ctcgctcgcc cgccgcgccg cgctgccgac cgccagcatg |
| 121 | ctgccgagag tgggctgccc cgcgctgccg ctgccgccgc cgccgctgct gccgctgctg |
| 181 | ccgctgctgc tgctgctact gggcgcgagt ggcggcggcg gcggggcgcg cgcggaggtg |
| 241 | ctgttccgct gcccgccctg cacacccgag cgcctggccg cctgcgggcc cccgccggtt |
| 301 | gcgccgcccg ccgcggtggc cgcagtggcc ggaggcgccc gcatgccatg cgcggagctc |
| 361 | gtccgggagc cgggctgcgg ctgctgctcg gtgtgcgccc ggctggaggg cgaggcgtgc |
| 421 | ggcgtctaca ccccgcgctg cggccagggg ctgcgctgct atccccaccc gggctccgag |
| 481 | ctgcccctgc aggcgctggt catgggcgag ggcacttgtg agaagcgccg ggacgccgag |
| 541 | tatggcgcca gcccggagca ggttgcagac aatggcgatg accactcaga aggaggcctg |
| 601 | gtggagaacc acgtggacag caccatgaac atgttgggcg ggggaggcag tgctggccgg |
| 661 | aagcccctca agtcgggtat gaaggagctg gccgtgttcc gggagaaggt cactgagcag |
| 721 | caccggcaga tgggcaaggg tggcaagcat caccttggcc tggaggagcc caagaagctg |
| 781 | cgaccacccc ctgccaggac tccctgccaa caggaactgg accaggtcct ggagcggatc |
| 841 | tccaccatgc gccttccgga tgagcggggc cctctggagc acctctactc cctgcacatc |
| 901 | cccaactgtg acaagcatgg cctgtacaac ctcaaacagt gcaagatgtc tctgaacggg |
| 961 | cagcgtgggg agtgctggtg tgtgaacccc aacaccggga agctgatcca gggagccccc |
| 1021 | accatccggg gggaacccga gtgtcatctc ttctacaatg agcagcagga ggcttgcggg |
| 1081 | gtgcacaccc agcggatgca gtagaccgca gccagccggt gcctggcgcc cctgccccce |
| 1141 | gcccctctcc aaacaccggc agaaaacgga gagtgcttgg gtggtgggtg ctggaggatt |
| 1201 | ttccagttct gacacacgta tttatatttg gaaagagacc agcaccgagc tcggcacctc |
| 1261 | cccggcctct ctcttcccag ctgcagatgc cacacctgct ccttcttgct ttccccgggg |
| 1321 | gaggaagggg gttgtggtcg gggagctggg gtacaggttt ggggaggggg aagagaaatt |
| 1381 | tttatttttg aacccctgtg tcccttttgc ataagattaa aggaaggaaa agt |

IGFBP-2 [Homo sapiens].

Further coding sequences may be deduced with the following table

1-7

| Met | Leu | Pro | Arg | Val | Gly | Cys |
|---|---|---|---|---|---|---|
| AUG 1.00 | CUG 0.83 | CCG 0.77 | CGU 0.74 | GUU 0.51 | GGU 0.59 | UGC 0.51 |
| | CUC 0.07 | CCA 0.15 | CGC 0.25 | GUA 0.26 | GGC 0.38 | UGU 0.49 |
| | CUU 0.04 | CCU 0.08 | CGA 0.01 | GUG 0.16 | GGG 0.02 | |
| | UUG 0.03 | CCC 0.00 | AGG 0.00 | GUC 0.07 | GGA 0.00 | |
| | UUA 0.02 | | AGA 0.00 | | | |
| | CUA 0.00 | | CGG 0.00 | | | |

8-14

| Pro | Ala | Leu | Pro | Leu | Pro | Pro |
|---|---|---|---|---|---|---|
| CCG 0.77 | GCU 0.35 | CUG 0.83 | CCG 0.77 | CUG 0.83 | CCG 0.77 | CCG 0.77 |
| CCA 0.15 | GCA 0.28 | CUC 0.07 | CCA 0.15 | CUC 0.07 | CCA 0.15 | CCA 0.15 |
| CCU 0.08 | GCG 0.26 | CUU 0.04 | CCU 0.08 | CUU 0.04 | CCU 0.08 | CCU 0.08 |
| CCC 0.00 | GCC 0.10 | UUG 0.03 | CCC 0.00 | UUG 0.03 | CCC 0.00 | CCC 0.00 |
| | | UUA 0.02 | | UUA 0.02 | | |
| | | CUA 0.00 | | CUA 0.00 | | |

15-21

| Pro | Pro | Leu | Leu | Pro | Leu | Leu |
|---|---|---|---|---|---|---|
| CCG 0.77 | CCG 0.77 | CUG 0.83 | CUG 0.83 | CCG 0.77 | CUG 0.83 | CUG 0.83 |
| CCA 0.15 | CCA 0.15 | CUC 0.07 | CUC 0.07 | CCA 0.15 | CUC 0.07 | CUC 0.07 |
| CCU 0.08 | CCU 0.08 | CUU 0.04 | CUU 0.04 | CCU 0.08 | CUU 0.04 | CUU 0.04 |
| CCC 0.00 | CCC 0.00 | UUG 0.03 | UUG 0.03 | CCC 0.00 | UUG 0.03 | UUG 0.03 |
| | | UUA 0.02 | UUA 0.02 | | UUA 0.02 | UUA 0.02 |
| | | CUA 0.00 | CUA 0.00 | | CUA 0.00 | CUA 0.00 |

22-28

| Pro | Leu | Leu | Leu | Leu | Leu | Leu |
|---|---|---|---|---|---|---|
| CCG 0.77 | CUG 0.83 | CUG 0.83 | CUG 0.83 | CUG 0.83 | CUG 0.83 | CUG 0.83 |
| CCA 0.15 | CUC 0.07 | CUC 0.07 | CUC 0.07 | CUC 0.07 | CUC 0.07 | CUC 0.07 |
| CCU 0.08 | CUU 0.04 | CUU 0.04 | CUU 0.04 | CUU 0.04 | CUU 0.04 | CUU 0.04 |
| CCC 0.00 | UUG 0.03 | UUG 0.03 | UUG 0.03 | UUG 0.03 | UUG 0.03 | UUG 0.03 |
| | UUA 0.02 | UUA 0.02 | UUA 0.02 | UUA 0.02 | UUA 0.02 | UUA 0.02 |
| | CUA 0.00 | CUA 0.00 | CUA 0.00 | CUA 0.00 | CUA 0.00 | CUA 0.00 |

29-35

| Gly | Ala | Ser | Gly | Gly | Gly | Gly |
|---|---|---|---|---|---|---|
| GGU 0.59 | GCU 0.35 | UCC 0.37 | GGU 0.59 | GGU 0.59 | GGU 0.59 | GGU 0.59 |
| GGC 0.38 | GCA 0.28 | UCU 0.34 | GGC 0.38 | GGC 0.38 | GGC 0.38 | GGC 0.38 |
| GGG 0.02 | GCG 0.26 | AGC 0.20 | GGG 0.02 | GGG 0.02 | GGG 0.02 | GGG 0.02 |
| GGA 0.00 | GCC 0.10 | UCG 0.04 | GGA 0.00 | GGA 0.00 | GGA 0.00 | GGA 0.00 |
| | | AGU 0.03 | | | | |
| | | UCA 0.02 | | | | |

36-42

| Gly | Ala | Arg | Ala | Glu | Val | Leu |
|---|---|---|---|---|---|---|
| GGU 0.59 | GCU 0.35 | CGU 0.74 | GCU 0.35 | GAA 0.78 | GUU 0.51 | CUG 0.83 |
| GGC 0.38 | GCA 0.28 | CGC 0.25 | GCA 0.28 | GAG 0.22 | GUA 0.26 | CUC 0.07 |
| GGG 0.02 | GCG 0.26 | CGA 0.01 | GCG 0.26 | | GUG 0.16 | CUU 0.04 |
| GGA 0.00 | GCC 0.10 | AGG 0.00 | GCC 0.10 | | GUC 0.07 | UUG 0.03 |
| | | AGA 0.00 | | | | UUA 0.02 |
| | | CGG 0.00 | | | | CUA 0.00 |

43-49

| Phe | Arg | Cys | Pro | Pro | Cys | Thr |
|---|---|---|---|---|---|---|
| UUC 0.76 | CGU 0.74 | UGC 0.51 | CCG 0.77 | CCG 0.77 | UGC 0.51 | ACC 0.55 |
| UUU 0.24 | CGC 0.25 | UGU 0.49 | CCA 0.15 | CCA 0.15 | UGU 0.49 | ACU 0.35 |
| | CGA 0.01 | | CCU 0.08 | CCU 0.08 | | ACG 0.07 |
| | AGG 0.00 | | CCC 0.00 | CCC 0.00 | | ACA 0.04 |
| | AGA 0.00 | | | | | |
| | CGG 0.00 | | | | | |

50-56

| Pro | Glu | Arg | Leu | Ala | Ala | Cys |
|---|---|---|---|---|---|---|
| CCG 0.77 | GAA 0.78 | CGU 0.74 | CUG 0.83 | GCU 0.35 | GCU 0.35 | UGC 0.51 |
| CCA 0.15 | GAG 0.22 | CGC 0.25 | CUC 0.07 | GCA 0.28 | GCA 0.28 | UGU 0.49 |
| CCU 0.08 | | CGA 0.01 | CUU 0.04 | GCG 0.26 | GCG 0.26 | |
| CCC 0.00 | | AGG 0.00 | UUG 0.03 | GCC 0.10 | GCC 0.10 | |
| | | AGA 0.00 | UUA 0.02 | | | |
| | | CGG 0.00 | CUA 0.00 | | | |

57-63

| Gly | Pro | Pro | Pro | Val | Ala | Pro |
|---|---|---|---|---|---|---|
| GGU 0.59 | CCG 0.77 | CCG 0.77 | CCG 0.77 | GUU 0.51 | GCU 0.35 | CCG 0.77 |
| GGC 0.38 | CCA 0.15 | CCA 0.15 | CCA 0.15 | GUA 0.26 | GCA 0.28 | CCA 0.15 |
| GGG 0.02 | CCU 0.08 | CCU 0.08 | CCU 0.08 | GUG 0.16 | GCG 0.26 | CCU 0.08 |
| GGA 0.00 | CCC 0.00 | CCC 0.00 | CCC 0.00 | GUC 0.07 | GCC 0.10 | CCC 0.00 |

64-70

| Pro | Ala | Ala | Val | Ala | Ala | Val |
|---|---|---|---|---|---|---|
| CCG 0.77 | GCU 0.35 | GCU 0.35 | GUU 0.51 | GCU 0.35 | GCU 0.35 | GUU 0.51 |
| CCA 0.15 | GCA 0.28 | GCA 0.28 | GUA 0.26 | GCA 0.28 | GCA 0.28 | GUA 0.26 |
| CCU 0.08 | GCG 0.26 | GCG 0.26 | GUG 0.16 | GCG 0.26 | GCG 0.26 | GUG 0.16 |
| CCC 0.00 | GCC 0.10 | GCC 0.10 | GUC 0.07 | GCC 0.10 | GCC 0.10 | GUC 0.07 |

71-77

| Ala | Gly | Gly | Ala | Arg | Met | Pro |
|---|---|---|---|---|---|---|
| GCU 0.35 | GGU 0.59 | GGU 0.59 | GCU 0.35 | CGU 0.74 | AUG 1.00 | CCG 0.77 |
| GCA 0.28 | GGC 0.38 | GGC 0.38 | GCA 0.28 | CGC 0.25 | | CCA 0.15 |
| GCG 0.26 | GGG 0.02 | GGG 0.02 | GCG 0.26 | CGA 0.01 | | CCU 0.08 |
| GCC 0.10 | GGA 0.00 | GGA 0.00 | GCC 0.10 | AGG 0.00 | | CCC 0.00 |
| | | | | AGA 0.00 | | |
| | | | | CGG 0.00 | | |

78-84

| Cys | Ala | Glu | Leu | Val | Arg | Glu |
|---|---|---|---|---|---|---|
| UGC 0.51 | GCU 0.35 | GAA 0.78 | CUG 0.83 | GUU 0.51 | CGU 0.74 | GAA 0.78 |
| UGU 0.49 | GCA 0.28 | GAG 0.22 | CUC 0.07 | GUA 0.26 | CGC 0.25 | GAG 0.22 |
| | GCG 0.26 | | CUU 0.04 | GUG 0.16 | CGA 0.01 | |
| | GCC 0.10 | | UUG 0.03 | GUC 0.07 | AGG 0.00 | |
| | | | UUA 0.02 | | AGA 0.00 | |
| | | | CUA 0.00 | | CGG 0.00 | |

85-91

| Pro | Gly | Cys | Gly | Cys | Cys | Ser |
|---|---|---|---|---|---|---|
| CCG 0.77 | GGU 0.59 | UGC 0.51 | GGU 0.59 | UGC 0.51 | UGC 0.51 | UCC 0.37 |
| CCA 0.15 | GGC 0.38 | UGU 0.49 | GGC 0.38 | UGU 0.49 | UGU 0.49 | UCU 0.34 |
| CCU 0.08 | GGG 0.02 | | GGG 0.02 | | | AGC 0.20 |
| CCC 0.00 | GGA 0.00 | | GGA 0.00 | | | UCG 0.04 |
| | | | | | | AGU 0.03 |
| | | | | | | UCA 0.02 |

92-98

| Val | Cys | Ala | Arg | Leu | Glu | Gly |
|---|---|---|---|---|---|---|
| GUU 0.51 | UGC 0.51 | GCU 0.35 | CGU 0.74 | CUG 0.83 | GAA 0.78 | GGU 0.59 |
| GUA 0.26 | UGU 0.49 | GCA 0.28 | CGC 0.25 | CUC 0.07 | GAG 0.22 | GGC 0.38 |
| GUG 0.16 | | GCG 0.26 | CGA 0.01 | CUU 0.04 | | GGG 0.02 |
| GUC 0.07 | | GCC 0.10 | AGG 0.00 | UUG 0.03 | | GGA 0.00 |
| | | | AGA 0.00 | UUA 0.02 | | |
| | | | CGG 0.00 | CUA 0.00 | | |

99-105

| Glu | Ala | Cys | Gly | Val | Tyr | Thr |
|---|---|---|---|---|---|---|
| GAA 0.78 | GCU 0.35 | UGC 0.51 | GGU 0.59 | GUU 0.51 | UAC 0.75 | ACC 0.55 |
| GAG 0.22 | GCA 0.28 | UGU 0.49 | GGC 0.38 | GUA 0.26 | UAU 0.25 | ACU 0.35 |
| | GCG 0.26 | | GGG 0.02 | GUG 0.16 | | ACG 0.07 |
| | GCC 0.10 | | GGA 0.00 | GUC 0.07 | | ACA 0.04 |

106-112

| Pro | Arg | Cys | Gly | Gln | Gly | Leu |
|---|---|---|---|---|---|---|
| CCG 0.77 | CGU 0.74 | UGC 0.51 | GGU 0.59 | CAG 0.86 | GGU 0.59 | CUG 0.83 |
| CCA 0.15 | CGC 0.25 | UGU 0.49 | GGC 0.38 | CAA 0.14 | GGC 0.38 | CUC 0.07 |
| CCU 0.08 | CGA 0.01 | | GGG 0.02 | | GGG 0.02 | CUU 0.04 |
| CCC 0.00 | AGG 0.00 | | GGA 0.00 | | GGA 0.00 | UUG 0.03 |
| | AGA 0.00 | | | | | UUA 0.02 |
| | CGG 0.00 | | | | | CUA 0.00 |

113-119

| Arg | Cys | Tyr | Pro | His | Pro | Gly |
|---|---|---|---|---|---|---|
| CGU 0.74 | UGC 0.51 | UAC 0.75 | CCG 0.77 | CAC 0.83 | CCG 0.77 | GGU 0.59 |
| CGC 0.25 | UGU 0.49 | UAU 0.25 | CCA 0.15 | CAU 0.17 | CCA 0.15 | GGC 0.38 |
| CGA 0.01 | | | CCU 0.08 | | CCU 0.08 | GGG 0.02 |
| AGG 0.00 | | | CCC 0.00 | | CCC 0.00 | GGA 0.00 |
| AGA 0.00 | | | | | | |
| CGG 0.00 | | | | | | |

120-126

| Ser | Glu | Leu | Pro | Leu | Gln | Ala |
|---|---|---|---|---|---|---|
| UCC 0.37 | GAA 0.78 | CUG 0.83 | CCG 0.77 | CUG 0.83 | CAG 0.86 | GCU 0.35 |
| UCU 0.34 | GAG 0.22 | CUC 0.07 | CCA 0.15 | CUC 0.07 | CAA 0.14 | GCA 0.28 |
| AGC 0.20 | | CUU 0.04 | CCU 0.08 | CUU 0.04 | | GCG 0.26 |
| UCG 0.04 | | UUG 0.03 | CCC 0.00 | UUG 0.03 | | GCC 0.10 |
| AGU 0.03 | | UUA 0.02 | | UUA 0.02 | | |
| UCA 0.02 | | CUA 0.00 | | CUA 0.00 | | |

127-133

| Leu | Val | Met | Gly | Glu | Gly | Thr |
|---|---|---|---|---|---|---|
| CUG 0.83 | GUU 0.51 | AUG 1.00 | GGU 0.59 | GAA 0.78 | GGU 0.59 | ACC 0.55 |
| CUC 0.07 | GUA 0.26 | | GGC 0.38 | GAG 0.22 | GGC 0.38 | ACU 0.35 |
| CUU 0.04 | GUG 0.16 | | GGG 0.02 | | GGG 0.02 | ACG 0.07 |
| UUG 0.03 | GUC 0.07 | | GGA 0.00 | | GGA 0.00 | ACA 0.04 |
| UUA 0.02 | | | | | | |
| CUA 0.00 | | | | | | |

134-140

| Cys | Glu | Lys | Arg | Arg | Asp | Ala |
|---|---|---|---|---|---|---|
| UGC 0.51 | GAA 0.78 | AAA 0.74 | CGU 0.74 | CGU 0.74 | GAC 0.67 | GCU 0.35 |
| UGU 0.49 | GAG 0.22 | AAG 0.26 | CGC 0.25 | CGC 0.25 | GAU 0.33 | GCA 0.28 |
| | | | CGA 0.01 | CGA 0.01 | | GCG 0.26 |
| | | | AGG 0.00 | AGG 0.00 | | GCC 0.10 |
| | | | AGA 0.00 | AGA 0.00 | | |
| | | | CGG 0.00 | CGG 0.00 | | |
| 218 | 316 | 272 | 128 | 135 | 137 | 121 |

141-147

| Glu | Tyr | Gly | Ala | Ser | Pro | Glu |
|---|---|---|---|---|---|---|
| GAA 0.78 | UAC 0.75 | GGU 0.59 | GCU 0.35 | UCC 0.37 | CCC 0.77 | GAA 0.78 |
| GAG 0.22 | UAU 0.25 | GGC 0.38 | GCA 0.28 | UCU 0.34 | CCA 0.15 | GAG 0.22 |
| | | GGG 0.02 | GCG 0.26 | AGC 0.20 | CCU 0.08 | |
| | | GGA 0.00 | GCC 0.10 | UCG 0.04 | CCC 0.00 | |
| | | | | AGU 0.03 | | |
| | | | | UCA 0.02 | | |

148-154

| Gln | Val | Ala | Asp | Asn | Gly | Asp |
|---|---|---|---|---|---|---|
| CAG 0.86 | GUU 0.51 | GCU 0.35 | GAC 0.67 | AAC 0.94 | GGU 0.59 | GAC 0.67 |
| CAA 0.14 | GUA 0.26 | GCA 0.28 | GAU 0.33 | AAU 0.06 | GGC 0.38 | GAU 0.33 |
| | GUG 0.16 | GCG 0.26 | | | GGG 0.02 | |
| | GUC 0.07 | GCC 0.10 | | | GGA 0.00 | |

155-161

| Asp | His | Ser | Glu | Gly | Gly | Leu |
|---|---|---|---|---|---|---|
| GAC 0.67 | CAC 0.83 | UCC 0.37 | GAA 0.78 | GGU 0.59 | GGU 0.59 | CUG 0.83 |
| GAU 0.33 | CAU 0.17 | UCU 0.34 | GAG 0.22 | GGC 0.38 | GGC 0.38 | CUC 0.07 |
| | | AGC 0.20 | | GGG 0.02 | GGG 0.02 | CUU 0.04 |
| | | UCG 0.04 | | GGA 0.00 | GGA 0.00 | UUG 0.03 |
| | | AGU 0.03 | | | | UUA 0.02 |
| | | UCA 0.02 | | | | CUA 0.00 |

162-168

| Val | Glu | Asn | His | Val | Asp | Ser |
|---|---|---|---|---|---|---|
| GUU 0.51 | GAA 0.78 | AAC 0.94 | CAC 0.83 | GUU 0.51 | GAC 0.67 | UCC 0.37 |
| GUA 0.26 | GAG 0.22 | AAU 0.06 | CAU 0.17 | GUA 0.26 | GAU 0.33 | UCU 0.34 |
| GUG 0.16 | | | | GUG 0.16 | | AGC 0.20 |
| GUC 0.07 | | | | GUC 0.07 | | UCG 0.04 |
| | | | | | | AGU 0.03 |
| | | | | | | UCA 0.02 |

169-175

| Thr | Met | Asn | Met | Leu | Gly | Gly |
|---|---|---|---|---|---|---|
| ACC 0.55 | AUG 1.00 | AAC 0.94 | AUG 1.00 | CUG 0.83 | GGU 0.59 | GGU 0.59 |
| ACU 0.35 | | AAU 0.06 | | CUC 0.07 | GGC 0.38 | GGC 0.38 |
| ACG 0.07 | | | | CUU 0.04 | GGG 0.02 | GGG 0.02 |
| ACA 0.04 | | | | UUG 0.03 | GGA 0.00 | GGA 0.00 |
| | | | | UUA 0.02 | | |
| | | | | CUA 0.00 | | |

176-182

| Gly | Gly | Ser | Ala | Gly | Arg | Lys |
|---|---|---|---|---|---|---|
| GGU 0.59 | GGU 0.59 | UCC 0.37 | GCU 0.35 | GGU 0.59 | CGU 0.74 | AAA 0.74 |
| GGC 0.38 | GGC 0.38 | UCU 0.34 | GCA 0.28 | GGC 0.38 | CGC 0.25 | AAG 0.26 |
| GGG 0.02 | GGG 0.02 | AGC 0.20 | GCG 0.26 | GGG 0.02 | CGA 0.01 | |
| GGA 0.00 | GGA 0.00 | UCG 0.04 | GCC 0.10 | GGA 0.00 | AGG 0.00 | |
| | | AGU 0.03 | | | AGA 0.00 | |
| | | UCA 0.02 | | | CGG 0.00 | |

183-189

| Pro | Leu | Lys | Ser | Gly | Met | Lys |
|---|---|---|---|---|---|---|
| CCC 0.77 | CUG 0.83 | AAA 0.74 | UCC 0.37 | GCU 0.59 | AUG 1.00 | AAA 0.74 |
| CCA 0.15 | CUC 0.07 | AAG 0.26 | UCU 0.34 | GGC 0.38 | | AAG 0.26 |
| CCU 0.08 | CUU 0.04 | | AGC 0.20 | GGG 0.02 | | |
| CCC 0.00 | UUG 0.03 | | UCG 0.04 | GGA 0.00 | | |
| | UUA 0.02 | | AGU 0.03 | | | |
| | CUA 0.00 | | UCA 0.02 | | | |

190-196

| Glu | Leu | Ala | Val | Phe | Arg | Glu |
|---|---|---|---|---|---|---|
| GAA 0.78 | CUG 0.83 | GCU 0.35 | GUU 0.51 | UUC 0.76 | CGU 0.74 | GAA 0.78 |
| GAG 0.22 | CUC 0.07 | GCA 0.28 | GUA 0.26 | UUU 0.24 | CGC 0.25 | GAG 0.22 |
| | CUU 0.04 | GCG 0.26 | GUG 0.16 | | CGA 0.01 | |
| | UUG 0.03 | GCC 0.10 | GUC 0.07 | | AGG 0.00 | |
| | UUA 0.02 | | | | AGA 0.00 | |
| | CUA 0.00 | | | | CGG 0.00 | |

197-203

| Lys | Val | Thr | Glu | Gln | His | Arg |
|---|---|---|---|---|---|---|
| AAA 0.74 | GUU 0.51 | ACC 0.55 | GAA 0.78 | CAG 0.86 | CAC 0.83 | CGU 0.74 |
| AAG 0.26 | GUA 0.26 | ACU 0.35 | GAG 0.22 | CAA 0.14 | CAU 0.17 | CGC 0.25 |
| | GUG 0.16 | ACG 0.07 | | | | CGA 0.01 |
| | GUC 0.07 | ACA 0.04 | | | | AGG 0.00 |
| | | | | | | AGA 0.00 |
| | | | | | | CGG 0.00 |

204-210

| Gln | Met | Gly | Lys | Gly | Gly | Lys |
|---|---|---|---|---|---|---|
| CAG 0.86 | AUG 1.00 | GGU 0.59 | AAA 0.74 | GGU 0.59 | GGU 0.59 | AAA 0.74 |
| CAA 0.14 | | GGC 0.38 | AAG 0.26 | GGC 0.38 | GGC 0.38 | AAG 0.26 |
| | | GGG 0.02 | | GGG 0.02 | GGG 0.02 | |
| | | GGA 0.00 | | GGA 0.00 | GGA 0.00 | |

211-217

| His | His | Leu | Gly | Leu | Glu | Glu |
|---|---|---|---|---|---|---|
| CAC 0.83 | CAC 0.83 | CUG 0.83 | GGU 0.59 | CUG 0.83 | GAA 0.78 | GAA 0.78 |
| CAU 0.17 | CAU 0.17 | CUC 0.07 | GGC 0.38 | CUC 0.07 | GAG 0.22 | GAG 0.22 |
| | | CUU 0.04 | GGG 0.02 | CUU 0.04 | | |
| | | UUG 0.03 | GGA 0.00 | UUG 0.03 | | |
| | | UUA 0.02 | | UUA 0.02 | | |
| | | CUA 0.00 | | CUA 0.00 | | |

218-224

| Pro | Lys | Lys | Leu | Arg | Pro | Pro |
|---|---|---|---|---|---|---|
| CCG 0.77 | AAA 0.74 | AAA 0.74 | CUG 0.83 | CGU 0.74 | CCG 0.77 | CCG 0.77 |
| CCA 0.15 | AAG 0.26 | AAG 0.26 | CUC 0.07 | CGC 0.25 | CCA 0.15 | CCA 0.15 |
| CCU 0.08 | | | CUU 0.04 | CGA 0.01 | CCU 0.08 | CCU 0.08 |
| CCC 0.00 | | | UUG 0.03 | AGG 0.00 | CCC 0.00 | CCC 0.00 |
| | | | UUA 0.02 | AGA 0.00 | | |
| | | | CUA 0.00 | CGG 0.00 | | |

225-231

| Pro | Ala | Arg | Thr | Pro | Cys | Gln |
|---|---|---|---|---|---|---|
| CCG 0.77 | GCU 0.35 | CGU 0.74 | ACC 0.55 | CCG 0.77 | UGC 0.51 | CAG 0.86 |
| CCA 0.15 | GCA 0.28 | CGC 0.25 | ACU 0.35 | CCA 0.15 | UGU 0.49 | CAA 0.14 |
| CCU 0.08 | GCG 0.26 | CGA 0.01 | ACG 0.07 | CCU 0.08 | | |
| CCC 0.00 | GCC 0.10 | AGG 0.00 | ACA 0.04 | CCC 0.00 | | |
| | | AGA 0.00 | | | | |
| | | CGG 0.00 | | | | |

232-238

| Gln | Glu | Leu | Asp | Gln | Val | Leu |
|---|---|---|---|---|---|---|
| CAG 0.86 | GAA 0.78 | CUG 0.83 | GAC 0.67 | CAG 0.86 | GUU 0.51 | CUG 0.83 |
| CAA 0.14 | GAG 0.22 | CUC 0.07 | GAU 0.33 | CAA 0.14 | GUA 0.26 | CUC 0.07 |
| | | CUU 0.04 | | | GUG 0.16 | CUU 0.04 |
| | | UUG 0.03 | | | GUC 0.07 | UUG 0.03 |
| | | UUA 0.02 | | | | UUA 0.02 |
| | | CUA 0.00 | | | | CUA 0.00 |

239-245

| Glu | Arg | Ile | Ser | Thr | Met | Arg |
|---|---|---|---|---|---|---|
| GAA 0.78 | CGU 0.74 | AUC 0.83 | UCC 0.37 | ACC 0.55 | AUG 1.00 | CGU 0.74 |
| GAG 0.22 | CGC 0.25 | AUU 0.17 | UCU 0.34 | ACU 0.35 | | CGC 0.25 |
| | CGA 0.01 | AUA 0.00 | AGC 0.20 | ACG 0.07 | | CGA 0.01 |
| | AGG 0.00 | | UCG 0.04 | ACA 0.04 | | AGG 0.00 |
| | AGA 0.00 | | AGU 0.03 | | | AGA 0.00 |
| | CGG 0.00 | | UCA 0.02 | | | CGG 0.00 |

246-252

| Leu | Pro | Asp | Glu | Arg | Gly | Pro |
|---|---|---|---|---|---|---|
| CUG 0.83 | CCG 0.77 | GAC 0.67 | GAA 0.78 | CGU 0.74 | GGU 0.59 | CCG 0.77 |
| CUC 0.07 | CCA 0.15 | GAU 0.33 | GAG 0.22 | CGC 0.25 | GGC 0.38 | CCA 0.15 |
| CUU 0.04 | CCU 0.08 | | | CGA 0.01 | GGG 0.02 | CCU 0.08 |
| UUG 0.03 | CCC 0.00 | | | AGG 0.00 | GGA 0.00 | CCC 0.00 |
| UUA 0.02 | | | | AGA 0.00 | | |
| CUA 0.00 | | | | CGG 0.00 | | |

253-259

| Leu | Glu | His | Leu | Tyr | Ser | Leu |
|---|---|---|---|---|---|---|
| CUG 0.83 | GAA 0.78 | CAC 0.83 | CUG 0.83 | UAC 0.75 | UCC 0.37 | CUG 0.83 |
| CUC 0.07 | GAG 0.22 | CAU 0.17 | CUC 0.07 | UAU 0.25 | UCU 0.34 | CUC 0.07 |
| CUU 0.04 | | | CUU 0.04 | | AGC 0.20 | CUU 0.04 |
| UUG 0.03 | | | UUG 0.03 | | UCG 0.04 | UUG 0.03 |
| UUA 0.02 | | | UUA 0.02 | | AGU 0.03 | UUA 0.02 |
| CUA 0.00 | | | CUA 0.00 | | UCA 0.02 | CUA 0.00 |

260-266

| His | Ile | Pro | Asn | Cys | Asp | Lys |
|---|---|---|---|---|---|---|
| CAC 0.83 | AUC 0.83 | CCG 0.77 | AAC 0.94 | UGC 0.51 | GAC 0.67 | AAA 0.74 |
| CAU 0.17 | AUU 0.17 | CCA 0.15 | AAU 0.06 | UGU 0.49 | GAU 0.33 | AAG 0.26 |
| | AUA 0.00 | CCU 0.08 | | | | |
| | | CCC 0.00 | | | | |

267-273

| His | Gly | Leu | Tyr | Asn | Leu | Lys |
|---|---|---|---|---|---|---|
| CAC 0.83 | GGU 0.59 | CUG 0.83 | UAC 0.75 | AAC 0.94 | CUG 0.83 | AAA 0.74 |
| CAU 0.17 | GGC 0.38 | CUC 0.07 | UAU 0.25 | AAU 0.06 | CUC 0.07 | AAG 0.26 |
| | GGG 0.02 | CUU 0.04 | | | CUU 0.04 | |
| | GGA 0.00 | UUG 0.03 | | | UUG 0.03 | |
| | | UUA 0.02 | | | UUA 0.02 | |
| | | CUA 0.00 | | | CUA 0.00 | |

274-280

| Gln | Cys | Lys | Met | Ser | Leu | Asn |
|---|---|---|---|---|---|---|
| CAG 0.86 | UGC 0.51 | AAA 0.74 | AUG 1.00 | UCC 0.37 | CUG 0.83 | AAC 0.94 |
| CAA 0.14 | UGU 0.49 | AAG 0.26 | | UCU 0.34 | CUC 0.07 | AAU 0.06 |
| | | | | AGC 0.20 | CUU 0.04 | |
| | | | | UCG 0.04 | UUG 0.03 | |
| | | | | AGU 0.03 | UUA 0.02 | |
| | | | | UCA 0.02 | CUA 0.00 | |

281-287

| Gly | Gln | Arg | Gly | Glu | Cys | Trp |
|---|---|---|---|---|---|---|
| GGU 0.59 | CAG 0.86 | CGU 0.74 | GGU 0.59 | GAA 0.78 | UGC 0.51 | UGG 1.00 |
| GGC 0.38 | CAA 0.14 | CGC 0.25 | GGC 0.38 | GAG 0.22 | UGU 0.49 | |
| GGG 0.02 | | CGA 0.01 | GGG 0.02 | | | |
| GGA 0.00 | | AGG 0.00 | GGA 0.00 | | | |
| | | AGA 0.00 | | | | |
| | | CGG 0.00 | | | | |

288-294

| Cys | Val | Asn | Pro | Asn | Thr | Gly |
|---|---|---|---|---|---|---|
| UGC 0.51 | GUU 0.51 | AAC 0.94 | CCG 0.77 | AAC 0.94 | ACC 0.55 | GGU 0.59 |
| UGU 0.49 | GUA 0.26 | AAU 0.06 | CCA 0.15 | AAU 0.06 | ACU 0.35 | GGC 0.38 |
| | GUG 0.16 | | CCU 0.08 | | ACG 0.07 | GGG 0.02 |
| | GUC 0.07 | | CCC 0.00 | | ACA 0.04 | GGA 0.00 |

295-301

| Lys | Leu | Ile | Gln | Gly | Ala | Pro |
|---|---|---|---|---|---|---|
| AAA 0.74 | CUG 0.83 | AUC 0.83 | CAG 0.86 | GGU 0.59 | GCU 0.35 | CCG 0.77 |
| AAG 0.26 | CUC 0.07 | AUU 0.17 | CAA 0.14 | GGC 0.38 | GCA 0.28 | CCA 0.15 |
| | CUU 0.04 | AUA 0.00 | | GGG 0.02 | GCG 0.26 | CCU 0.08 |
| | UUG 0.03 | | | GGA 0.00 | GCC 0.10 | CCC 0.00 |
| | UUA 0.02 | | | | | |
| | CUA 0.00 | | | | | |

302-308

| Thr | Ile | Arg | Gly | Glu | Pro | Glu |
|---|---|---|---|---|---|---|
| ACC 0.55 | AUC 0.83 | CGU 0.74 | GGU 0.59 | GAA 0.67 | CCG 0.77 | GAA 0.78 |
| ACU 0.35 | AUU 0.17 | CGC 0.25 | GGC 0.38 | GAG 0.33 | CCA 0.15 | GAG 0.22 |
| ACG 0.07 | AUA 0.00 | CGA 0.01 | GGG 0.02 | | CCU 0.08 | |
| ACA 0.04 | | AGG 0.00 | GGA 0.00 | | CCC 0.00 | |
| | | AGA 0.00 | | | | |
| | | CGG 0.00 | | | | |

309-315

| Cys | His | Leu | Phe | Tyr | Asn | Glu |
|---|---|---|---|---|---|---|
| UGC 0.51 | CAC 0.83 | CUG 0.83 | UUC 0.76 | UAC 0.75 | AAC 0.94 | GAA 0.78 |
| UGU 0.49 | CAU 0.17 | CUC 0.07 | UUU 0.24 | UAU 0.25 | AAU 0.06 | GAG 0.22 |
| | | CUU 0.04 | | | | |
| | | UUG 0.03 | | | | |
| | | UUA 0.02 | | | | |
| | | CUA 0.00 | | | | |

316-322

| Gln | Gln | Glu | Ala | Cys | Gly | Val |
|---|---|---|---|---|---|---|
| CAG 0.86 | CAG 0.86 | GAA 0.78 | GCU 0.35 | UGC 0.51 | GGU 0.59 | GUU 0.51 |
| CAA 0.14 | CAA 0.14 | GAG 0.22 | GCA 0.28 | UGU 0.49 | GGC 0.38 | GUA 0.26 |
| | | | GCG 0.26 | | GGG 0.02 | GUG 0.16 |
| | | | GCC 0.10 | | GGA 0.00 | GUC 0.07 |

323-328

| His | Thr | Gln | Arg | Met | Gln |
|---|---|---|---|---|---|
| CAC 0.83 | ACC 0.55 | CAG 0.86 | CGU 0.74 | AUG 1.00 | CAG 0.86 |
| CAU 0.17 | ACU 0.35 | CAA 0.14 | CGC 0.25 | | CAA 0.14 |
| | ACG 0.07 | | CGA 0.01 | | |
| | ACA 0.04 | | AGG 0.00 | | |
| | | | AGA 0.00 | | |
| | | | CGG 0.00 | | |

SEQ ID NO: 4

| RGE-IGFBP-2 [Homo sapiens]. | |
|---|---|
| Modified from ACCESSION NP_000588 | |
| 1 | mlprvgcpal plppppllpl lpllllllga sgggggarae vlfrcppctp |
| 51 | erlaacgppp vappaavaav aggarmpcae lvrepgcgcc svcarlegea |
| 101 | cgvytprcgq glrcyphpgs elplqalvmg egtcekrrda eygaspeqva |
| 151 | dngddhsegg lvenhvdstm nmlggggsag rkplksgmke lavfrekvte |
| 201 | qhrqmgkggk hhlgleepkk lrpppartpc qqeldqvler istmrlpder |
| 251 | gplehlyslh ipncdkhgly nlkqckmsln gqrgecwcvn pntgkliqga |
| 301 | ptirgepech lfyneqqeac gvhtqrmq |

| SEQ ID NO: 5 | |
|---|---|
| PQQLNP-IGFBP-2 modifiziert | |
| LOCUS | NM_000597 1433 bp mRNA linear PRI 31-OCT.-2000 |
| DEFINITION | Homo sapiens insulin-like growth factor binding protein 2 |
| | (36 kD) (IGFBP2), mRNA. |
| ACCESSION | NM_000597 |
| VERSION | NM_000597.1 GI: 10835156 |
| KEYWORDS | • |
| SOURCE | Homo sapiens (human) |
| ORIGIN | |

| [SEQ ID NO: 5] | |
|---|---|
| 1 | attcggggcg agggaggagg aagaagcgga ggaggcggct cccgctcgca gggccgtgca |
| 61 | cctgcccgcc cgcccgctcg ctcgctcgcc cgccgcgccg cgctgccgac cgccagcatg |
| 121 | ctgccgagag tgggctgccc cgcgctgccg ctgccgccgc cgccgctgct gccgctgctg |
| 181 | ccgctgctgc tgctgctact gggcgcgagt ggcggcggcg gcggggcgcg cgcggaggtg |
| 241 | ctgttccgct gcccgccctg cacaccagag cgcctggccg cctgcgggcc cccgccggtt |
| 301 | gcgccgcccg ccgcggtggc cgcagtggcc ggaggcgccc gcatgccatg cgcggagctc |
| 361 | gtccgggagc cgggctgcgg ctgctgctcg gtgtgcgccc ggctggaggg cgaggcgtgc |
| 421 | ggcgtctaca ccccgcgctg cggccagggg ctgcgctgct atccccaccc gggctccgag |
| 481 | ctgcccctgc aggcgctggt catgggcgag ggcacttgtg agaagcgccg ggacgccgag |
| 541 | tatggcgcca gcccggagca ggttgcagac aatggcgatg accactcaga aggaggcctg |
| 601 | gtggagaacc acgtggacag caccatgaac atgttgggcg ggggaggcag tgctggccgg |
| 661 | aagcccctca agtcgggtat gaaggagctg gccgtgttcc gggagaaggt cactgagcag |
| 721 | caccggcaga tgggcaaggg tggcaagcat caccttggcc tggaggagcc ccagcagctg |
| 781 | aacccacccc ctgccaggac tacctgccaa caggaactgg accaggtcct ggagcggatc |
| 841 | tccaccatgc gccttccgga tgagcggggc cctctggagc acctctactc cctgcacatc |
| 901 | cccaactgtg acaagcatgg cctgtacaac ctcaaacagt gcaagatgtc tctgaacggg |
| 961 | cagcgtgggg agtgctggig tgtgaacccc aacaccggga agctgatcca gggagccccc |
| 1021 | accatccggg gggaccccga gtgtcatctc ttctacaatg agcagcagga ggcttgcggg |
| 1081 | gtgcacaccc agcggatgca gtagaccgca gccagccggt gcctggcgcc cctgcccccc |
| 1141 | gcccctctcc aaacaccggc agaaaacgga gagtgcttgg gtggtgggtg ctggaggatt |
| 1201 | ttccagttct gacacacgta tttatatttg gaaagagacc agcaccgagc tcggcacctc |
| 1261 | cccggcctct ctcttcccag ctgcagatgc cacacctgct ccttcttgct ttccccgggg |
| 1321 | gaggaagggg gttgtggtcg gggagctggg gtacaggttt ggggaggggg aagagaaatt |
| 1381 | tttatttttg aacccctgtg tcccttttgc ataagattaa aggaaggaaa agt |

IGFBP-2 [Homo sapiens].

Further coding sequences may be deduced with the following table

1-7

| Met | Leu | Pro | Arg | Val | Gly | Cys |
|---|---|---|---|---|---|---|
| AUG 1.00 | CUG 0.83 | CCG 0.77 | CGU 0.74 | GUU 0.51 | GGU 0.59 | UGC 0.51 |
| | CUC 0.07 | CCA 0.15 | CGC 0.25 | GUA 0.26 | GGC 0.38 | UGU 0.49 |
| | CUU 0.04 | CCU 0.08 | CGA 0.01 | GUG 0.16 | GGG 0.02 | |
| | UUG 0.03 | CCC 0.00 | AGG 0.00 | GUC 0.07 | GGA 0.00 | |
| | UUA 0.02 | | AGA 0.00 | | | |
| | CUA 0.00 | | CGG 0.00 | | | |

8-14

| Pro | Ala | Leu | Pro | Leu | Pro | Pro |
|---|---|---|---|---|---|---|
| CCG 0.77 | GCU 0.35 | CUG 0.83 | CCG 0.77 | CUG 0.83 | CCG 0.77 | CCG 0.77 |
| CCA 0.15 | GCA 0.28 | CUC 0.07 | CCA 0.15 | CUC 0.07 | CCA 0.15 | CCA 0.15 |
| CCU 0.08 | GCG 0.26 | CUU 0.04 | CCU 0.08 | CUU 0.04 | CCU 0.08 | CCU 0.08 |
| CCC 0.00 | GCC 0.10 | UUG 0.03 | CCC 0.00 | UUG 0.03 | CCC 0.00 | CCC 0.00 |
| | | UUA 0.02 | | UUA 0.02 | | |
| | | CUA 0.00 | | CUA 0.00 | | |

15-21

| Pro | Pro | Leu | Leu | Pro | Leu | Leu |
|---|---|---|---|---|---|---|
| CCG 0.77 | CCG 0.77 | CUG 0.83 | CUG 0.83 | CCG 0.77 | CUG 0.83 | CUG 0.83 |
| CCA 0.15 | CCA 0.15 | CUC 0.07 | CUC 0.07 | CCA 0.15 | CUC 0.07 | CUC 0.07 |
| CCU 0.08 | CCU 0.08 | CUU 0.04 | CUU 0.04 | CCU 0.08 | CUU 0.04 | CUU 0.04 |
| CCC 0.00 | CCC 0.00 | UUG 0.03 | UUG 0.03 | CCC 0.00 | UUG 0.03 | UUG 0.03 |
| | | UUA 0.02 | UUA 0.02 | | UUA 0.02 | UUA 0.02 |
| | | CUA 0.00 | CUA 0.00 | | CUA 0.00 | CUA 0.00 |

22-28

| Pro | Leu | Leu | Leu | Leu | Leu | Leu |
|---|---|---|---|---|---|---|
| CCG 0.77 | CUG 0.83 | CUG 0.83 | CUG 0.83 | CUG 0.83 | CUG 0.83 | CUG 0.83 |
| CCA 0.15 | CUC 0.07 | CUC 0.07 | CUC 0.07 | CUC 0.07 | CUC 0.07 | CUC 0.07 |
| CCU 0.08 | CUU 0.04 | CUU 0.04 | CUU 0.04 | CUU 0.04 | CUU 0.04 | CUU 0.04 |
| CCC 0.00 | UUG 0.03 | UUG 0.03 | UUG 0.03 | UUG 0.03 | UUG 0.03 | UUG 0.03 |
| | UUA 0.02 | UUA 0.02 | UUA 0.02 | UUA 0.02 | UUA 0.02 | UUA 0.02 |
| | CUA 0.00 | CUA 0.00 | CUA 0.00 | CUA 0.00 | CUA 0.00 | CUA 0.00 |

29-35

| Gly | Ala | Ser | Gly | Gly | Gly | Gly |
|---|---|---|---|---|---|---|
| GGU 0.59 | GCU 0.35 | UCC 0.37 | GGU 0.59 | GGU 0.59 | GGU 0.59 | GGU 0.59 |
| GGC 0.38 | GCA 0.28 | UCU 0.34 | GGC 0.38 | GGC 0.38 | GGC 0.38 | GGC 0.38 |
| GGG 0.02 | GCG 0.26 | AGC 0.20 | GGG 0.02 | GGG 0.02 | GGG 0.02 | GGG 0.02 |
| GGA 0.00 | GCC 0.10 | UCG 0.04 | GGA 0.00 | GGA 0.00 | GGA 0.00 | GGA 0.00 |
| | | AGU 0.03 | | | | |
| | | UCA 0.02 | | | | |

36-42

| Gly | Ala | Arg | Ala | Glu | Val | Leu |
|---|---|---|---|---|---|---|
| GGU 0.59 | GCU 0.35 | CGU 0.74 | GCU 0.35 | GAA 0.78 | GUU 0.51 | CUG 0.83 |
| GGC 0.38 | GCA 0.28 | CGC 0.25 | GCA 0.28 | GAG 0.22 | GUA 0.26 | CUC 0.07 |
| GGG 0.02 | GCG 0.26 | CGA 0.01 | GCG 0.26 | | GUG 0.16 | CUU 0.04 |
| GGA 0.00 | GCC 0.10 | AGG 0.00 | GCC 0.10 | | GUC 0.07 | UUG 0.03 |
| | | AGA 0.00 | | | | UUA 0.02 |
| | | CGG 0.00 | | | | CUA 0.00 |

43-49

| Phe | Arg | Cys | Pro | Pro | Cys | Thr |
|---|---|---|---|---|---|---|
| UUC 0.76 | CGU 0.74 | UGC 0.51 | CCG 0.77 | CCG 0.77 | UGC 0.51 | ACC 0.55 |
| UUU 0.24 | CGC 0.25 | UGU 0.49 | CCA 0.15 | CCA 0.15 | UGU 0.49 | ACU 0.35 |
| | CGA 0.01 | | CCU 0.08 | CCU 0.08 | | ACG 0.07 |
| | AGG 0.00 | | CCC 0.00 | CCC 0.00 | | ACA 0.04 |
| | AGA 0.00 | | | | | |
| | CGG 0.00 | | | | | |

50-56

| Pro | Glu | Arg | Leu | Ala | Ala | Cys |
|---|---|---|---|---|---|---|
| CCG 0.77 | GAA 0.78 | CGU 0.74 | CUG 0.83 | GCU 0.35 | GCU 0.35 | UGC 0.51 |
| CCA 0.15 | GAG 0.22 | CGC 0.25 | CUC 0.07 | GCA 0.28 | GCA 0.28 | UGU 0.49 |
| CCU 0.08 | | CGA 0.01 | CUU 0.04 | GCG 0.26 | GCG 0.26 | |
| CCC 0.00 | | AGG 0.00 | UUG 0.03 | GCC 0.10 | GCC 0.10 | |
| | | AGA 0.00 | UUA 0.02 | | | |
| | | CGG 0.00 | CUA 0.00 | | | |

57-63

| Gly | Pro | Pro | Pro | Val | Ala | Pro |
|---|---|---|---|---|---|---|
| GGU 0.59 | CCG 0.77 | CCG 0.77 | CCG 0.77 | GUU 0.51 | GCU 0.35 | CCG 0.77 |
| GGC 0.38 | CCA 0.15 | CCA 0.15 | CCA 0.15 | GUA 0.26 | GCA 0.28 | CCA 0.15 |
| GGG 0.02 | CCU 0.08 | CCU 0.08 | CCU 0.08 | GUG 0.16 | GCG 0.26 | CCU 0.08 |
| GGA 0.00 | CCC 0.00 | CCC 0.00 | CCC 0.00 | GUC 0.07 | GCC 0.10 | CCC 0.00 |

64-70

| Pro | Ala | Ala | Val | Ala | Ala | Val |
|---|---|---|---|---|---|---|
| CCG 0.77 | GCU 0.35 | GCU 0.35 | GUU 0.51 | GCU 0.35 | GCU 0.35 | GUU 0.51 |
| CCA 0.15 | GCA 0.28 | GCA 0.28 | GUA 0.26 | GCA 0.28 | GCA 0.28 | GUA 0.26 |
| CCU 0.08 | GCG 0.26 | GCG 0.26 | GUG 0.16 | GCG 0.26 | GCG 0.26 | GUG 0.16 |
| CCC 0.00 | GCC 0.10 | GCC 0.10 | GUC 0.07 | GCC 0.10 | GCC 0.10 | GUC 0.07 |

71-77

| Ala | Gly | Gly | Ala | Arg | Met | Pro |
|---|---|---|---|---|---|---|
| GCU 0.35 | GGU 0.59 | GGU 0.59 | GCU 0.35 | CGU 0.74 | AUG 1.00 | CCG 0.77 |
| GCA 0.28 | GGC 0.38 | GGC 0.38 | GCA 0.28 | CGC 0.25 | | CCA 0.15 |
| GCG 0.26 | GGG 0.02 | GGG 0.02 | GCG 0.26 | CGA 0.01 | | CCU 0.08 |
| GCC 0.10 | GGA 0.00 | GGA 0.00 | GCC 0.10 | AGG 0.00 | | CCC 0.00 |
| | | | | AGA 0.00 | | |
| | | | | CGG 0.00 | | |

78-84

| Cys | Ala | Glu | Leu | Val | Arg | Glu |
|---|---|---|---|---|---|---|
| UGC 0.51 | GCU 0.35 | GAA 0.78 | CUG 0.83 | GUU 0.51 | CGU 0.74 | GAA 0.78 |
| UGU 0.49 | GCA 0.28 | GAG 0.22 | CUC 0.07 | GUA 0.26 | CGC 0.25 | GAG 0.22 |
| | GCG 0.26 | | CUU 0.04 | GUG 0.16 | CGA 0.01 | |
| | GCC 0.10 | | UUG 0.03 | GUC 0.07 | AGG 0.00 | |
| | | | UUA 0.02 | | AGA 0.00 | |
| | | | CUA 0.00 | | CGG 0.00 | |

85-91

| Pro | Gly | Cys | Gly | Cys | Cys | Ser |
|---|---|---|---|---|---|---|
| CCG 0.77 | GGU 0.59 | UGC 0.51 | GGU 0.59 | UGC 0.51 | UGC 0.51 | UCC 0.37 |
| CCA 0.15 | GGC 0.38 | UGU 0.49 | GGC 0.38 | UGU 0.49 | UGU 0.49 | UCU 0.34 |
| CCU 0.08 | GGG 0.02 | | GGG 0.02 | | | AGC 0.20 |
| CCC 0.00 | GGA 0.00 | | GGA 0.00 | | | UCG 0.04 |
| | | | | | | AGU 0.03 |
| | | | | | | UCA 0.02 |

92-98

| Val | Cys | Ala | Arg | Leu | Glu | Gly |
|---|---|---|---|---|---|---|
| GUU 0.51 | UGC 0.51 | GCU 0.35 | CGU 0.74 | CUG 0.83 | GAA 0.78 | GGU 0.59 |
| GUA 0.26 | UGU 0.49 | GCA 0.28 | CGC 0.25 | CUC 0.07 | GAG 0.22 | GGC 0.38 |
| GUG 0.16 | | GCG 0.26 | CGA 0.01 | CUU 0.04 | | GGG 0.02 |
| GUC 0.07 | | GCC 0.10 | AGG 0.00 | UUG 0.03 | | GGA 0.00 |
| | | | AGA 0.00 | UUA 0.02 | | |
| | | | CGG 0.00 | CUA 0.00 | | |

99-105

| Glu | Ala | Cys | Gly | Val | Tyr | Thr |
|---|---|---|---|---|---|---|
| GAA 0.78 | GCU 0.35 | UGC 0.51 | GGU 0.59 | GUU 0.51 | UAC 0.75 | ACC 0.55 |
| GAG 0.22 | GCA 0.28 | UGU 0.49 | GGC 0.38 | GUA 0.26 | UAU 0.25 | ACU 0.35 |
| | GCG 0.26 | | GGG 0.02 | GUG 0.16 | | ACG 0.07 |
| | GCC 0.10 | | GGA 0.00 | GUC 0.07 | | ACA 0.04 |

106-112

| Pro | Arg | Cys | Gly | Gln | Gly | Leu |
|---|---|---|---|---|---|---|
| CCG 0.77 | CGU 0.74 | UGC 0.51 | GGU 0.59 | CAG 0.86 | GGU 0.59 | CUG 0.83 |
| CCA 0.15 | CGC 0.25 | UGU 0.49 | GGC 0.38 | CAA 0.14 | GGC 0.38 | CUC 0.07 |
| CCU 0.08 | CGA 0.01 | | GGG 0.02 | | GGG 0.02 | CUU 0.04 |
| CCC 0.00 | AGG 0.00 | | GGA 0.00 | | GGA 0.00 | UUG 0.03 |
| | AGA 0.00 | | | | | UUA 0.02 |
| | CGG 0.00 | | | | | CUA 0.00 |

113-119

| Arg | Cys | Tyr | Pro | His | Pro | Gly |
|---|---|---|---|---|---|---|
| CGU 0.74 | UGC 0.51 | UAC 0.75 | CCG 0.77 | CAC 0.83 | CCG 0.77 | GGU 0.59 |
| CGC 0.25 | UGU 0.49 | UAU 0.25 | CCA 0.15 | CAU 0.17 | CCA 0.15 | GGC 0.38 |
| CGA 0.01 | | | CCU 0.08 | | CCU 0.08 | GGG 0.02 |
| AGG 0.00 | | | CCC 0.00 | | CCC 0.00 | GGA 0.00 |
| AGA 0.00 | | | | | | |
| CGG 0.00 | | | | | | |

120-126

| Ser | Glu | Leu | Pro | Leu | Gln | Ala |
|---|---|---|---|---|---|---|
| UCC 0.37 | GAA 0.78 | CUG 0.83 | CCG 0.77 | CUG 0.83 | CAG 0.86 | GCU 0.35 |
| UCU 0.34 | GAG 0.22 | CUC 0.07 | CCA 0.15 | CUC 0.07 | CAA 0.14 | GCA 0.28 |
| AGC 0.20 | | CUU 0.04 | CCU 0.08 | CUU 0.04 | | GCG 0.26 |
| UCG 0.04 | | UUG 0.03 | CCC 0.00 | UUG 0.03 | | GCC 0.10 |
| AGU 0.03 | | UUA 0.02 | | UUA 0.02 | | |
| UCA 0.02 | | CUA 0.00 | | CUA 0.00 | | |

127-133

| Leu | Val | Met | Gly | Glu | Gly | Thr |
|---|---|---|---|---|---|---|
| CUG 0.83 | GUU 0.51 | AUG 1.00 | GGU 0.59 | GAA 0.78 | GGU 0.59 | ACC 0.55 |
| CUC 0.07 | GUA 0.26 | | GGC 0.38 | GAG 0.22 | GGC 0.38 | ACU 0.35 |
| CUU 0.04 | GUG 0.16 | | GGG 0.02 | | GGG 0.02 | ACG 0.07 |
| UUG 0.03 | GUC 0.07 | | GGA 0.00 | | GGA 0.00 | ACA 0.04 |
| UUA 0.02 | | | | | | |
| CUA 0.00 | | | | | | |

134-140

| Cys | Glu | Lys | Arg | Arg | Asp | Ala |
|---|---|---|---|---|---|---|
| UGC 0.51 | GAA 0.78 | AAA 0.74 | CGU 0.74 | CGU 0.74 | GAC 0.67 | GCU 0.35 |
| UGU 0.49 | GAG 0.22 | AAG 0.26 | CGC 0.25 | CGC 0.25 | GAU 0.33 | GCA 0.28 |
| | | | CGA 0.01 | CGA 0.01 | | GCG 0.26 |
| | | | AGG 0.00 | AGG 0.00 | | GCC 0.10 |
| | | | AGA 0.00 | AGA 0.00 | | |
| | | | CGG 0.00 | CGG 0.00 | | |
| 218 | 316 | 272 | 128 | 135 | 137 | 121 |

141-147

| Glu | Tyr | Gly | Ala | Ser | Pro | Glu |
|---|---|---|---|---|---|---|
| GAA 0.78 | UAC 0.75 | GGU 0.59 | GCU 0.35 | UCC 0.37 | CCC 0.77 | GAA 0.78 |
| GAG 0.22 | UAU 0.25 | GGC 0.38 | GCA 0.28 | UCU 0.34 | CCA 0.15 | GAG 0.22 |
| | | GGG 0.02 | GCG 0.26 | AGC 0.20 | CCU 0.08 | |
| | | GGA 0.00 | GCC 0.10 | UCG 0.04 | CCC 0.00 | |
| | | | | AGU 0.03 | | |
| | | | | UCA 0.02 | | |

148-154

| Gln | Val | Ala | Asp | Asn | Gly | Asp |
|---|---|---|---|---|---|---|
| CAG 0.86 | GUU 0.51 | GCU 0.35 | GAC 0.67 | AAC 0.94 | GGU 0.59 | GAC 0.67 |
| CAA 0.14 | GUA 0.26 | GCA 0.28 | GAU 0.33 | AAU 0.06 | GGC 0.38 | GAU 0.33 |
| | GUG 0.16 | GCG 0.26 | | | GGG 0.02 | |
| | GUC 0.07 | GCC 0.10 | | | GGA 0.00 | |

155-161

| Asp | His | Ser | Glu | Gly | Gly | Leu |
|---|---|---|---|---|---|---|
| GAC 0.67 | CAC 0.83 | UCC 0.37 | GAA 0.78 | GGU 0.59 | GGU 0.59 | CUG 0.83 |
| GAU 0.33 | CAU 0.17 | UCU 0.34 | GAG 0.22 | GGC 0.38 | GGC 0.38 | CUC 0.07 |
| | | AGC 0.20 | | GGG 0.02 | GGG 0.02 | CUU 0.04 |
| | | UCG 0.04 | | GGA 0.00 | GGA 0.00 | UUG 0.03 |
| | | AGU 0.03 | | | | UUA 0.02 |
| | | UCA 0.02 | | | | CUA 0.00 |

162-168

| Val | Glu | Asn | His | Val | Asp | Ser |
|---|---|---|---|---|---|---|
| GUU 0.51 | GAA 0.78 | AAC 0.94 | CAC 0.83 | GUU 0.51 | GAC 0.67 | UCC 0.37 |
| GUA 0.26 | GAG 0.22 | AAU 0.06 | CAU 0.17 | GUA 0.26 | GAU 0.33 | UCU 0.34 |
| GUG 0.16 | | | | GUG 0.16 | | AGC 0.20 |
| GUC 0.07 | | | | GUC 0.07 | | UCG 0.04 |
| | | | | | | AGU 0.03 |
| | | | | | | UCA 0.02 |

169-175

| Thr | Met | Asn | Met | Leu | Gly | Gly |
|---|---|---|---|---|---|---|
| ACC 0.55 | AUG 1.00 | AAC 0.94 | AUG 1.00 | CUG 0.83 | GGU 0.59 | GGU 0.59 |
| ACU 0.35 | | AAU 0.06 | | CUC 0.07 | GGC 0.38 | GGC 0.38 |
| ACG 0.07 | | | | CUU 0.04 | GGG 0.02 | GGG 0.02 |
| ACA 0.04 | | | | UUG 0.03 | GGA 0.00 | GGA 0.00 |
| | | | | UUA 0.02 | | |
| | | | | CUA 0.00 | | |

176-182

| Gly | Gly | Ser | Ala | Gly | Arg | Lys |
|---|---|---|---|---|---|---|
| GGU 0.59 | GGU 0.59 | UCC 0.37 | GCU 0.35 | GGU 0.59 | CGU 0.74 | AAA 0.74 |
| GGC 0.38 | GGC 0.38 | UCU 0.34 | GCA 0.28 | GGC 0.38 | CGC 0.25 | AAG 0.26 |
| GGG 0.02 | GGG 0.02 | AGC 0.20 | GCG 0.26 | GGG 0.02 | CGA 0.01 | |
| GGA 0.00 | GGA 0.00 | UCG 0.04 | GCC 0.10 | GGA 0.00 | AGG 0.00 | |
| | | AGU 0.03 | | | AGA 0.00 | |
| | | UCA 0.02 | | | CGG 0.00 | |

183-189

| Pro | Leu | Lys | Ser | Gly | Met | Lys |
|---|---|---|---|---|---|---|
| CCC 0.77 | CUG 0.83 | AAA 0.74 | UCC 0.37 | GCU 0.59 | AUG 1.00 | AAA 0.74 |
| CCA 0.15 | CUC 0.07 | AAG 0.26 | UCU 0.34 | GGC 0.38 | | AAG 0.26 |
| CCU 0.08 | CUU 0.04 | | AGC 0.20 | GGG 0.02 | | |
| CCC 0.00 | UUG 0.03 | | UCG 0.04 | GGA 0.00 | | |
| | UUA 0.02 | | AGU 0.03 | | | |
| | CUA 0.00 | | UCA 0.02 | | | |

190-196

| Glu | Leu | Ala | Val | Phe | Arg | Glu |
|---|---|---|---|---|---|---|
| GAA 0.78 | CUG 0.83 | GCU 0.35 | GUU 0.51 | UUC 0.76 | CGU 0.74 | GAA 0.78 |
| GAG 0.22 | CUC 0.07 | GCA 0.28 | GUA 0.26 | UUU 0.24 | CGC 0.25 | GAG 0.22 |
| | CUU 0.04 | GCG 0.26 | GUG 0.16 | | CGA 0.01 | |
| | UUG 0.03 | GCC 0.10 | GUC 0.07 | | AGG 0.00 | |
| | UUA 0.02 | | | | AGA 0.00 | |
| | CUA 0.00 | | | | CGG 0.00 | |

197-203

| Lys | Val | Thr | Glu | Gln | His | Arg |
|---|---|---|---|---|---|---|
| AAA 0.74 | GUU 0.51 | ACC 0.55 | GAA 0.78 | CAG 0.86 | CAC 0.83 | CGU 0.74 |
| AAG 0.26 | GUA 0.26 | ACU 0.35 | GAG 0.22 | CAA 0.14 | CAU 0.17 | CGC 0.25 |
| | GUG 0.16 | ACG 0.07 | | | | CGA 0.01 |
| | GUC 0.07 | ACA 0.04 | | | | AGG 0.00 |
| | | | | | | AGA 0.00 |
| | | | | | | CGG 0.00 |

204-210

| Gln | Met | Gly | Lys | Gly | Gly | Lys |
|---|---|---|---|---|---|---|
| CAG 0.86 | AUG 1.00 | GGU 0.59 | AAA 0.74 | GGU 0.59 | GGU 0.59 | AAA 0.74 |
| CAA 0.14 | | GGC 0.38 | AAG 0.26 | GGC 0.38 | GGC 0.38 | AAG 0.26 |
| | | GGG 0.02 | | GGG 0.02 | GGG 0.02 | |
| | | GGA 0.00 | | GGA 0.00 | GGA 0.00 | |

211-217

| His | His | Leu | Gly | Leu | Glu | Glu |
|---|---|---|---|---|---|---|
| CAC 0.83 | CAC 0.83 | CUG 0.83 | GGU 0.59 | CUG 0.83 | GAA 0.78 | GAA 0.78 |
| CAU 0.17 | CAU 0.17 | CUC 0.07 | GGC 0.38 | CUC 0.07 | GAG 0.22 | GAG 0.22 |
| | | CUU 0.04 | GGG 0.02 | CUU 0.04 | | |
| | | UUG 0.03 | GGA 0.00 | UUG 0.03 | | |
| | | UUA 0.02 | | UUA 0.02 | | |
| | | CUA 0.00 | | CUA 0.00 | | |

218-224

| Pro | Lys | Lys | Leu | Arg | Pro | Pro |
|---|---|---|---|---|---|---|
| CCG 0.77 | AAA 0.74 | AAA 0.74 | CUG 0.83 | CGU 0.74 | CCG 0.77 | CCG 0.77 |
| CCA 0.15 | AAG 0.26 | AAG 0.26 | CUC 0.07 | CGC 0.25 | CCA 0.15 | CCA 0.15 |
| CCU 0.08 | | | CUU 0.04 | CGA 0.01 | CCU 0.08 | CCU 0.08 |
| CCC 0.00 | | | UUG 0.03 | AGG 0.00 | CCC 0.00 | CCC 0.00 |
| | | | UUA 0.02 | AGA 0.00 | | |
| | | | CUA 0.00 | CGG 0.00 | | |

225-231

| Pro | Ala | Arg | Thr | Pro | Cys | Gln |
|---|---|---|---|---|---|---|
| CCG 0.77 | GCU 0.35 | CGU 0.74 | ACC 0.55 | CCG 0.77 | UGC 0.51 | CAG 0.86 |
| CCA 0.15 | GCA 0.28 | CGC 0.25 | ACU 0.35 | CCA 0.15 | UGU 0.49 | CAA 0.14 |
| CCU 0.08 | GCG 0.26 | CGA 0.01 | ACG 0.07 | CCU 0.08 | | |
| CCC 0.00 | GCC 0.10 | AGG 0.00 | ACA 0.04 | CCC 0.00 | | |
| | | AGA 0.00 | | | | |
| | | CGG 0.00 | | | | |

232-238

| Gln | Glu | Leu | Asp | Gln | Val | Leu |
|---|---|---|---|---|---|---|
| CAG 0.86 | GAA 0.78 | CUG 0.83 | GAC 0.67 | CAG 0.86 | GUU 0.51 | CUG 0.83 |
| CAA 0.14 | GAG 0.22 | CUC 0.07 | GAU 0.33 | CAA 0.14 | GUA 0.26 | CUC 0.07 |
| | | CUU 0.04 | | | GUG 0.16 | CUU 0.04 |
| | | UUG 0.03 | | | GUC 0.07 | UUG 0.03 |
| | | UUA 0.02 | | | | UUA 0.02 |
| | | CUA 0.00 | | | | CUA 0.00 |

239-245

| Glu | Arg | Ile | Ser | Thr | Met | Arg |
|---|---|---|---|---|---|---|
| GAA 0.78 | CGU 0.74 | AUC 0.83 | UCC 0.37 | ACC 0.55 | AUG 1.00 | CGU 0.74 |
| GAG 0.22 | CGC 0.25 | AUU 0.17 | UCU 0.34 | ACU 0.35 | | CGC 0.25 |
| | CGA 0.01 | AUA 0.00 | AGC 0.20 | ACG 0.07 | | CGA 0.01 |
| | AGG 0.00 | | UCG 0.04 | ACA 0.04 | | AGG 0.00 |
| | AGA 0.00 | | AGU 0.03 | | | AGA 0.00 |
| | CGG 0.00 | | UCA 0.02 | | | CGG 0.00 |

246-252

| Leu | Pro | Asp | Glu | Arg | Gly | Pro |
|---|---|---|---|---|---|---|
| CUG 0.83 | CCG 0.77 | GAC 0.67 | GAA 0.78 | CGU 0.74 | GGU 0.59 | CCG 0.77 |
| CUC 0.07 | CCA 0.15 | GAU 0.33 | GAG 0.22 | CGC 0.25 | GGC 0.38 | CCA 0.15 |
| CUU 0.04 | CCU 0.08 | | | CGA 0.01 | GGG 0.02 | CCU 0.08 |
| UUG 0.03 | CCC 0.00 | | | AGG 0.00 | GGA 0.00 | CCC 0.00 |
| UUA 0.02 | | | | AGA 0.00 | | |
| CUA 0.00 | | | | CGG 0.00 | | |

253-259

| Leu | Glu | His | Leu | Tyr | Ser | Leu |
|---|---|---|---|---|---|---|
| CUG 0.83 | GAA 0.78 | CAC 0.83 | CUG 0.83 | UAC 0.75 | UCC 0.37 | CUG 0.83 |
| CUC 0.07 | GAG 0.22 | CAU 0.17 | CUC 0.07 | UAU 0.25 | UCU 0.34 | CUC 0.07 |
| CUU 0.04 | | | CUU 0.04 | | AGC 0.20 | CUU 0.04 |
| UUG 0.03 | | | UUG 0.03 | | UCG 0.04 | UUG 0.03 |
| UUA 0.02 | | | UUA 0.02 | | AGU 0.03 | UUA 0.02 |
| CUA 0.00 | | | CUA 0.00 | | UCA 0.02 | CUA 0.00 |

260-266

| His | Ile | Pro | Asn | Cys | Asp | Lys |
|---|---|---|---|---|---|---|
| CAC 0.83 | AUC 0.83 | CCG 0.77 | AAC 0.94 | UGC 0.51 | GAC 0.67 | AAA 0.74 |
| CAU 0.17 | AUU 0.17 | CCA 0.15 | AAU 0.06 | UGU 0.49 | GAU 0.33 | AAG 0.26 |
| | AUA 0.00 | CCU 0.08 | | | | |
| | | CCC 0.00 | | | | |

267-273

| His | Gly | Leu | Tyr | Asn | Leu | Lys |
|---|---|---|---|---|---|---|
| CAC 0.83 | GGU 0.59 | CUG 0.83 | UAC 0.75 | AAC 0.94 | CUG 0.83 | AAA 0.74 |
| CAU 0.17 | GGC 0.38 | CUC 0.07 | UAU 0.25 | AAU 0.06 | CUC 0.07 | AAG 0.26 |
| | GGG 0.02 | CUU 0.04 | | | CUU 0.04 | |
| | GGA 0.00 | UUG 0.03 | | | UUG 0.03 | |
| | | UUA 0.02 | | | UUA 0.02 | |
| | | CUA 0.00 | | | CUA 0.00 | |

274-280

| Gln | Cys | Lys | Met | Ser | Leu | Asn |
|---|---|---|---|---|---|---|
| CAG 0.86 | UGC 0.51 | AAA 0.74 | AUG 1.00 | UCC 0.37 | CUG 0.83 | AAC 0.94 |
| CAA 0.14 | UGU 0.49 | AAG 0.26 | | UCU 0.34 | CUC 0.07 | AAU 0.06 |
| | | | | AGC 0.20 | CUU 0.04 | |
| | | | | UCG 0.04 | UUG 0.03 | |
| | | | | AGU 0.03 | UUA 0.02 | |
| | | | | UCA 0.02 | CUA 0.00 | |

281-287

| Gly | Gln | Arg | Gly | Glu | Cys | Trp |
|---|---|---|---|---|---|---|
| GGU 0.59 | CAG 0.86 | CGU 0.74 | GGU 0.59 | GAA 0.78 | UGC 0.51 | UGG 1.00 |
| GGC 0.38 | CAA 0.14 | CGC 0.25 | GGC 0.38 | GAG 0.22 | UGU 0.49 | |
| GGG 0.02 | | CGA 0.01 | GGG 0.02 | | | |
| GGA 0.00 | | AGG 0.00 | GGA 0.00 | | | |
| | | AGA 0.00 | | | | |
| | | CGG 0.00 | | | | |

288-294

| Cys | Val | Asn | Pro | Asn | Thr | Gly |
|---|---|---|---|---|---|---|
| UGC 0.51 | GUU 0.51 | AAC 0.94 | CCG 0.77 | AAC 0.94 | ACC 0.55 | GGU 0.59 |
| UGU 0.49 | GUA 0.26 | AAU 0.06 | CCA 0.15 | AAU 0.06 | ACU 0.35 | GGC 0.38 |
| | GUG 0.16 | | CCU 0.08 | | ACG 0.07 | GGG 0.02 |
| | GUC 0.07 | | CCC 0.00 | | ACA 0.04 | GGA 0.00 |

295-301

| Lys | Leu | Ile | Gln | Gly | Ala | Pro |
|---|---|---|---|---|---|---|
| AAA 0.74 | CUG 0.83 | AUC 0.83 | CAG 0.86 | GGU 0.59 | GCU 0.35 | CCG 0.77 |
| AAG 0.26 | CUC 0.07 | AUU 0.17 | CAA 0.14 | GGC 0.38 | GCA 0.28 | CCA 0.15 |
| | CUU 0.04 | AUA 0.00 | | GGG 0.02 | GCG 0.26 | CCU 0.08 |
| | UUG 0.03 | | | GGA 0.00 | GCC 0.10 | CCC 0.00 |
| | UUA 0.02 | | | | | |
| | CUA 0.00 | | | | | |

302-308

| Thr | Ile | Arg | Gly | Glu | Pro | Glu |
|---|---|---|---|---|---|---|
| ACC 0.55 | AUC 0.83 | CGU 0.74 | GGU 0.59 | GAA 0.67 | CCG 0.77 | GAA 0.78 |
| ACU 0.35 | AUU 0.17 | CGC 0.25 | GGC 0.38 | GAG 0.33 | CCA 0.15 | GAG 0.22 |
| ACG 0.07 | AUA 0.00 | CGA 0.01 | GGG 0.02 | | CCU 0.08 | |
| ACA 0.04 | | AGG 0.00 | GGA 0.00 | | CCC 0.00 | |
| | | AGA 0.00 | | | | |
| | | CGG 0.00 | | | | |

309-315

| Cys | His | Leu | Phe | Tyr | Asn | Glu |
|---|---|---|---|---|---|---|
| UGC 0.51 | CAC 0.83 | CUG 0.83 | UUC 0.76 | UAC 0.75 | AAC 0.94 | GAA 0.78 |
| UGU 0.49 | CAU 0.17 | CUC 0.07 | UUU 0.24 | UAU 0.25 | AAU 0.06 | GAG 0.22 |
| | | CUU 0.04 | | | | |
| | | UUG 0.03 | | | | |
| | | UUA 0.02 | | | | |
| | | CUA 0.00 | | | | |

316-322

| Gln | Gln | Glu | Ala | Cys | Gly | Val |
|---|---|---|---|---|---|---|
| CAG 0.86 | CAG 0.86 | GAA 0.78 | GCU 0.35 | UGC 0.51 | GGU 0.59 | GUU 0.51 |
| CAA 0.14 | CAA 0.14 | GAG 0.22 | GCA 0.28 | UGU 0.49 | GGC 0.38 | GUA 0.26 |
| | | | GCG 0.26 | | GGG 0.02 | GUG 0.16 |
| | | | GCC 0.10 | | GGA 0.00 | GUC 0.07 |

323-328

| His | Thr | Gln | Arg | Met | Gln |
|---|---|---|---|---|---|
| CAC 0.83 | ACC 0.55 | CAG 0.86 | CGU 0.74 | AUG 1.00 | CAG 0.86 |
| CAU 0.17 | ACU 0.35 | CAA 0.14 | CGC 0.25 | | CAA 0.14 |
| | ACG 0.07 | | CGA 0.01 | | |
| | ACA 0.04 | | AGG 0.00 | | |
| | | | AGA 0.00 | | |
| | | | CGG 0.00 | | |

SEQ ID NO: 6

| PQQLNP-IGFBP-2 [Homo sapiens]. | |
|---|---|
| Modified from ACCESSION NP_000588 | |
| 1 | mlprvgcpal plppppllpl lpllllllga sgggggarae vlfrcppctp |
| 51 | erlaacgppp vappaavaav aggarmpcae lvrepgcgcc svcarlegea |
| 101 | cgvytprcgq glrcyphpgs elplqalvmg egtcekrrda eygaspeqva |
| 151 | dngddhsegg lvenhvdstm nmlggggsag rkplksgmke lavfrekvte |
| 201 | qhrqmgkggk hhlgleepqg lnpppartpc qqeldqvler istmrlpder |
| 251 | gplehlyslh ipncdkhgly nlkqckmsln gqrgecwcvn pntgkliqga |
| 301 | ptirgdpech lfyneqqeac gvhtqrmq |

The figures show:

FIG. 1: The genes of senescence are evolutionary highly conserved.

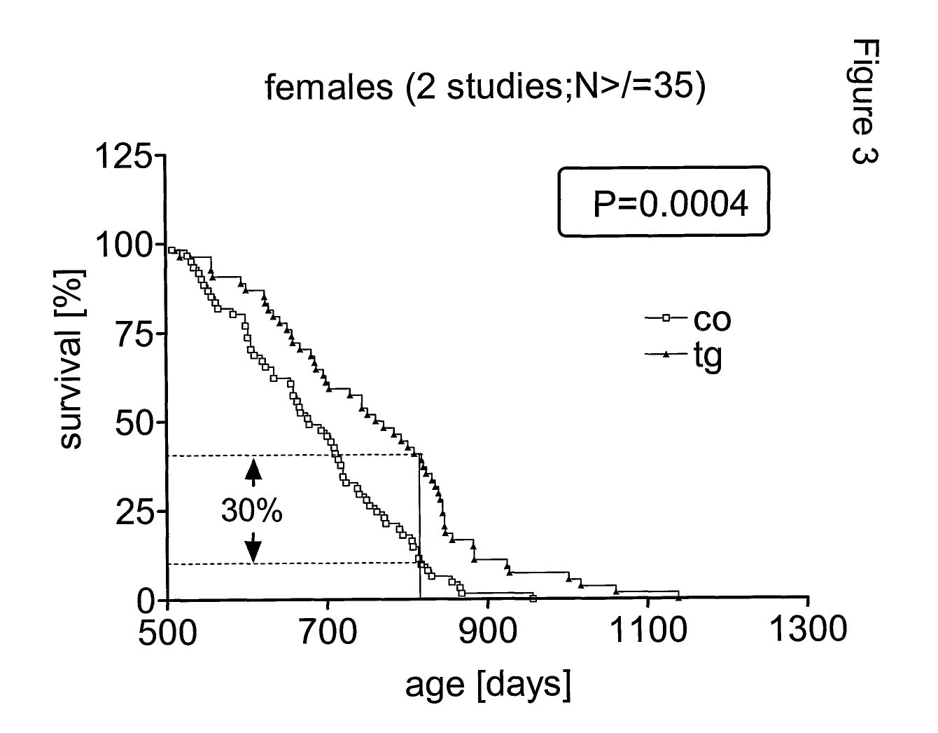

FIG. 2: Increased life expectancy in female IGFBP-2-carrying mice (closed symbols, N=20) compared to non-carrying controls (open symbols; N=22). In male mice, no effect on life expectancy has been seen up to now (C: N=5; B: N=6).

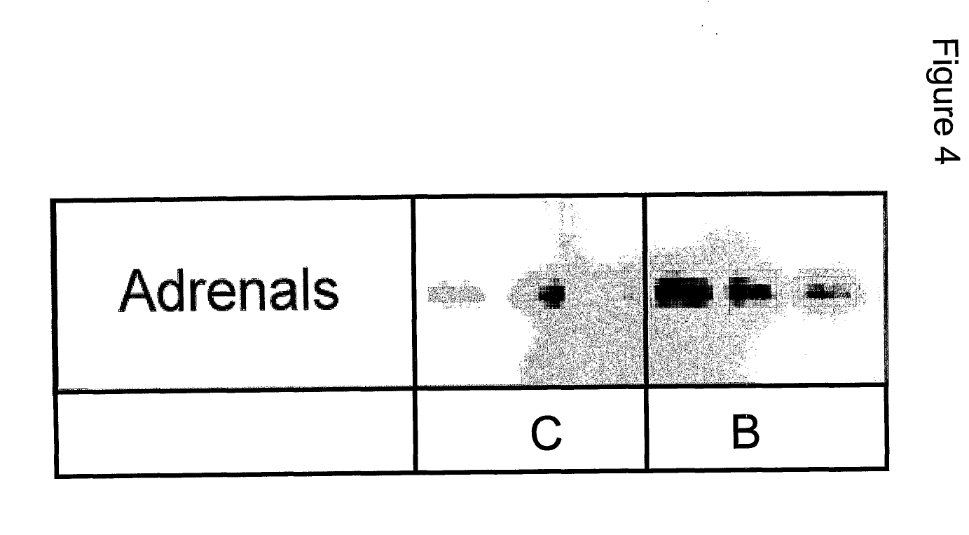

FIG. 3: Surviving curves (Kaplan-Meier Plot) of IGFBP-2 transgenic and non-transgenic mice.

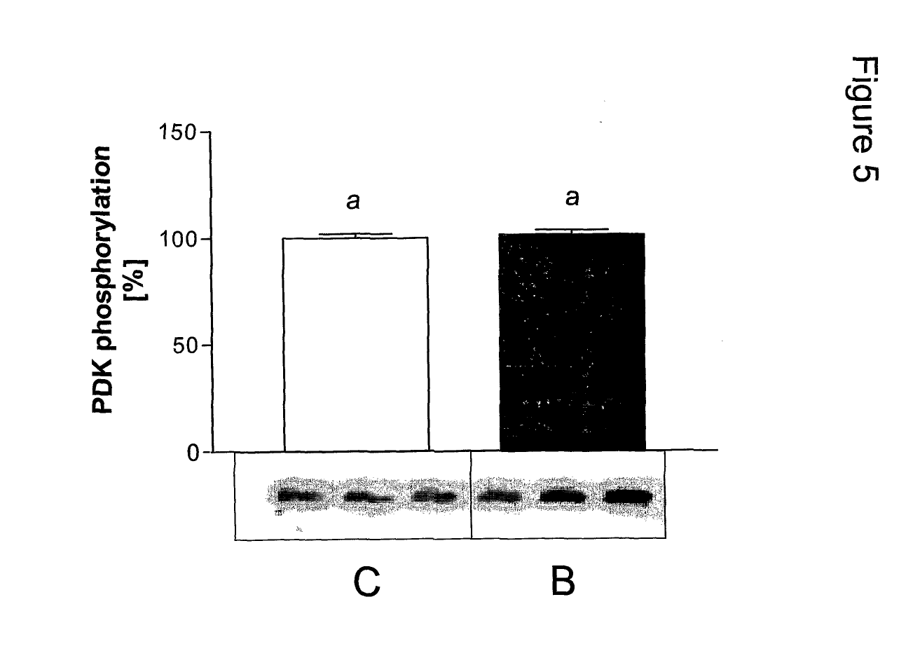

FIG. 4: Increased phosphorylation of FKHR in different tissues of IGFBP-2 transgenic mice. The results have been achieved by use of phospho-specific antibodies. The molecular weight corresponds to the theoretically predicted molecular weight.

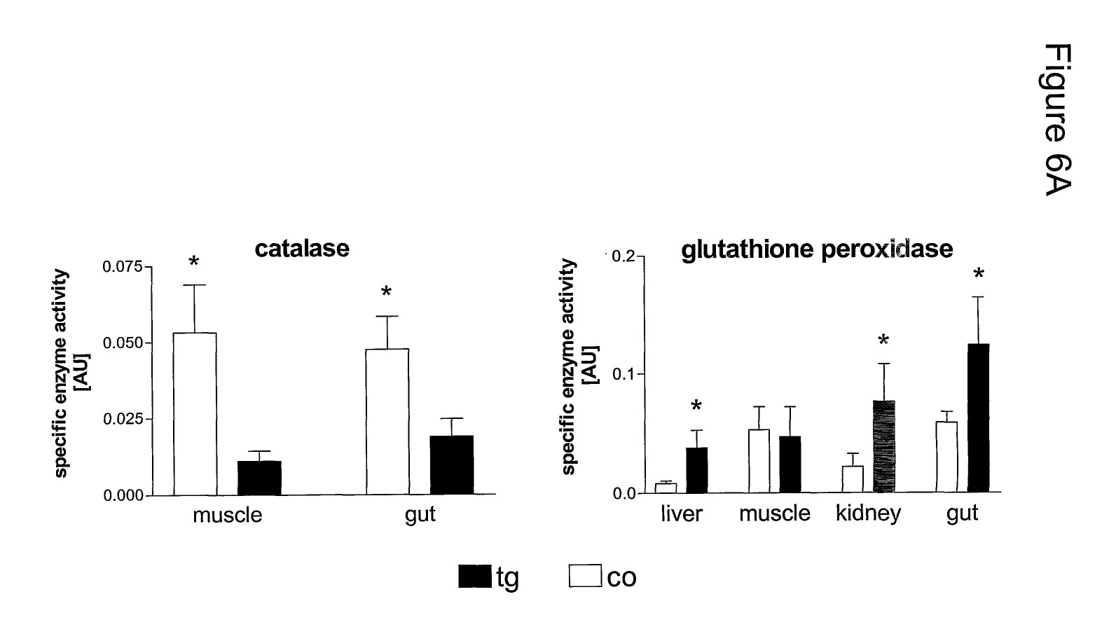

FIG. 5: The activation of PDK, an important signal molecule of the PI-3 kinase pathway is not changed in IGFBP-2 transgenic mice compared to non-transgenic brothers and sisters from the same brood.

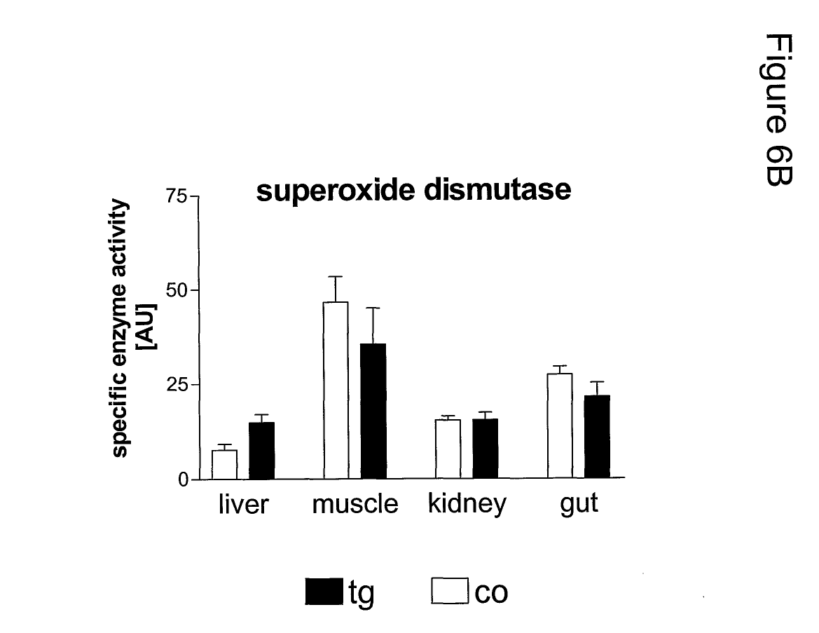

FIG. 6: Relative activities of key enzymes of the oxygen peroxide metabolism. The enzyme activities were correlated to the amount of protein and were thus referred to as specific activities.

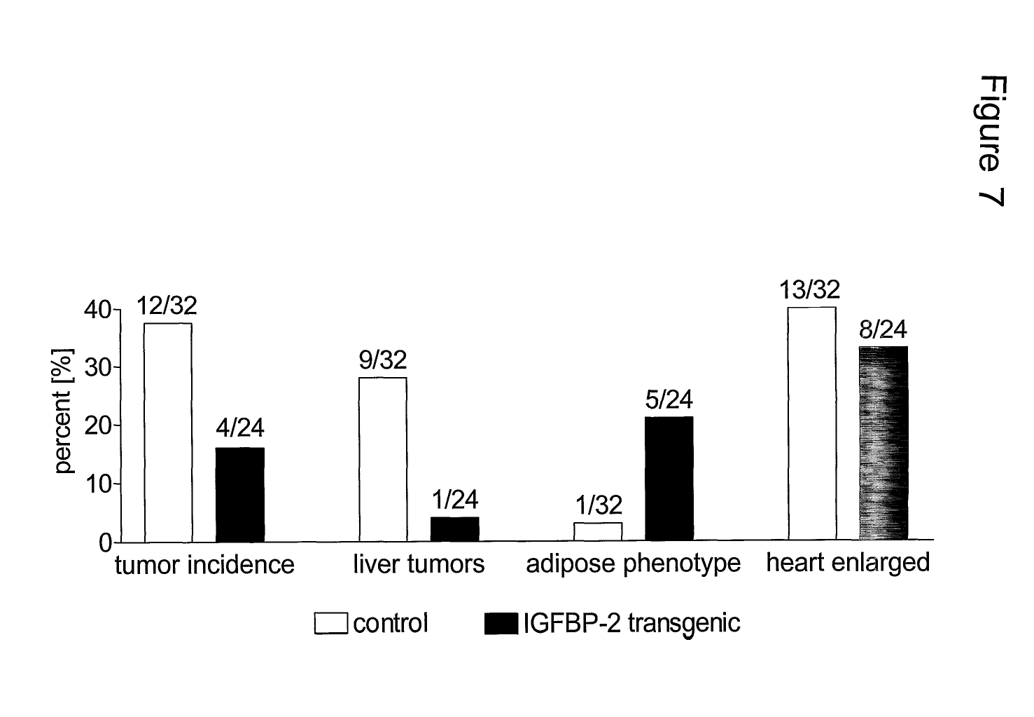

FIG. 7: Clinical examination of IGFBP-2 transgenic mice and non-transgenic controls. In IGFBP-2 transgenic mice, there is a decreased tumour incidence and indications as to an ameliorated constitution of the heart.

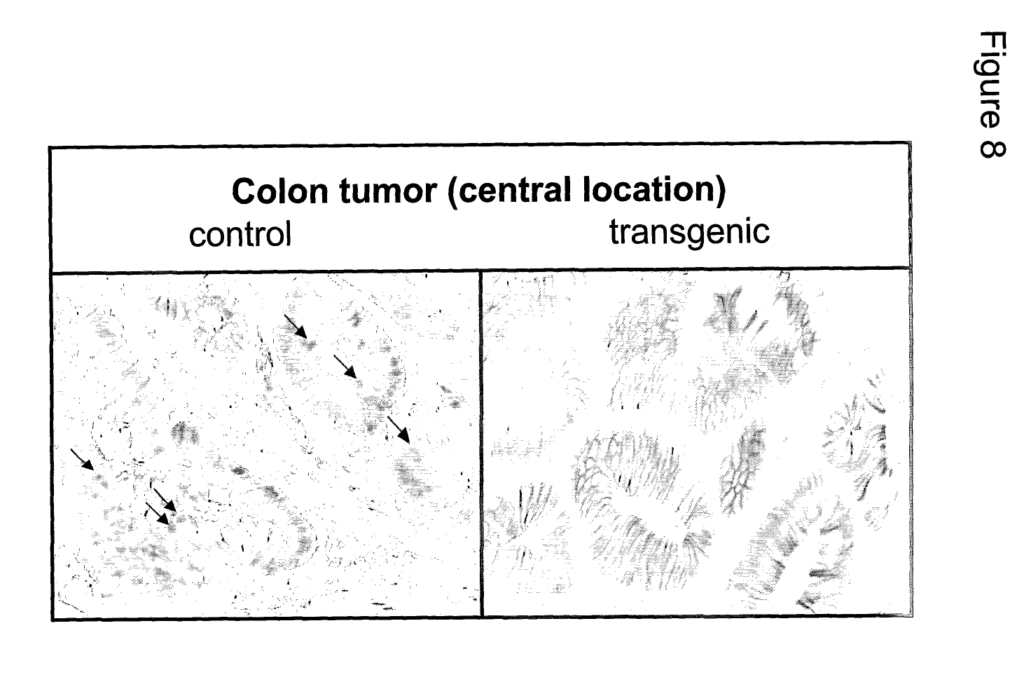

FIG. 8: IGFBP-2 overexpression in transgenic mice results a strong inhibition of nuclear β-catenin localization in colon tumours derived by chemical carcinogenesis. The arrows indicate nuclear localization of β-catenin in tumours from non transgenic mice. In IGFBP-2 transgenic mice virtually no β-catenin was present in the cell nuclei, which predominantly was found associated with the cell-membrane.

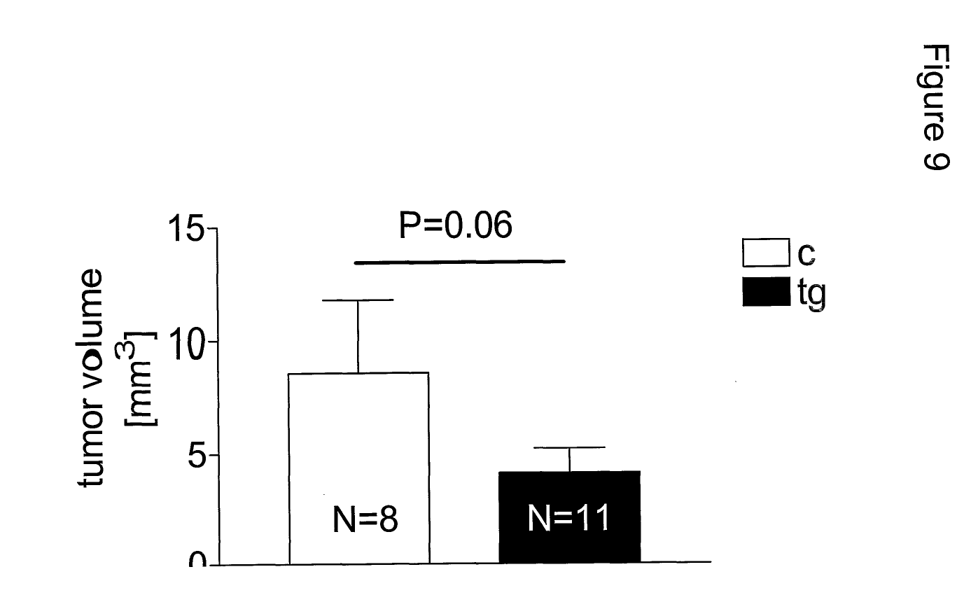

FIG. 9: IGFBP-2 overexpression in transgenic mice results strong reduction of tumour volumes after chemical carcinogenesis. This finding, as also the observation of a reduced tumour incidence in highly senescent mice, indicate tumour-protective potential of IGFBP-2 in vivo. It is worth pointing out that IGFBP-2 both normal and malignant conditions seems to have tumour protective potential.

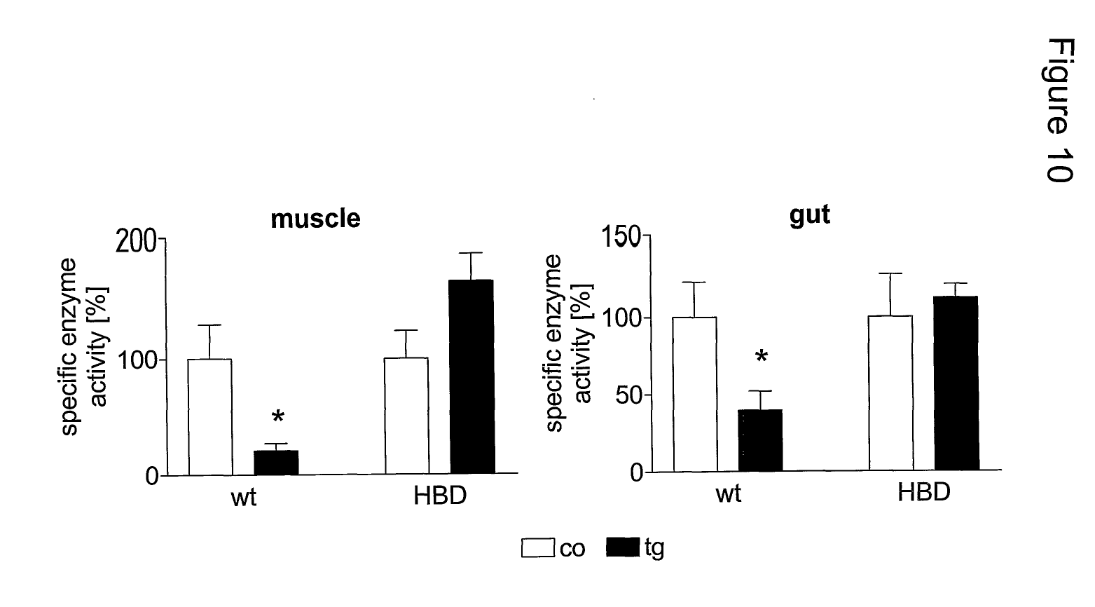

FIG. 10: In a new mouse model expressing an IGFBP-2 variant, which is characterized by reduced binding of IGFBP-2 to proteoglycans no effects on the enzyme activity of catalase can be measured. We do therefore not exclude the possibility, that IGFBP-2 exerts is effects on life span and tumour growth via proteoglycans by IGF-independent mechanisms.

The following experiments serve the illustration of the invention and are not conclusive.

### Test Animals, Material and Methods

IGFBP-2 transgenic mice are produced using a murine IGFBP-2 cDNA controlled by a CMV promoter in the C57BC/6 background; cf. Hoeflich (1999). The maximum age of IGFBP-2 transgenic mice or of non-transgenic brothers and sisters of the same brood was determined in a orientation survival study. After their natural death or after the euthanasia due to veterinary indication, the animals were dissected and were examined for pathological peculiarities.

### Western Immunoblotting

The protein fraction was isolated from different organs of adult mice and was quantified by means of bicinchonic acid/Cu—SO₄. Identical amounts of protein from the tissues were separated by SDS-PAGE, transferred to PVDF membranes and subsequently examined by Western immunoblotting as already described in detail before (Hoeflich 1998). For the analysis of activation of specific signal proteins, phospho-specific antibodies and secondary antibodies were used (Cell Signalling, New England Biolabs, Frankfurt). All dilutions and processing were according to the manufacturers instructions (Cell Signalling, New England Biolabs, Frankfurt). The detection was carried out by means of Enhanced Chemiluminescence (ECL, Amersham Biosciences, Freiburg) using the Image Station 440 CF (Kodak, Stuttgart).

### Enzyme Tests

Enzyme activity of catalase was measured in cell lysates by monitoring decomposition of 10 mM hydrogen peroxide in 60 mM sodium phosphate buffered solution (pH 7.0) at 240 nm according to the method described previously (Aebi 1984).

Glutathione peroxidase activity was measured using a commercial colorimetric assay (IBL, Hamburg, Germany). One unit is defined as the amount of enzyme that will cause oxidation of 1.0 mmol of NAPDH to NADP+ per min at 25° C.

Enzyme activity of SOD was measured according to the method of Madesh and Balasubramanian (1998). In brief, tissue lysates (10 μg protein in 20 μl of PBS) were incubated in microtiter plates with assay solution containing 15 μl pyrogallol (100 μM), 6 μl of MTT (1.25 mM) and 109 μl PBS. After 5 min at room temperature the reaction was terminated by the addition of 150 μl dimethyl sulfoxide and the absorbance was measured at 570 nm and 630 nm as the reference wavelength. A standard curve was generated using purified SOD at concentrations up to 100 ng per reaction instead of the sample.

### Immunohistochemistry

In order to analyze potential effects of IGFBP-2 on β-catenin localization in a tumour model we have used a model of chemical carcinogenesis in the colon according to standard procedures (DMH induced colon cancer; Jackson 2003). IGFBP-2 transgenic and non transgenic mice were euthanized after DMH treatment at an age of 34 weeks and tumours were isolated from the colon isolated. Tumour volumes were measured using a calliper and calculated according to a standard formula (ellipsoid model: 4/3π*r³). The tissues were fixed for 24 hours in paraformaldehyde (PFA; 4% PFA in phosphate buffered solution, pH=7.4). Sections were performed from paraffin embedded tumour material at a nomnal thickness of 4 μm using a microtome (Rotations Microtom, Leica, Modell Mulicut 2045). The sections were then used for immunohistochemical detection of α-catenin. Thus, the sections were deparaffinized, rehydrated and blocked in 5% goat serum and blocking buffer (Avidin/Biotin-Blocking Kit; Vector Laboratories). The sections were then incubated with diluted (1/10000) antibody (anti-β-catenin, BD Transduction Laboratories, Lexington, Cat No: 610153) overnight at room temperature. After three washings in Tris-buffered saline, the sections were incubated with diluted (1/200) secondary antibody (biotinylated anti-mouse IgG Vector Laboratories Burlingame BA-9200) for 1 hour at room temperature. After three washings as indicated above, β-catenin was visualized using the ABC-Kit (Vector Laboratories) according to the manufacturers instructions.

In order to characterize the mechanism of action relevant for the prolonged life span in IGFBP-2 transgenic mice and thereby to exclude an IGF-dependent mechanism of action a new mouse model (heparin-binding-defficient-IGFBP-2; HBD-IGFBP-2) expressing an IGFBP-2 variant was established. In the new mouse model IGFBP-2 was mutated in order to block interaction of IGFBP-2 with proteoglacans (PKKLRP->PNNLAP; Russo 2005). Since the effect of IGFBP-2 on life span can be measured in accordance with this invention through affecting the activity of redox-relevant enzymes the activity of catalase in HBD-IGFBP-2 transgenic mice was analyzed.

### Results and Discussion

Female IGFBP-2 transgenic animals reached a significantly higher age than their non-transgenic brothers and sisters from the same brood (FIG. 2). The absolute increase in mean life expectancy amounts to approx. 2.5 months and is approx. 10%. At a point in time where only approx. 25% of the control animals are still alive, a majority of the IGFBP-2 transgenic female mice (FIG. 3) is still alive. As is known, FKHR represents an important control element in the regulation of the senescence. According to today's knowledge, the activation can be carried out by the PI3 kinase pathway, by p66Shc or as shown recently by β-catenin. In order to include the activation by alternate signalling cascades, the phosphorylation of FKHR was examined in adrenal glands. Substantial increases of FKHR phosphorylation in adrenal glands were detected; see FIG. 4. In order to reconstruct the activation pathway, further components of the PI-3 kinase signal pathway were analysed (FIG. 5). Upon examination of the MAPK and PI-3 kinase pathways, no changes in the protein phosphorylation could be detected. Hereby, an activation of the PI3 kinase pathway under increased serum and tissue levels of IGFBP-2 could not be detected in the transgenic animal model.

FKHR exerts a part of its effects on senescence via the control of redox-relevant enzymes. In particular the catalase activity is directly controlled by FKHR. Thus, the enzymatic activities of catalase, glutathione peroxidase and superoxide dismutase were also examined in different tissues.

Significant differences in the activities of the catalase and of glutathione peroxidase, but not of superoxide dismutase could be determined (FIG. 6). Without being linked to this theory, these changes can well be causally linked to the changed FKHR phosphorylation. In particular the significant change of the enzymatic activity of the catalase point to an effect of FKHR.

Upon dissection of the animals, differences between the groups could be seen with respect to the decrease of tumour incidence in the IGFBP-2 transgenic animals (31% in C; 17% in B; FIG. 7). In six of a total of 19 non-transgenic mice, tumours were found (once, each, in the pancreas, in the skin and in the ovaries, four times in the liver). However, in 4 of 24 transgenic mice tumours occurred in brain, intestine, larynx, liver, muscle and connective tissue (once, each). The tumour incidence in the liver and the ovaries is, thus, significantly reduced. IGFBP-2 can, depending on the organ or the condition of the cell, impede or further the rate of cell division. Thus, a conditional effect could be shown in vitro, wherein IGFBP-2 can impede cell division in non-malignant cells, whereas in malignant cells the cell division can be stimulated (Hoeflich 2000; Moore 2003; Hoeflich 1998). In a great number of malignant diseases in humans, the serum level of IGFBP-2 is increased and also correlates with the corresponding specific tumour markers (Hoeflich 2001). Nevertheless, the IGFBP-2 concentration in blood is not indicative of an increased tumour risk and has no prognostic value (Lukanova 2004). On the contrary, with respect to ovarian cancer, even a protective role of IGFBP-2 was discussed (Lukanova 2003). However, a protective effect of IGFBP-2 for malignant diseases of the liver has until now not yet been speculated on.

Part of the IGFBP-2 transgenic animals moreover showed a clear accumulation of body fat ( 5/24) and organ increases of the spleen ( 2/24). With increasing age, the adipose phenotype of the IGFBP-2 transgenic mice was substituted by a cachectic phenotype. An effect from IGFBP-2 on energy balance can further be assumed by our results in HBD-IGFBP-2 transgenic mice (see below). A blocking of the GH/IGF-I signal pathway can increase life expectancy (Brown-Borg et al).

Data provided herein document that nuclear localization of β-catenin in colon tumours is potently blocked by IGFBP-2 (FIG. 8). In tumours from IGFBP-2 transgenic mice β-catenin is present in a membrane bound form, whereas nuclear β-catenin can not be detected. By contrast in controls nuclear β-catenin is present (arrows in FIG. 8) in addition to membrane bound β-catenin. Accordingly, IGFBP-2 has a strong impact on the expression of Wnt-signalling dependent target genes. IGFBP-2 might thus represent a novel control factor of the Wnt-signalling pathway. The effect of IGFBP-2 on the expression and activity of redox-relevant genes might be exerted via interaction with the Wnt-signalling pathway or particularly via interaction with β-catenin. As a clear protective effect for tumour growth, reduced tumour volumes in IGFBP-2 transgenic mice (FIG. 9) were detected. Therefore, IGFBP-2 is both tumour protective through both IGF-dependent and IGF-independent mechanisms. IGFBP-2 represents a multifunctional protective protein which blocks tumour growth and ageing at multiple positions.

The here present results also suggest a novel mechanism for IGFBP-2: it cannot be excluded the possibility that the effect of IGFBP-2 on the activity and expression of redox-relevant enzymes is dependent on cell surface interaction of IGFBP-2 (e.g. through interaction of IGFBP-2 with heparin or proteoglycans). When interaction of IGFBP-2 is blocked with the cell surface or with specific components from the extracellular matrix, a different phenotype for the activity of catalase is observed (FIG. 10). While wildtype IGFBP-2 in transgenic mice has a strong effect e.g. for the activity of catalase, HBD-IGFBP-2 does not effect enzyme activity of catalase in muscle or in the gastro-intestinal tract. Thus, the effect on the activity of redox-relevant enzymes seems to depend on interactions between IGFBP-2 and cell surface proteins. Hoeflich, 2001 showed that the effects of IGFBP-2 on carcass weight are IGF-dependent. These negative growth effects were stronger in HBD-mice if compared to wildtype-IGFBP-2 transgenic mice. Contrasting the effects on body or organ growth, no effects on the activity of catalase were found in HBD-IGFBP-2 transgenic mice. The effects on catalase activity and potentially on life-expectancy (which dependent on cell surface interaction) are independent from the IGFs as documented herein.

Due to the data presented herein, it can be speculated, without being bound to the theory, that IGFBP-2 regulates FKHR, redox-relevant enzymes, life expectancy and energy balance by IGF-independent mechanisms.

Until now, the use of IGFBP-2 could not be seriously considered because the malignant potential of IGFBP-2 raised doubts about the benefit for the general condition. Instead, our data demonstrate that surprisingly the opposite is true: under normal (in highly senescent mice) and under malignant conditions (during chemical carcinogenesis) IGFBP-2 turns out to be protective for tumour growth or tumour incidence. The malignant potential of IGFBP-2 in advanced cancer can, however, be decreased or eliminated in a controlled manner. As it could be shown that the interaction of IGFBP-2 with integrins can modulate the intracellular signalling (Schueft 2004; Pereira 2004), or can even have a malignant potential, in particular these possibilities of interaction of IGFBP-2 with compounds e.g. present in the plasma membrane might be manipulated. Moreover, interaction of IGFBP-2 with integral plasma membrane proteins are known (Russo 2005), which can be affected via a PKKLRP sequence. Those possibilities of interaction and further downstream pathways, too, can be manipulated e.g. by targeted mutation without affecting the protective properties and these variants or derivatives from IGFBP-2 can, thus, be used in the uses and methods presented herein.

In any case (wild-type IGFBP-2, IGFBP-2 variants, IGFBP-2 fragments or IGFBP-2 derivatives), it is particularly attractive to apply IGFBP-2 externally to e.g. fight the aging of the skin. The skin is, due to its easy accessibility, very suitable for a treatment with IGFBP-2 or IGFBP-2 variants. Indications for IGFBP-2-containing dermatologic creams would, inter alia, be the prevention of signs of senescence, psoriasis, wound healing or inflammatory skin diseases. For the therapy to be successful, IGFBP-2 might possibly not have to reach the inside of the cell but it could be decisive for the effect that IGFBP-2 is present in soluble form at the cell surface. Apart from the external use, an internal (systemic) use is also indicated due to the data presented herein. For example in order to prevent liver tumours, diseases of inner organs, e.g. of the heart or the kidney. A positive effect on the immune cells and bones can also be stated. It is of particular importance to use IGFBP-2 in order to maintain neuronal and cognitive functions. IGFBP-2 has also a positive effect on energy balance or food intake, also with respect to a mechanism for increased life expectancy as documented in this invention.

### FURTHER REFERENCES

Aebi (1984) Methods Enzymol 105, 121-126
Alexander (2000) Nat Genet. 25, 329-332
Ali (2001) Chem. Rev. 101, 2527-2540
Barbieri (2003) Am. J. Physiol. Endocrinol. Metab. 285, E1064-E1071
Besnard (2001) Biochim. Biophys. Acta 1538, 47-58
Brewer (1988), Biochem. Biophys. Res. Commun. 152, 1289-1297
Brown-Borg (2004), Ann. N.Y. Acad. Sci. 1019, 317-320
Capurro (2005) Cancer Res. 65, 6245-6254
De Meyts (2002) Nat. Rev. 23, 824-854
Dorn (2005) J. Clin. Invest. 115, 527-537
Essers (2005) Science 308, 1181-1184
Firth (2002) Endocr. Rev. 23, 824-854
Fowlkes (1997) Endocrinology 138, 2280-2285
Ghosh (2003) Nat. Rev. Mol. Cell. Biol. 4, 202-212
Hamet (2003) Metabolism 52, 5-9
Han (2005) Development 132, 667-679
Hansen (2005) PloS. Genet. 1, e17
Hekimi (1998) Trends. Genet. 14, 14-20
Hoeflich (2000) Cancer Res. 60, 834-838
Hoeflich (2003) Horm. Metab. Res. 35, 816-821
Hoeflich (2001) Cancer Res. 61, 8601-8610
Hoeflich (1998) Growth Horm. IGF. Res. 8, 113-123
Hoeflich (1999) Endocrinology 140, 5488-5496
Hoeflich (1998) FEBS Lett. 434, 329-334
Holzenberger (2003) Nature 421, 182-187
Houthoofd (2003) Exp. Gerontol 38, 947-954
Irving (1995) Exp. Cell Res. 217, 419-427
Irwin (1998) Growth Horm. IGF. Res. 8, 21-31
Jackson (2003) Carcinogenesis 24, 527-533
Lakowski (1998) Proc. Natl. Acad. Sci. USA 95,13091-13096
LeRoith (1996) Baillieres Clin. Endocrinol. Metab. 10, 49-73
Logan (2004) Annu. Rev. Cell Dev. Biol. 20, 781-810
Lukanova (2003) Cancer Causes Control 14, 285-292
Lukanova (2004) Int. J. Cancer 108, 262-268
Mark (2005) Biochemistry 44, 3644-3652
Madesh (1998) Indian J. Biochem. Biophys. 35, 184-188
Moore (2003) Int. J. Cancer 105, 14-19
Napoli (2003) Proc. Natl. Acad. Sci. U.S.A. 100, 2112-2116
Novak (1998) Proc. Natl. Acad. Sci. USA 95, 4374-4379
Parker (1998) Mol. Biol. Cell 9, 2383-2392
Pereira (2004) Cancer Res. 64, 977-984
Purdom (2003) Biogerontology 4, 181-191
Reya (2005) Nature 434, 843-850
Russo (1997) Endocrinology 138, 4858-4867
Russo (2005) Endocrinology epub
Schambony (2004) Differentiation 72, 307-318
Schuett (2004) J. Mol. Endocrinol. 32, 859-868
Song (2003) Proc. Natl. Acad. Sci. U.S.A. 100, 13970-13975
Song (2005) J. Biol. Chem. 280, 2116-2125
van den Beld (2003) Eur. J. Endocrinol. 148 627-634
Wang (2003) Cancer Res. 63, 4315-4321
Weindruch (1986) J. Nutr. 116, 641-654

1. A method of regulation of senescence processes in cells, tissues and/or organs for the maintenance of tissue and/or organ functions and/or for the treatment or alleviation of senescence symptoms or early senescence, comprising administering to a subject in need thereof a pharmaceutical composition comprising an IGFBP-2 (insulin-like growth factor binding protein 2) molecule,
wherein the IGFBP-2 molecule is selected from the group of
an IGFBP-2 polypeptide or a functional fragment thereof and
a nucleic acid encoding an IGFBP-2 polypeptide or a functional fragment or derivative thereof.

2. The method according to claim 1, wherein the IGFBP-2 polypeptide is selected from the group consisting of
(a) a polypeptide comprising an amino acid sequence as shown in SEQ ID NO:2, 4 or 6;
(b) a polypeptide which is encoded by a nucleic acid as shown in SEQ ID NO:1, 3 or 5; and
(c) a polypeptide fragment or polypeptide derivative of the polypeptides (a) or (b), wherein in this polypeptide derivative one or more amino acid residue(s) is/are conservatively exchanged and wherein this fragment or derivative can effect the same regulation of senescence processes in cells, tissues and/or organs as the polypeptide (a) or (b) and/or wherein this fragment or derivative leads to the same maintenance of tissue and/or organ functions as the polypeptide (a) or (b).

3. The method according to claim 1, wherein the nucleic acid encoding an IGFBP-2 polypeptide or a functional fragment or derivative thereof is selected from the group consisting of:
(a) a nucleic acid encoding a polypeptide with the amino acid sequence as shown in SEQ ID NO:2, 4 or 6;
(b) a nucleic acid with the coding sequence as shown in SEQ ID NO:1, 3 or 5;
(c) a nucleic acid encoding a fragment or derivative of a polypeptide which is encoded by nucleic acid according to (a) or (b), wherein in the derivative one or more amino acid residue(s) is/are conservatively exchanged and wherein the fragment or derivative can effect the same regulation of senescence processes in cells, tissues and/or organs as the polypeptide encoded by (a) or (b) and/or wherein the fragment or derivative leads to the same maintenance of tissue and/or organ functions as the polypeptide encoded by (a) or (b);
(d) a nucleic acid which is at least 80% identical to a polynucleotide as defined in (a) to (c) and which encodes a polypeptide or fragment or derivative thereof which can effect the same regulation of senescence processes in cells, tissues and/or organs as an IGFBP-2 polypeptide and/or which leads to the same maintenance of tissue and/or organ functions; and
(e) a nucleic acid whose complementary strand hybridises to a nucleic acid as defined under (a) to (d) under stringent conditions and encodes a polypeptide or a fragment or derivative thereof which can effect the same regulation of senescence processes in cells, organs and/or tissues as an IGFBP-2 polypeptide and/or which leads to the same maintenance of organ and/or tissue functions.

4. The method of claim 1, wherein said pharmaceutical composition comprises a vector containing a polypeptide or a nucleic acid as defined in claim 3.

5. The method of claim 1, wherein said pharmaceutical composition comprises a host cell which has been genetically modified with a polynucleotide or a nucleic acid as defined in claim 3 or which contains a vector as defined in claim 4.

6. The method according to claim 1, wherein the regulation of the senescence processes in cells, organs and/or tissues comprises the slowing down of an senescence process in the cells, organs and/or tissues.

7. The method according to claim 1, wherein the cells, tissues and/or organs are derived from the following organs or are the following organs: liver, heart, kidney, lung, brain, peripheral nervous systems (peripheral nervous cells), eyes, ears, stomach, intestine, connective and supporting tissue, bones and skin.

8. The method according to claim 7, wherein the cells are skin cells and the organ is the skin.

9. The method according to claim 7, wherein the cells are heart cells and the organ is the heart.

10. The method according to claim 1, wherein the cells are kidney cells and the organ is the kidney.

11. The method according to claim 1, wherein the cells are nervous cells and the organ is the brain or the bone marrow.

12. The method according to claim 1, wherein the regulation of senescence processes in cells, organs and/or tissues leads to a higher resistance against oxidative stress.

13. The method according to claim 1, wherein the maintenance of organ and/or tissue functions is the maintenance of the heart function, the function of the intestinal tract, the kidney function, the lung function, the bone function, the skin function, the function of the reproductive tract or the function of the central and/or peripheral nervous system.

14. The method according to claim 1, wherein the maintenance of the organ and/or tissue function in the heart is used for the treatment, prevention and/or therapy of a heart disease.

15. The method according to claim 14, wherein the heart disease is a heart insufficiency or a heart attack.

16. The method of claim 1, wherein said maintenance of organ and/or tissue function is the prevention, the amelioration and/or the treatment of a proliferative disorder and/or a cancerous disease.

17. The method of claim 16, wherein said prevention, amelioration and/or treatment of a proliferative disorder is the prevention, amelioration and/or treatment of cancer.

18. The method of claim 17 wherein said prevention, amelioration and/or treatment of a proliferative disorder comprises the prevention, amelioration and/or treatment of lung cancer, cancer of the reproductive tract, prostate cancer, ovarian cancer, bone cancer, kidney cancer, cancer of the intestinal tract, stomach cancer or cancer of the supporting or connective tissue.

19. The method of claim 18, wherein said cancer of the intestinal tract is colon cancer.

20. The method of claim 1, wherein said maintenance of organ and/or tissue function is the maintenance and/or restoration of body mass and/or body fat.

21. The method of claim 20, wherein said maintenance and/or restoration of body mass and/or body fat is the maintenance and/or restoration in cachexic patients and/or patients suffering from cachexia.

22. The method of claim 21, whereby said patients suffering from cachexia are cancer patients, AIDS patients, patients suffering from a metabolic disease or patients suffering from an eating disorder, from infectious diseases, from psychological disorders, or from intoxications.

23. The method according to claim 1, wherein the treatment or alleviation of senescence symptoms or early senescence comprises the treatment of skin diseases and skin senescence, the treatment of a kidney disease, the treatment of a heart disease, the treatment of a disease of the central and/or peripheral nervous system and/or the treatment of a bone disease.

24. The method according to claim 23, wherein the kidney disease is a kidney insufficiency and the heart disease is a heart insufficiency.

25. The method according to claim 24, wherein the disease of the central and/or peripheral nervous system is Alzheimer's disease, a Parkinson's disease, a dementia, an AIDS dementia, a motor neuron disease, an amyotrophic lateral sclerosis or a neurofibromatosis (Recklinghausen's disease).

26. A method of treatment
(a) for the treatment and/or alleviation of senescence symptoms;
(b) for the treatment of early senescence of cells, tissues and/or organs and/or organisms and/or
(c) for the maintenance of tissue and/or organ functions,
wherein the method of treatment comprises the administration of a therapeutic amount of an IGFBP-2 molecule as defined in claim 1, of a vector as defined in claim 4, or a host cell as defined in claim 5 to a patient to be treated.

27. The method of treatment according to claim 26, wherein the patient is a mammal.

28. The method of treatment according to claim 27, wherein the mammal is human.

28. A method of treatment
(a) for the treatment and/or alleviation of senescence symptoms;
(b) for the treatment of early senescence of cells, tissues and/or organs and/or organisms and/or
(c) for the maintenance of tissue and/or organ functions,
comprising administering a therapeutic amount of an IGFBP-2 molecule as defined in claim 2, of a vector as defined in claim 4, or a host cell as defined in claim 5 to a patient to be treated.

30. Methods of treatment
(a) for the treatment and/or alleviation of senescence symptoms;
(b) for the treatment of early senescence of cells, tissues and/or organs and/or organisms and/or
(c) for the maintenance of tissue and/or organ functions,
comprising administering a therapeutic amount of an IGFBP-2 molecule as defined in claim 3, of a vector as defined in claim 4, or a host cell as defined in claim 5 to a patient to be treated.

31. The method according to claim 2, wherein the regulation of the senescence processes in cells, organs and/or tissues comprises the slowing down of a senescence process in the cells, organs and/or tissues.

32. The method according to claim 3, wherein the regulation of the senescence processes in cells, organs and/or tissues comprises the slowing down of a senescence process in the cells, organs and/or tissues.

33. The method according to claim 2, wherein the cells, tissues and/or organs are derived from the following organs or are the following organs: liver, heart, kidney, lung, brain, peripheral nervous systems (peripheral nervous cells), eyes, ears, stomach, intestine, connective and supporting tissue, bones and skin.

34. The method according to claim 3, wherein the cells, tissues and/or organs are derived from the following organs or are the following organs: liver, heart, kidney, lung, brain, peripheral nervous systems (peripheral nervous cells), eyes, ears, stomach, intestine, connective and supporting tissue, bones and skin.

35. The method according to claim 2, wherein the maintenance of organ and/or tissue functions is the maintenance of the heart function, the function of the intestinal tract, the kidney function, the lung function, the bone function, the skin function, the function of the reproductive tract or the function of the central and/or peripheral nervous system.

36. The method according to claim 3, wherein the maintenance of organ and/or tissue functions is the maintenance of the heart function, the function of the intestinal tract, the kidney function, the lung function, the bone function, the skin function, the function of the reproductive tract or the function of the central and/or peripheral nervous system.

37. The method according to claim 2, wherein the treatment or alleviation of senescence symptoms or early senescence comprises the treatment of skin diseases and skin senescence, the treatment of a kidney disease, the treatment of a heart disease, the treatment of a disease of the central and/or peripheral nervous system and/or the treatment of a bone disease.

38. The method according to claim 3, wherein the treatment or alleviation of senescence symptoms or early senescence comprises the treatment of skin diseases and skin senescence, the treatment of a kidney disease, the treatment of a heart disease, the treatment of a disease of the central and/or peripheral nervous system and/or the treatment of a bone disease.


SEQUENCE LISTING (6 sequences)

SEQ ID NO: 1 - DNA, 1433 bp, Homo sapiens
attcggggcgagggaggaggaagaagcggaggaggcggctcccgctcgcagggccgtgcacctgcccgcccgcccgctcgctcgctcgcccgccgcgccgcgctgccgaccgccagcatgctgccgagagtgggctgccccgcgctgccgctgccgccgccgccgctgctgccgctgctgccgctgctgctgctgctactgggcgcgagtggcggcggcggcggggcgcgcgcggaggtgctgttccgctgcccgccctgcacacccgagcgcctggccgcctgcgggcccccgccggttgcgccgcccgccgcggtggccgcagtggccggaggcgcccgcatgccatgcgcggagctcgtccgggagccgggctgcggctgctgctcggtgtgcgcccggctggagggcgaggcgtgcggcgtctacaccccgcgctgcggccaggggctgcgctgctatccccacccgggctccgagctgcccctgcaggcgctggtcatgggcgagggcacttgtgagaagcgccgggacgccgagtatggcgccagcccggagcaggttgcagacaatggcgatgaccactcagaaggaggcctggtggagaaccacgtggacagcaccatgaacatgttgggcgggggaggcagtgctggccggaagcccctcaagtcgggtatgaaggagctggccgtgttccgggagaaggtcactgagcagcaccggcagatgggcaagggtggcaagcatcaccttggcctggaggagcccaagaagctgcgaccaccccctgccaggactccctgccaacaggaactggaccaggtcctggagcggatctccaccatgcgccttccggatgagcggggccctctggagcacctctactccctgcacatccccaactgtgacaagcatggcctgtacaacctcaaacagtgcaagatgtctctgaacgggcagcgtggggagtgctggtgtgtgaaccccaacaccgggaagctgatccagggagcccccaccatccggggggaccccgagtgtcatctcttctacaatgagcagcaggaggcttgcggggtgcacacccagcggatgcagtagaccgcagccagccggtgcctggcgcccctgccccccgcccctctccaaacaccggcagaaaacggagagtgcttgggtggtgggtgctggaggattttccagttctgacacacgtatttatatttggaaagagaccagcaccgagctcggcacctccccggcctctctcttcccagctgcagatgccacacctgctccttcttgctttccccgggggaggaagggggttgtggtcggggagctggggtacaggtttggggagggggaagagaaatttttatttttgaacccctgtgtcccttttgcataagattaaaggaaggaaaagt

SEQ ID NO: 2 - protein, 328 aa, Homo sapiens
MLPRVGCPALPLPPPPLLPLLPLLLLLLGASGGGGGARAEVLFRCPPCTPERLAACGPPPVAPPAAVAAVAGGARMPCAELVREPGCGCCSVCARLEGEACGVYTPRCGQGLRCYPHPGSELPLQALVMGEGTCEKRRDAEYGASPEQVADNGDDHSEGGLVENHVDSTMNMLGGGGSAGRKPLKSGMKELAVFREKVTEQHRQMGKGGKHHLGLEEPKKLRPPPARTPCQQELDQVLERISTMRLPDERGPLEHLYSLHIPNCDKHGLYNLKQCKMSLNGQRGECWCVNPNTGKLIQGAPTIRGDPECHLFYNEQQEACGVHTQRMQ

SEQ ID NO: 3 - DNA, 1433 bp, Homo Sapiens
attcggggcgagggaggaggaagaagcggaggaggcggctcccgctcgcagggccgtgcacctgcccgcccgcccgctcgctcgctcgcccgccgcgccgcgctgccgaccgccagcatgctgccgagagtgggctgccccgcgctgccgctgccgccgccgccgctgctgccgctgctgccgctgctgctgctgctactgggcgcgagtggcggcggcggcggggcgcgcgcggaggtgctgttccgctgcccgccctgcacacccgagcgcctggccgcctgcgggcccccgccggttgcgccgcccgccgcggtggccgcagtggccggaggcgcccgcatgccatgcgcggagctcgtccgggagccgggctgcggctgctgctcggtgtgcgcccggctggagggcgaggcgtgcggcgtctacaccccgcgctgcggccaggggctgcgctgctatccccacccgggctccgagctgcccctgcaggcgctggtcatgggcgagggcacttgtgagaagcgccgggacgccgagtatggcgccagcccggagcaggttgcagacaatggcgatgaccactcagaaggaggcctggtggagaaccacgtggacagcaccatgaacatgttgggcgggggaggcagtgctggccggaagcccctcaagtcgggtatgaaggagctggccgtgttccgggagaaggtcactgagcagcaccggcagatgggcaagggtggcaagcatcaccttggcctggaggagcccaagaagctgcgaccaccccctgccaggactccctgccaacaggaactggaccaggtcctggagcggatctccaccatgcgccttccggatgagcggggccctctggagcacctctactccctgcacatccccaactgtgacaagcatggcctgtacaacctcaaacagtgcaagatgtctctgaacgggcagcgtggggagtgctggtgtgtgaaccccaacaccgggaagctgatccagggagcccccaccatccggggggaacccgagtgtcatctcttctacaatgagcagcaggaggcttgcggggtgcacacccagcggatgcagtagaccgcagccagccggtgcctggcgcccctgccccccgcccctctccaaacaccggcagaaaacggagagtgcttgggtggtgggtgctggaggattttccagttctgacacacgtatttatatttggaaagagaccagcaccgagctcggcacctccccggcctctctcttcccagctgcagatgccacacctgctccttcttgctttccccgggggaggaagggggttgtggtcggggagctggggtacaggtttggggagggggaagagaaatttttatttttgaacccctgtgtcccttttgcataagattaaaggaaggaaaagt

SEQ ID NO: 4 - protein, 328 aa, Homo sapiens
MLPRVGCPALPLPPPPLLPLLPLLLLLLGASGGGGGARAEVLFRCPPCTPERLAACGPPPVAPPAAVAAVAGGARMPCAELVREPGCGCCSVCARLEGEACGVYTPRCGQGLRCYPHPGSELPLQALVMGEGTCEKRRDAEYGASPEQVADNGDDHSEGGLVENHVDSTMNMLGGGGSAGRKPLKSGMKELAVFREKVTEQHRQMGKGGKHHLGLEEPKKLRPPPARTPCQQELDQVLERISTMRLPDERGPLEHLYSLHIPNCDKHGLYNLKQCKMSLNGQRGECWCVNPNTGKLIQGAPTIRGEPECHLFYNEQQEACGVHTQRMQ

SEQ ID NO: 5 - DNA, 1433 bp, Homo sapiens
attcggggcgagggaggaggaagaagcggaggaggcggctcccgctcgcagggccgtgcacctgcccgcccgcccgctcgctcgctcgcccgccgcgccgcgctgccgaccgccagcatgctgccgagagtgggctgccccgcgctgccgctgccgccgccgccgctgctgccgctgctgccgctgctgctgctgctactgggcgcgagtggcggcggcggcggggcgcgcgcggaggtgctgttccgctgcccgccctgcacacccgagcgcctggccgcctgcgggcccccgccggttgcgccgcccgccgcggtggccgcagtggccggaggcgcccgcatgccatgcgcggagctcgtccgggagccgggctgcggctgctgctcggtgtgcgcccggctggagggcgaggcgtgcggcgtctacaccccgcgctgcggccaggggctgcgctgctatccccacccgggctccgagctgcccctgcaggcgctggtcatgggcgagggcacttgtgagaagcgccgggacgccgagtatggcgccagcccggagcaggttgcagacaatggcgatgaccactcagaaggaggcctggtggagaaccacgtggacagcaccatgaacatgttgggcgggggaggcagtgctggccggaagcccctcaagtcgggtatgaaggagctggccgtgttccgggagaaggtcactgagcagcaccggcagatgggcaagggtggcaagcatcaccttggcctggaggagccccagcagctgaacccaccccctgccaggactccctgccaacaggaactggaccaggtcctggagcggatctccaccatgcgccttccggatgagcggggccctctggagcacctctactccctgcacatccccaactgtgacaagcatggcctgtacaacctcaaacagtgcaagatgtctctgaacgggcagcgtggggagtgctggtgtgtgaaccccaacaccgggaagctgatccagggagcccccaccatccggggggaccccgagtgtcatctcttctacaatgagcagcaggaggcttgcggggtgcacacccagcggatgcagtagaccgcagccagccggtgcctggcgcccctgccccccgcccctctccaaacaccggcagaaaacggagagtgcttgggtggtgggtgctggaggattttccagttctgacacacgtatttatatttggaaagagaccagcaccgagctcggcacctccccggcctctctcttcccagctgcagatgccacacctgctccttcttgctttccccgggggaggaagggggttgtggtcggggagctggggtacaggtttggggagggggaagagaaatttttatttttgaacccctgtgtcccttttgcataagattaaaggaaggaaaagt

SEQ ID NO: 6 - protein, 328 aa, Homo sapiens
MLPRVGCPALPLPPPPLLPLLPLLLLLLGASGGGGGARAEVLFRCPPCTPERLAACGPPPVAPPAAVAAVAGGARMPCAELVREPGCGCCSVCARLEGEACGVYTPRCGQGLRCYPHPGSELPLQALVMGEGTCEKRRDAEYGASPEQVADNGDDHSEGGLVENHVDSTMNMLGGGGSAGRKPLKSGMKELAVFREKVTEQHRQMGKGGKHHLGLEEPQQLNPPPARTPCQQELDQVLERISTMRLPDERGPLEHLYSLHIPNCDKHGLYNLKQCKMSLNGQRGECWCVNPNTGKLIQGAPTIRGDPECHLFYNEQQEACGVHTQRMQ